(19) United States
(12) Patent Application Publication
(10) Pub. No.: US 2025/0177414 A1
(43) Pub. Date: Jun. 5, 2025

(54) COMBINATION THERAPIES FOR TREATMENT OF BREAST CANCER

(71) Applicants: Genentech, Inc., South San Francisco, CA (US); Hoffmann-La Roche Inc., Little Falls, NJ (US); Pfizer Inc., New York, NY (US)

(72) Inventors: Eirini Thanopoulou, St Albans (GB); Yanling Jin, Hamilton (CA); Guiyuan Lei, Welwyn Garden City (GB); Colin James Neate, Toronto (CA); Noopur Shankar, Belmont, CA (US); Chunyan Song, Redwood City, CA (US); Thomas James Stout, Mill Valley, CA (US)

(73) Assignees: Genentech, Inc., South San Francisco, CA (US); Hoffmann-La Roche Inc., Little Falls, NJ (US); Pfizer Inc., New York, NY (US)

(21) Appl. No.: 18/967,059

(22) Filed: Dec. 3, 2024

Related U.S. Application Data
(60) Provisional application No. 63/713,780, filed on Oct. 30, 2024.
(60) Provisional application No. 63/705,007, filed on Oct. 8, 2024.
(60) Provisional application No. 63/653,783, filed on May 30, 2024.
(60) Provisional application No. 63/607,531, filed on Dec. 7, 2023.
(60) Provisional application No. 63/606,014, filed on Dec. 4, 2023.

(51) Int. Cl. A61K 31/553; A61K 31/519; A61K 31/566; A61P 35/00

(52) U.S. Cl. CPC A61K 31/553; A61K 31/519; A61K 31/566; A61P 35/00

(57) ABSTRACT

Provided are a combination therapy comprising inavolisib, palbociclib and fulvestrant and methods of treating PIC3CA-mutant, hormone receptor positive (HR+) and HER2 negative (HER2−) locally advanced or metastatic breast cancer comprising administering a therapeutically effective amount of inavolisib, palbociclib and fulvestrant. The combination therapy produces a statistically significant and clinically meaningful improvement to the patient as compared to administering palbociclib and fulvestrant alone to a comparable patient.

### CROSS REFERENCE TO RELATED APPLICATIONS

This application claims the benefit of U.S. Provisional Patent Application No. 63/606,014, filed Dec. 4, 2023; U.S. Provisional Patent Application No. 63/607,531, filed Dec. 7, 2023; U.S. Provisional Patent Application No. 63/653,783, filed May 30, 2024; U.S. Provisional Patent Application No. 63/705,007, filed Oct. 8, 2024; and U.S. Provisional Patent Application No. 63/713,780, filed Oct. 30, 2024; the contents of which are incorporated by reference herein in their entireties.

### FIELD OF THE INVENTION

The invention relates to a combination of inavolisib, palbociclib and fulvestrant for the treatment of advanced hormone receptor positive (HR+), HER2 Negative (HER2−) breast cancer with a PIK3CA mutation.

### BACKGROUND

Globally, breast cancer is the second most common invasive malignancy and the most common cause of cancer-related mortality in women, with a 5-year survival rate following metastatic diagnosis of approximately 15%.

Phosphatidylinositol 3-kinase (PI3K) is a lipid kinase that upon activation by growth factor receptors and integrins regulates cell proliferation, survival, and migration. PI3K catalyzes the phosphorylation of phosphatidylinositol-4,5-bisphosphate (PIP₂) to generate phosphatidylinositol-3,4,5-triphosphate (PIP₃), a second messenger involved in the phosphorylation of AKT and other components in the AKT/mTOR pathway. Up to 70% of breast cancers have some form of molecular aberration of the PI3K/AKT/mTOR pathway. Activating mutations in PIK3CA, encoding the p110α subunit of PI3K, are highly prevalent in breast cancer and solid tumor malignancies.

Hormone receptor (HR)-positive breast cancer is the most prevalent type of all breast cancers. A defining feature of HR-positive breast cancer is that its tumour cells have receptors that attach to one or both hormones—estrogen or progesterone—which can contribute to tumour growth. People diagnosed with HR-positive metastatic breast cancer often face the risk of disease progression and treatment side effects, creating a need for additional treatment options. The PI3K signalling pathway is commonly dysregulated in HR-positive breast cancer, often due to activating PIK3CA mutations, which have been identified as a potential mechanism for resistance to endocrine therapy and CDK4/6 inhibitors.

### SUMMARY OF THE INVENTION

The present disclosure provides a combination therapy comprising inavolisib, palbociclib and fulvestrant for the treatment of breast cancers, e.g., a PIK3CA mutated, hormone receptor positive and HER2 negative locally advanced or metastatic breast cancer.

In one aspect, the combination therapy comprising inavolisib, palbociclib and fulvestrant described herein produces a statistically significant and clinically meaningful improvement to patients having breast cancers (e.g., a PIK3CA mutant/mutated, hormone receptor positive and HER2 negative locally advanced or metastatic breast cancer) compared to administering palbociclib and fulvestrant alone (not including inavolisib) to a comparable patient.

The disclosure further provides methods of treating Hormone Receptor Positive and HER2 Negative (HR+/HER2−) locally advanced or metastatic breast cancer in a patient comprising administering a therapeutically effective amount of inavolisib, palbociclib and fulvestrant.

In some embodiments, the patient has hormone receptor-positive, Her2-negative, locally advanced or metastatic breast cancer with a PIK3CA mutation.

Also provided is a combination for use in treating Hormone Receptor Positive and HER2 Negative (HR+/HER2−) locally advanced or metastatic breast cancer wherein said combination comprises inavolisib, palbociclib and fulvestrant.

In some embodiments, the hormone receptor-positive, Her2-negative, locally advanced or metastatic breast cancer is PIK3CA mutant/mutated or has one or more PIK3CA mutations.

In some embodiments, inavolisib is adminstered at a 9 mg daily dose.

In some embodiments, provided is a method of treating hormone receptor positive and HER2 negative locally advanced or metastatic breast cancer in a patient having PIK3CA mutant, hormone receptor positive and HER2 negative locally advanced or metastatic breast cancer, the method comprising administering to the patient a combination therapy comprising inavolisib, palbociclib and fulvestrant, wherein said combination therapy is administered over a 28-day cycle.

In some embodiments, provided is a method of treating hormone receptor positive and HER2 negative locally advanced or metastatic breast cancer in a patient having PIK3CA mutated, hormone receptor positive and HER2 negative locally advanced or metastatic breast cancer, the method comprising administering to the patient a combination therapy comprising a dosing regimen comprising:
a. administering inavolisib QD on days 1-28 of a first 28-day cycle;
b. administering palbociclib QD on days 1-21 of a first 28-day cycle; and
c. administering fulvestrant on days 1 and 15 of a first 28-day cycle,
   wherein the combination therapy produces a statistically significant and clinically meaningful improvement to the patient as compared to administering palbociclib and fulvestrant alone to a comparable patient.

In some of these embodiments, the method further comprises one or more additional 28-day cycles comprising:
a. administering inavolisib on days 1-28 of each additional 28-day cycle;
b. administering palbociclib on days 1-21 of each additional 28-day cycle; and
c. administering fulvestrant on day 1 of each additional 28-day cycle (or approximately once every 4 weeks).

Also provided is a combination for use in treating a PIK3CA mutant, hormone receptor positive and HER2 negative locally advanced or metastatic breast cancer, wherein said combination comprises inavolisib, palbociclib and fulvestrant, wherein said combination is administered over a 28-day cycle, and wherein said combination comprising inavolisib, palbociclib and fulvestrant produces a statistically significant and clinically meaningful improvement to the patient compared to palbociclib and fulvestrant alone (not including inavolisib) administered to a comparable patient.

Also provided is a combination for use in treating a PIK3CA mutant, hormone receptor positive and HER2 negative locally advanced or metastatic breast cancer, wherein said combination is administered in a combination therapy comprising a dosing regimen comprising:
a. administering inavolisib QD on days 1-28 of a first 28-day cycle;
b. administering palbociclib QD on days 1-21 of a first 28-day cycle; and
c. administering fulvestrant on days 1 and 15 of a first 28-day cycle,
   and wherein the combination comprising inavolisib, palbociclib and fulvestrant produces a statistically significant and clinically meaningful improvement to the patient compared to palbociclib and fulvestrant alone (not including inavolisib) administered to a comparable patient.

Also provided is a use of inavolisib in the manufacture of a medicament for treating a PIK3CA mutated, hormone receptor positive and HER2 negative locally advanced or metastatic breast cancer, wherein the treating comprises administration of the medicament in combination with compositions comprising palbociclib and fulvestrant, wherein administration of the medicament in combination with compositions comprising palbociclib and fulvestrant produces a statistically significant and clinically meaningful improvement to the patient as compared to administration of palbociclib and fulvestrant alone. In some embodiments, the treating comprises administration of the medicament in combination with a composition comprising palbociclib, or a pharmaceutically acceptable salt thereof, and a composition comprising fulvestrant, or a pharmaceutically acceptable salt thereof.

Also provided is a use of a combination in the manufacture of a medicament for treating a PIK3CA mutated, hormone receptor positive and HER2 negative locally advanced or metastatic breast cancer, wherein said combination comprises inavolisib, palbociclib and fulvestrant, wherein said combination is administered over a 28-day cycle, and wherein administration of the combination comprising inavolisib, palbociclib and fulvestrant produces a statistically significant and clinically meaningful improvement to the patient compared to administration of palbociclib and fulvestrant alone (not including inavolisib) to a comparable patient.

Also provided is a use of a combination in the manufacture of a medicament for treating a PIK3CA mutant, hormone receptor positive and HER2 negative locally advanced or metastatic breast cancer, wherein said combination is administered in a combination therapy comprising a dosing regimen comprising:
a. administering inavolisib QD on days 1-28 of a first 28-day cycle;
b. administering palbociclib QD on days 1-21 of a first 28-day cycle; and
c. administering fulvestrant on days 1 and 15 of a first 28-day cycle,
   and wherein administration of the combination comprising inavolisib, palbociclib and fulvestrant produces a statistically significant and clinically meaningful improvement to the patient compared to administration of palbociclib and fulvestrant alone (not including inavolisib) to a comparable patient.

In some of these embodiments, the dosing regimen further comprises one or more additional 28-day cycles comprising:
a. administering inavolisib on days 1-28 of each additional 28-day cycle;
b. administering palbociclib on days 1-21 of each additional 28-day cycle; and
c. administering fulvestrant on day 1 of each additional 28-day cycle (or approximately once every 4 weeks).

In some of these embodiments, inavolisib is administered at an amount of 3, 6 or 9 mg, e.g., in one or more oral tablets. In some of these embodiments, inavolisib is administered at an amount of 9 mg, e.g., in an oral tablet. In some embodiments, palbociclib is administered at an amount of 125 mg, e.g., in an oral capsule or tablet. In some embodiments, fulvestrant is administered at an amount of 500 mg, e.g., by intramuscular (IM) injection or infusion.

In a further aspect, provided is a method of inhibiting tumor growth or producing/increasing tumor regression in a patient having PIK3CA mutated, hormone receptor positive and HER2 negative locally advanced or metastatic breast cancer, the method comprising administering to the patient a combination therapy according to the methods detailed herein.

In another aspect, provided is a combination for use in inhibiting tumor growth or producing/increasing tumor regression in a patient having PIK3CA mutated, hormone receptor positive and HER2 negative locally advanced or metastatic breast cancer, according to the combination for use detailed herein.

In another aspect, provided is a use of a combination in the manufacture of a medicament for inhibiting tumor growth or producing/increasing tumor regression in a patient having PIK3CA mutant, hormone receptor positive and HER2 negative locally advanced or metastatic breast cancer, according to the combinations or uses detailed herein.

In some of these embodiments, the patient has locally advanced or metastatic breast cancer not amenable to curative therapy. In some embodiments, the patient has locally advanced breast cancer. In some embodiments, the patient has metastatic breast cancer. In some embodiments, the patient has PIK3CA-mutated, ER-positive, PR-positive and HER2-negative locally advanced or metastatic breast cancer. In some embodiments, the patient has no liver metastasis. In some embodiments, the breast cancer is endocrine resistant. In some embodiments, the patient has progression of disease during adjuvant endocrine treatment or within 12 months of completing adjuvant endocrine therapy (e.g., with an aromatase inhibitor or tamoxifen). In some embodiments, the patient has progression of disease during the first two years of adjuvant endocrine treatment. In one embodiment, the adjuvant endocrine therapy is tamoxifen only. In some embodiments, the patient has progression of disease during adjuvant endocrine treatment or within 12 months of completing adjuvant endocrine therapy with tamoxifen. In some embodiments, the patient has progression of disease during the first two years of adjuvant endocrine therapy. In some embodiments, the patient has progression of disease during the first two years of endocrine therapy with tamoxifen. In some embodiments, he patient has adequate hematologic and organ function within 14 days prior to initiation of study treatment. In some embodiments, the patient is post-menopausal (e.g., a post-menopausal women). In some embodiments, the patient is not post-menopausal. In some embodiments, the patient is premenopausal or perimenopausal (e.g., a premenopausal or a perimenopausal women). In some embodiments, the patient is male. In some embodiments, the patient is an adult. In some embodiments, the patient is <65 years of age. In some embodiments, the patient is an adult younger than 65 years of age. In some embodiments, the patient is an Asian. In some embodiments, the breast cancer is as detected by an FDA-approved test.

Further provided is a method of preventing or delaying development of resistance of a tumor (e.g., breast cancer) to a therapy containing palbociclib, comprising administering a combination therapy comprising inavolisib, palbociclib and fulvestrant. In some embodiments, the combination therapy is administered according to any methods as detailed herein.

Also provided is a combination for use in preventing or delaying development of resistance of a tumor (e.g., breast cancer) to a therapy containing palbociclib, wherein said combination comprises inavolisib, palbociclib and fulvestrant. In some embodiments, said combination is administered according to any uses as detailed herein.

Also provided is a use of a combination in the manufacture of a medicament for preventing or delaying development of resistance of a tumor (e.g., breast cancer) to a therapy containing palbociclib, wherein said combination comprises inavolisib, palbociclib and fulvestrant. In some embodiments, said combination is administered according to any uses as detailed herein.

Also provided is a combination therapy comprising inavolisib, palbociclib and fulvestrant for the treatment of adults with endocrine-resistant, PIK3CA mutated, hormone receptor (HR)-positive, human epidermal growth factor receptor 2 (HER2)-negative, locally advanced or metastatic breast cancer, as detected by an FDA-approved test, following recurrence on or after completing adjuvant endocrine therapy. Also provided are the following patient sub-populations, which may be taken individually, or combined in any way to generate a different patient sub-population on which a therapy described herein can be used. The sub-populations are: the patient has progression of disease during the first two years of adjuvant endocrine treatment (primary endocrine resistance); the adjuvant endocrine therapy is with tamoxifen only; the patient is not post-menopausal; the patient has PIK3CA mutated, hormone receptor positive and HER2 negative metastatic breast cancer; the patient has PIK3CA mutated, estrogen receptor positive, progesterone receptor positive, and HER2 negative locally advanced or metastatic breast cancer; the patient is an adult; the patient is <65 years of age; and the patient is an Asian adult.

### BRIEF DESCRIPTION OF THE DRAWINGS

FIG. 1 shows the study design of INAVO120.

FIG. 2 shows the Kaplan-Meier Plot of investigator assessed PFS in the patients whose PIK3CA mutation-positive status was identified by the central FMI testing.

FIG. 3 shows the Kaplan-Meier plot the OS trend at an interim analysis in the patients whose PIK3CA mutation-positive status was identified by the central FMI testing.

FIG. 4 shows the Kaplan-Meier plot of the DOR in the patients with response whose PIK3CA mutation-positive status was identified by the central FMI testing.

FIG. 5 shows the Kaplan-Meier plot of the PFS in all patients enrolled in the INAVO120 study.

FIG. 6 shows the Kaplan-Meier plot of the time from randomization to end or discontinuation of next-line treatment, or death from any cause (proxy for PFS2).

FIG. 7 shows the Kaplan-Meier plot of the time from randomization to first subsequent chemotherapy after treatment discontinuation.

FIG. 8 shows a graphical representation of the key selected AEs.

FIG. 9 shows the Kaplan-Meier plot of the time to confirmed clinical meaningful deterioration in worst pain severity.

FIG. 10 shows a graphical representation of the percent of patients based on the baseline and worst post-baseline PRO-CTCAE scores.

FIG. 11 shows the “bother” the patients experienced during the course of the treatment.

FIG. 12 shows the Kaplan-Meier curve for Investigator-Assessed Progression-Free Survival in INAVO120.

FIG. 13 shows the investigator-assessed progression-free survival for the full analysis set.

FIG. 14 shows the key subgroups. Sample size is relatively small for many groups; therefore, the analysis is unstratified including for ‘all patients’; hence, the difference in the hazard ratio is relative to that for the stratified full analysis set analysis.

FIG. 15 shows the Kaplan-Meier Curve for the overall survival rate.

FIG. 16 shows the overall response rate for the full analysis set.

FIG. 17 shows the duration of response for the full analysis set.

### DETAILED DESCRIPTION OF THE INVENTION

### Definitions

The words “comprise,” “comprising,” “include,” “including,” and “includes” when used in this specification and claims are intended to specify the presence of stated features, integers, components, or steps, but they do not preclude the presence or addition of one or more other features, integers, components, steps, or groups thereof.

The terms “treat” and “treatment” refer to both therapeutic treatment and prophylactic or preventative measures, wherein the object is to prevent or slow down (lessen) an undesired physiological change or disorder, such as the growth, development or spread of cancer. For purposes of this invention, beneficial or desired clinical results include, but are not limited to, alleviation of symptoms, diminishment of extent of disease, stabilized (i.e., not worsening) state of disease, delay or slowing of disease progression, amelioration or palliation of the disease state, and remission (whether partial or total), whether detectable or undetectable. “Treatment” can also mean prolonging survival as compared to expected survival if not receiving treatment. Those in need of treatment include those already with the condition or disorder as well as those prone to have the condition or disorder or those in which the condition or disorder is to be prevented.

The phrase “therapeutically effective amount” means an amount of a compound of the present invention that (i) treats the particular disease, condition, or disorder, (ii) attenuates, ameliorates, or eliminates one or more symptoms of the particular disease, condition, or disorder, or (iii) prevents or delays the onset of one or more symptoms of the particular disease, condition, or disorder described herein. In the case of cancer, the therapeutically effective amount of the drug may reduce the number of cancer cells; reduce the tumor size; inhibit (i.e., slow to some extent and preferably stop) cancer cell infiltration into peripheral organs; inhibit (i.e., slow to some extent and preferably stop) tumor metastasis; inhibit, to some extent, tumor growth; and/or relieve to some extent one or more of the symptoms associated with the cancer. To the extent the drug may prevent growth and/or kill existing cancer cells, it may be cytostatic and/or cytotoxic. For cancer therapy, efficacy can be measured, for example, by assessing the time to disease progression (TTP) and/or determining the response rate (RR).

“Time to progression” or “TTP” refers to the time from randomization until objective tumor progression.

“Objective response rate” or “ORR” refers to the proportion of patients with a confirmed complete response or partial response on two consecutive occasions≥4 weeks apart, as determined by the investigator according to RECIST v1.1

“Best overall response rate” or “BOR” refers to the proportion of patients with a CR or PR, as determined by the investigator according to RECIST v1.1

“Duration of response” or “DOR” refers to the time from the first occurrence of a documented objective response to disease progression, as determined by the investigator according to RECIST v1.1, or death from any cause, whichever occurs first.

“Clinical benefit rate” or “CBR” refers to the proportion of patients with stable disease for at least 24 weeks or with confirmed complete or partial response, as determined by the investigator according to RECIST v1.1.

“Overall survival” or “OS” refers to the time from enrollment to death from any cause.

“Time to deterioration (TTD) in pain” refers to the time from randomization to the first documentation of a ≥2-point increase from baseline on the “worst pain” item from the Brief Pain Inventory-Short Form (BPI-SF).

“Time to deterioration (TTD) in Physical Function' refers to the time from randomization to the first documentation of a ≥10-point decrease from baseline in the European Organisation for Research and Treatment of Cancer Quality of Life-Core 30 Questionnaire (EORTC QLQ-C30) Physical Function scale (items 1-5).

“Time to deterioration (TTD) in Role Function” refers to the time from randomization to the first documentation of a 10-point decrease from baseline in the EORTC QLQ-C30 Role Function scale (items 6 and 7).

“Time to deterioration (TTD) in global health status (GHS)/health-related quality of life (HRQoL)” refers to the time from randomization to the first documentation of a 10-point decrease from baseline in the EORTC QLQ-30 GHS/HRQoL scale (items 29 and 30).

“Progression free survival” or “PFS” refers to the time from enrollment to the date of the first recorded occurrence of disease progression, as determined by the investigator using RECIST v1.1 or death from any cause, whichever occurs first.

“Complete response” or “CR” refers to the disappearance of all target lesions and non-target lesions and (if applicable) normalization of tumor marker level.

“Partial response”, “PR” or “Non-CR/Non-PrD” refers to persistence of one or more non-target lesions and/or (if applicable) maintenance of tumor marker level above the normal limits. A PR can also refer to 30% decrease in sum of diameters of target lesions, in the absence of CR, new lesions, and unequivocal progression in non-target lesions.

“Progressive disease” or “PrD” refers to 20% increase in sum of diameters of target lesions, unequivocal progression in non-target lesions, and/or appearance of new lesions.

“Stable disease” or “SD” refers to neither sufficient shrinkage to qualify for CR or PR nor sufficient increase growth of tumor to qualify for PrD.

An “administration period” or “cycle” refers to a period of time comprising administration of one or more agents described herein and an optional period of time comprising no administration of one or more of the agents described herein. For example, a cycle can be 28 days in total length and include administration of one or more agents for 21 days and a rest period of 7 days. A “rest period” refers to a period of time where at least one of the agents described herein are not administered. In one embodiment, a rest period refers to a period of time where none of the agents described herein are administered.

A “dosing regimen” refers to a period of administration of the agents described herein comprising one or more cycles, where each cycle can include administration of the agents described herein at different times or in different amounts.

“QD” refers to administration of a compound once daily.

A graded adverse event refers to the severity grading scale as established for by NCI CTCAE. In one embodiment, the adverse event is graded in accordance with the table below.

| Grade | Severity |
|---|---|
| 1 | Mild; asymptomatic or mild symptoms; clinical or diagnostic |
| | observations only; or intervention not indicated |
| 2 | Moderate; minimal, local, or non-invasive intervention |
| | indicated; or limiting age-appropriate instrumental |
| | activities of daily living |
| 3 | Severe or medically significant, but not immediately |
| | life-threatening; hospitalization or prolongation of |
| | hospitalization indicated; disabling; or limiting |
| | self-care activities of daily living |
| 4 | Life-threatening consequences or urgent intervention indicated |
| 5 | Death related to adverse event |

The term “detection” includes any means of detecting, including direct and indirect detection.

The term “prognosis” is used herein to refer to the prediction of the likelihood of cancer-attributable death or progression, including, for example, recurrence, metastatic spread, and drug resistance, of a neoplastic disease, such as cancer.

The term “prediction” (and variations such as predicting) is used herein to refer to the likelihood that a patient will respond either favorably or unfavorably to a drug or set of drugs. In one embodiment, the prediction relates to the extent of those responses. In another embodiment, the prediction relates to whether and/or the probability that a patient will survive following treatment, for example treatment with a particular therapeutic agent and/or surgical removal of the primary tumor, and/or chemotherapy for a certain period of time without cancer recurrence. The predictive methods of the invention can be used clinically to make treatment decisions by choosing the most appropriate treatment modalities for any particular patient. The predictive methods of the present invention are valuable tools in predicting if a patient is likely to respond favorably to a treatment regimen, such as a given therapeutic regimen, including for example, administration of a given therapeutic agent or combination, surgical intervention, chemotherapy, etc., or whether long-term survival of the patient, following a therapeutic regimen is likely.

The term “increased resistance” to a particular therapeutic agent or treatment option, when used in accordance with the invention, means decreased response to a standard dose of the drug or to a standard treatment protocol.

“Response” can be assessed using any endpoint indicating a benefit to the patient, including, without limitation, (1) inhibition, to some extent, of tumor growth, including slowing down or complete growth arrest; (2) reduction in the number of tumor cells; (3) reduction in tumor size; (4) inhibition (e.g., reduction, slowing down or complete stopping) of tumor cell infiltration into adjacent peripheral organs and/or tissues; (5) inhibition (e.g., reduction, slowing down or complete stopping) of metastasis; (6) enhancement of anti-tumor immune response, which may, but does not have to, result in the regression or rejection of the tumor; (7) relief, to some extent, of one or more symptoms associated with the tumor; (8) increase in the length of survival following treatment; and/or (9) decreased mortality at a given point of time following treatment.

A “biomarker” is a characteristic that is objectively measured and evaluated as an indicator of normal biological processes, pathogenic processes, or pharmacological responses to a therapeutic intervention. Biomarkers may be of several types: predictive, prognostic, or pharmacodynamics (PD). Predictive biomarkers predict which patients are likely to respond or benefit from a particular therapy. Prognostic biomarkers predict the likely course of the patient's disease and may guide treatment. Pharmacodynamic biomarkers confirm drug activity, and enables optimization of dose and administration schedule.

“Change” or “modulation” of the status of a biomarker, including a PIK3CA mutation or set of PIK3CA mutations, as it occurs in vitro or in vivo is detected by analysis of a biological sample using one or more methods commonly employed in establishing pharmacodynamics (PD), including: (1) sequencing the genomic DNA or reverse-transcribed PCR products of the biological sample, whereby one or more mutations are detected; (2) evaluating gene expression levels by quantitation of message level or assessment of copy number; and (3) analysis of proteins by immunohistochemistry (IHC), immunocytochemistry, ELISA, or mass spectrometry whereby degradation, stabilization, or post-translational modifications of the proteins such as phosphorylation or ubiquitination is detected.

A “chemotherapeutic agent” is a biological (large molecule) or chemical (small molecule) compound useful in the treatment of cancer, regardless of mechanism of action.

The term “package insert” is used to refer to instructions customarily included in commercial packages of therapeutic products, that contain information about the indications, usage, dosage, administration, contraindications and/or warnings concerning the use of such therapeutic products.

The phrase “pharmaceutically acceptable salt” as used herein, refers to pharmaceutically acceptable organic or inorganic salts of a compound of the invention. Exemplary salts include, but are not limited, to sulfate, citrate, acetate, oxalate, chloride, bromide, iodide, nitrate, bisulfate, phosphate, acid phosphate, isonicotinate, lactate, salicylate, acid citrate, tartrate, oleate, tannate, pantothenate, bitartrate, ascorbate, succinate, maleate, gentisinate, fumarate, gluconate, glucuronate, saccharate, formate, benzoate, glutamate, methanesulfonate “mesylate”, ethanesulfonate, benzenesulfonate, p-toluenesulfonate, and pamoate (i.e., 1,1′-methylene-bis-(2-hydroxy-3-naphthoate)) salts. A pharmaceutically acceptable salt may involve the inclusion of another molecule such as an acetate ion, a succinate ion or other counter ion. The counter ion may be any organic or inorganic moiety that stabilizes the charge on the parent compound. Furthermore, a pharmaceutically acceptable salt may have more than one charged atom in its structure. Instances where multiple charged atoms are part of the pharmaceutically acceptable salt can have multiple counter ions. Hence, a pharmaceutically acceptable salt can have one or more charged atoms and/or one or more counter ion.

The desired pharmaceutically acceptable salt may be prepared by any suitable method available in the art. For example, treatment of the free base with an inorganic acid, such as hydrochloric acid, hydrobromic acid, sulfuric acid, nitric acid, methanesulfonic acid, phosphoric acid and the like, or with an organic acid, such as acetic acid, maleic acid, succinic acid, mandelic acid, fumaric acid, malonic acid, pyruvic acid, oxalic acid, glycolic acid, salicylic acid, a pyranosidyl acid, such as glucuronic acid or galacturonic acid, an alpha hydroxy acid, such as citric acid or tartaric acid, an amino acid, such as aspartic acid or glutamic acid, an aromatic acid, such as benzoic acid or cinnamic acid, a sulfonic acid, such as p-toluenesulfonic acid or ethanesulfonic acid, or the like. Acids which are generally considered suitable for the formation of pharmaceutically useful or acceptable salts from basic pharmaceutical compounds are discussed, for example, by P. Stahl et al, Camille G. (eds.) Handbook of Pharmaceutical Salts. Properties, Selection and Use. (2002) Zurich: Wiley-VCH; S. Berge et al, Journal of Pharmaceutical Sciences (1977) 66(1) 1 19; P. Gould, International J. of Pharmaceutics (1986) 33 201 217; Anderson et al, The Practice of Medicinal Chemistry (1996), Academic Press, New York; Remington's Pharmaceutical Sciences, 18^{th} ed., (1995) Mack Publishing Co., Easton PA; and in The Orange Book (Food & Drug Administration, Washington, D.C. on their website). These disclosures are incorporated herein by reference thereto.

The phrase “pharmaceutically acceptable” indicates that the substance or composition must be compatible chemically and/or toxicologically, with the other ingredients comprising a formulation, and/or the patient being treated therewith.

The term “synergistic” as used herein refers to a therapeutic combination which is more effective than the additive effects of the two or more single agents. A determination of a synergistic interaction between a compound of inavolisib or a pharmaceutically acceptable salt thereof, and one or more chemotherapeutic agent may be based on the results obtained from the assays described herein. The results of these assays can be analyzed using the Chou and Talalay combination method and Dose-Effect Analysis with CalcuSyn® software in order to obtain a Combination Index (Chou and Talalay, 1984, Adv. Enzyme Regul. 22:27-55). The combinations provided by this invention have been evaluated in several assay systems, and the data can be analyzed utilizing a standard program for quantifying synergism, additivism, and antagonism among anticancer agents described by Chou and Talalay, in “New Avenues in Developmental Cancer Chemotherapy,” Academic Press, 1987, Chapter 2. Combination Index values less than 0.8 indicates synergy, values greater than 1.2 indicate antagonism and values between 0.8 and 1.2 indicate additive effects. The combination therapy may provide “synergy” and prove “synergistic”, i.e., the effect achieved when the active ingredients used together is greater than the sum of the effects that results from using the compounds separately. A synergistic effect may be attained when the active ingredients are: (1) co-formulated and administered or delivered simultaneously in a combined, unit dosage formulation; (2) delivered by alternation or in parallel as separate formulations; or (3) by some other regimen. When delivered in alternation therapy, a synergistic effect may be attained when the compounds are administered or delivered sequentially, e.g., by different injections in separate syringes or in separate pills or tablets. In general, during alternation therapy, an effective dosage of each active ingredient is administered sequentially, i.e., serially, whereas in combination therapy, effective dosages of two or more active ingredients are administered together. Combination effects were evaluated using both the BLISS independence model and the highest single agent (HSA) model (Lehár et al. 2007, Molecular Systems Biology 3:80). BLISS scores quantify degree of potentiation from single agents and a BLISS score>0 suggests greater than simple additivity. An HSA score>0 suggests a combination effect greater than the maximum of the single agent responses at corresponding concentrations.

### Clinical Compounds

### Inavolisib:

Inavolisib is an oral therapy with high in vitro potency and selectivity for PI3Kα inhibition and the ability to specifically trigger the breakdown of mutant PI3Kα protein. Inavolisib has been designed to help minimise the overall toxicity of treatment and is differentiated from other PI3K inhibitors due to its high in vitro potency and specificity for the PI3K alpha (PI3Kα) isoform inhibition, together with its unique mechanism of action, that leads to specific degradation of mutant PI3K alpha. See Juric et al. A phase I/Ib study of inavolisib (GDC-0077) in combination with fulvestrant in patients (pts) with PIK3CA-mutated hormone receptor-positive/HER2-negative (HR+/HER2−) metastatic breast cancer. Presented at San Antonio Breast Cancer Symposium, 2020 Dec. 7-10; San Antonio, USA. Abstract #P5-17-05; and Hong R., et al. GDC-0077 is a selective PI3K alpha inhibitor that demonstrates robust efficacy in PIK3CA mutant breast cancer models as a single agent and in combination with standard of care therapies. Cancer Res. 2018; 78(4):4-1. With this unique dual mechanism of action, inavolisib provides well-tolerated, durable disease control and potentially improved outcomes for people with HR-positive/HER2-negative, PIK3CA-mutated advanced breast cancer. PIK3CA mutations can lead to mutated PI3Kα protein which contributes to uncontrolled tumour growth, disease progression and resistance to endocrine-based treatment.

Inavolisib, CAS Registry Number 2060571-02-8, Genentech, Inc., U.S. Pat. No. 9,650,393; named as (S)-2-((2-((S)-4-(difluoromethyl)-2-oxooxazolidin-3-yl)-5,6-dihydrobenzo[f]imidazo[1,2-d][1,4]oxazepin-9-yl)amino)propanamide, has the structure:

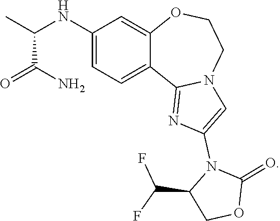 [SMILES: C[C@H](Nc1ccc2c(c1)OCCn1cc(N3C(=O)OC[C@H]3C(F)F)nc1-2)C(N)=O]

Inavolisib is also known as inavolisib, RG6114, RO7113755, or chemical name (2S)-2-[[2-[(4S)-4-(Difluoromethyl)-2-oxo-3-oxazolidinyl]-5,6-dihydroimidazo[1,2-d][1,4]benzoxazepin-9-yl]amino]propanamide.

Inavolisib exerts its activity by binding to the ATP binding site of PI3K, thereby inhibiting the phosphorylation of membrane-bound 4,5-phosphatidylinositol bisphosphate (PIP₂) to 3,4,5-phosphatidylinositol triphosphate (PIP₃). Inhibiting the phosphorylation of PIP₂ to PIP₃ decreases downstream activation of AKT and pS6, resulting in decreased cellular proliferation, metabolism, and angiogenesis. Nonclinical studies demonstrate that inavolisib specifically degrades mutant p110 alpha, inhibits proliferation and induces apoptosis of PIK3CA-mutant breast cancer cell lines, inhibits tumor growth in human breast xenograft models harboring PIK3CA mutations, and reduces downstream PI3K-pathway markers, including pAKT (phosphorylated form of AKT), pPRAS40, and pS6.

### Palbociclib:

Palbociclib is a selective inhibitor of the cyclin-dependent kinases CDK4 and CDK6 (Finn et al (2009) Breast cancer research: BCR 11 (5):R77; Rocca et al (2014) Expert Opin Pharmacother 15 (3):407-20; U.S. Pat. Nos. 6,936,612; 7,863,278; 7,208,489; 7,456,168; U.S. RE47739; U.S. Ser. No. 10/723,730). Palbociclib can be prepared and characterized as described in U.S. Pat. No. 7,345,171. IBRANCE® is approved for the treatment of breast cancer.

Palbociclib (PD-0332991, IBRANCE®, Pfizer, Inc., CAS Reg. No. 571190-30-2), named as 6-acetyl-8-cyclopentyl-5-methyl-2-(5-(piperazin-1-yl)pyridin-2-ylamino)pyrido[2,3-d]pyrimidin-7(8H)-one, has the structure:

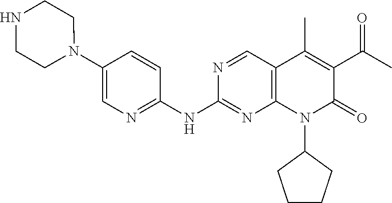 [SMILES: CC(=O)c1c(C)c2cnc(Nc3ccc(N4CCNCC4)cn3)nc2n(C2CCCC2)c1=O]

Palbociclib is a CDK4/6 inhibitor and, in combination with letrozole or fulvestrant, an effective treatment for postmenopausal patients with HR+(positive)/HER2− (negative) breast cancer. In combination with letrozole or fulvestrant, the main toxicity of palbociclib is neutropenia (Finn et al (2015) Lancet Oncol 16:25-35; Turner et al (2015) N Engl J Med 373:209-19). In combination with letrozole, 36% of patients required ≥1 dose reduction of palbociclib; dose holds and cycle delays were reported in 70% and 68% of patients, respectively (Finn et al (2016) J Clin Oncol 34(suppl; abstr 507)). In combination with fulvestrant, 34% of patients required ≥1 dose reduction of palbociclib; dose holds and cycle delays were reported in 54% and 36% of patients, respectively (Cristofanilli et al. (2016) Lancet Oncol 17:425-39). Myelosuppression is a potential toxicity of inavolisib.

### Fulvestrant:

Fulvestrant is an ER antagonist and an effective treatment for postmenopausal patients with HR+ breast cancer that is relatively well tolerated. The expected toxicities for inavolisib and fulvestrant are not overlapping.

Fulvestrant (FASLODEX®, AstraZeneca, CAS Reg. No. 129453-61-8) is approved by the FDA for treatment of hormone receptor-positive (HR+) metastatic breast cancer in postmenopausal women with disease progression following anti-estrogen therapy (Kansra (2005) Mol Cell Endocrinol 239(1-2):27-36; Flemming et al (2009) Breast Cancer Res Treat. May; 115(2):255-68; Valachis et al (2010) Crit Rev Oncol Hematol. March; 73(3):220-7). Fulvestrant is an estrogen receptor (ER) antagonist with no agonist effects, which works both by down-regulating and by degrading the estrogen receptor (Croxtall (2011) Drugs 71(3):363-380). Fulvestrant is also a selective estrogen receptor down-regulator (SERD).

Fulvestrant is named as (7α,17β)-7-{9-[(4,4,5,5,5-pentafluoropentyl)sulfinyl]nonyl}estra-1,3,5(10)-triene-3,17-diol and has the structure:

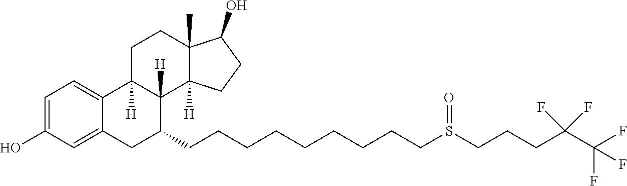

Fulvestrant belongs to a class of reversible steroidal ER antagonists that directly competes with estrogen for ER binding and is devoid of the partial agonist properties of tamoxifen. Upon binding to ER, it blocks estrogen signaling and increases the degradation of ER protein. The affinity of fulvestrant for the ER is approximately 100-fold greater than that of tamoxifen (Howell et al. (2000) Cancer 89:817-25). Fulvestrant (250 mg once monthly) was approved by the FDA in 2002 and by the EMA in 2004 for the treatment of HR-positive MBC in postmenopausal women with disease progression following anti-estrogen therapy. In multicenter Phase III studies, fulvestrant was found to be at least equivalent to anastrozole (a non-steroidal AI) in the second-line setting (Howell et al. (2002) J Clin Oncol 20:3396-3403; Osborne C K, et al (2002) J Clin Oncol 20:3386-95). Fulvestrant is also as active as tamoxifen for the first-line treatment of advanced breast cancer (Howell et al. (2004) J Clin Oncol 22:1605-1613) and displays a level of activity in patients in the post-AI metastatic disease setting similar to that of the non-steroidal AI exemestane (Chia et al. (2008) J Clin Oncol 26:1664-1670). High-dose fulvestrant (500 mg once monthly) has been demonstrated to be at least as effective as anastrozole in terms of clinical benefit rate (CBR) and overall response rate and to be associated with significantly longer time to progression for the first-line treatment of women with advanced HR-positive breast cancer (Robertson et al. (2009) J Clin Oncol 27:4530-4535). High-dose fulvestrant recently demonstrated superior progression-free survival (PFS) in women with ER-positive advanced breast cancer treated with 500 mg versus patients treated with 250 mg (Di Leo et al. (2010) J Clin Oncol 28:4594-4600). Fulvestrant (250 mg and 500 mg) was well tolerated in these studies and produced fewer estrogenic effects than did tamoxifen and resulted in less arthralgia than did the AI anastrozole (Osborne et al. (2002) J Clin Oncol 20:3386-3395). These results led to the approval of 500 mg fulvestrant given once a month as the currently approved recommended dose in the United States and the European Union (in 2010) for postmenopausal women whose disease has spread after treatment with an AI. These studies demonstrate that fulvestrant is an important treatment option for patients with advanced breast cancer and, as such, is considered appropriate control therapy for the present study.

### Combination Therapy

In one aspect, provided is a combination or combination therapy comprising inavolisib, palbociclib and fulvestrant, which produces a statistically significant and clinically meaningful improvement in patients having a PIK3CA mutated, hormone receptor positive and HER2 negative locally advanced or metastatic breast cancer compared to palbociclib and fulvestrant alone (not including inavolisib).

Inavolisib in combination with palbociclib and fulvestrant (as a first-line treatment) more than doubled progression-free survival in patients having a PIK3CA mutated, hormone receptor positive, HER2 negative, endocrine-resistant, locally advanced or metastatic breast cancer, compared to palbociclib and fulvestrant alone in a similar patient. The inavolisib combination has the potential to address resistance to treatment and poor prognosis associated with PIK3CA mutations.

The combinations or combination therapies described herein can be provided as a kit comprising one or more of the agents for administration. In one embodiment, the kit includes inavolisib and fulvestrant. In one embodiment, the kit includes inavolisib, palbociclib and fulvestrant. In one embodiment, the agents of the combination or combination therapy described herein are supplied in a kit in a form ready for administration or, for example, for reconstitution. Kits described herein can include instructions such as package inserts. In one embodiment, the instructions are package inserts—one for each agent in the kit.

Further provided are kits for carrying out the methods detailed herein, which comprises a pharmaceutical composition or a combination therapy described herein and instructions for use in the treatment of breast cancer.

Kits generally comprise suitable packaging. The kits may comprise one or more containers comprising any pharmaceutical composition described herein. Each component (if there is more than one component) can be packaged in separate containers or some components can be combined in one container where cross-reactivity and shelf life permit. One or more components of a kit may be sterile and/or may be contained within sterile packaging.

### Methods

Provided herein are methods of treating hormone receptor positive and HER2 negative locally advanced or metastatic breast cancer. In one embodiment, the method comprises treating hormone receptor positive and HER2 negative locally advanced or metastatic breast cancer in a patient having PIK3CA mutated, hormone receptor positive and HER2 negative locally advanced or metastatic breast cancer, by administering to the patient a combination therapy that includes inavolisib, palbociclib and an fulvestrant, which produces a statistically significant and clinically meaningful improvement to the patient compared to administering palbociclib and fulvestrant alone (not including inavolisib) to a comparable patient.

Also provided are methods of treating hormone receptor positive and HER2 negative (HR+/HER2−) locally advanced or metastatic breast cancer in a patient comprising administering a therapeutically effective amount of inavolisib, or a pharmaceutically acceptable salt thereof, palbociclib and fulvestrant.

PIK3CA mutations, found in approximately 40% of HR-positive breast cancers, are linked to tumour growth, disease progression, and treatment resistance. In one aspect, provided is a method of treating hormone receptor positive and HER2 negative locally advanced or metastatic breast cancer in a patient having PIK3CA mutant, hormone receptor positive and HER2 negative locally advanced or metastatic breast cancer, the method comprising administering to the patient a combination therapy comprising inavolisib, palbociclib and fulvestrant, wherein the combination therapy comprising inavolisib, palbociclib and fulvestrant produces a statistically significant and clinically meaningful improvement to the patient compared to administering palbociclib and fulvestrant alone (not including inavolisib) to a comparable patient. In some embodiments, the median PFS for patients treated with the combination therapy comprising inavolisib, palbociclib and fulvestrant is about 5 to 12 months longer than the median PFS for patients treated with palbociclib and fulvestrant alone (not including inavolisib), i.e., the difference between the median PFS in patients treated with the combination therapy comprising inavolisib, palbociclib and fulvestrant and the median PFS in a comparable patient treated with palbociclib and fulvestrant alone (ΔPFS) is about 5 to 10 months (e.g., 5, 6, 7, 8, 9, 10, 11 or 12 months). In some embodiments, the ΔPFS is 6 months, 7 months, 8 months, 9 months, 10 months or 11 months. In some embodiments, the ΔPFS is 6-7 months, 7-8 months, 8-9 months or 9-10 months. In some embodiments, the p-value is less than 0.0001 (<0.0001) and hazard ratio (HR) less than 0.69. In some embodiments, the HR is between 0.6 and 0.69, between 0.5 and 0.59, between 0.4 and 0.49, or between 0.3 and 0.39. In some embodiments, the objective response rate (ORR) in patients treated with the combination therapy comprising inavolisib, palbociclib and fulvestrant is about 10% to 50% higher (e.g., 20-40% higher or 25-35% higher) than a comparable patient treated with palbociclib and fulvestrant alone. In some embodiments, the clinical benefit rate (CBR) in patients treated with the combination therapy comprising inavolisib, palbociclib and fulvestrant is about 10% to 50% higher (e.g., 20-40% higher or 25-35% higher) than a comparable patient treated with palbociclib and fulvestrant alone.

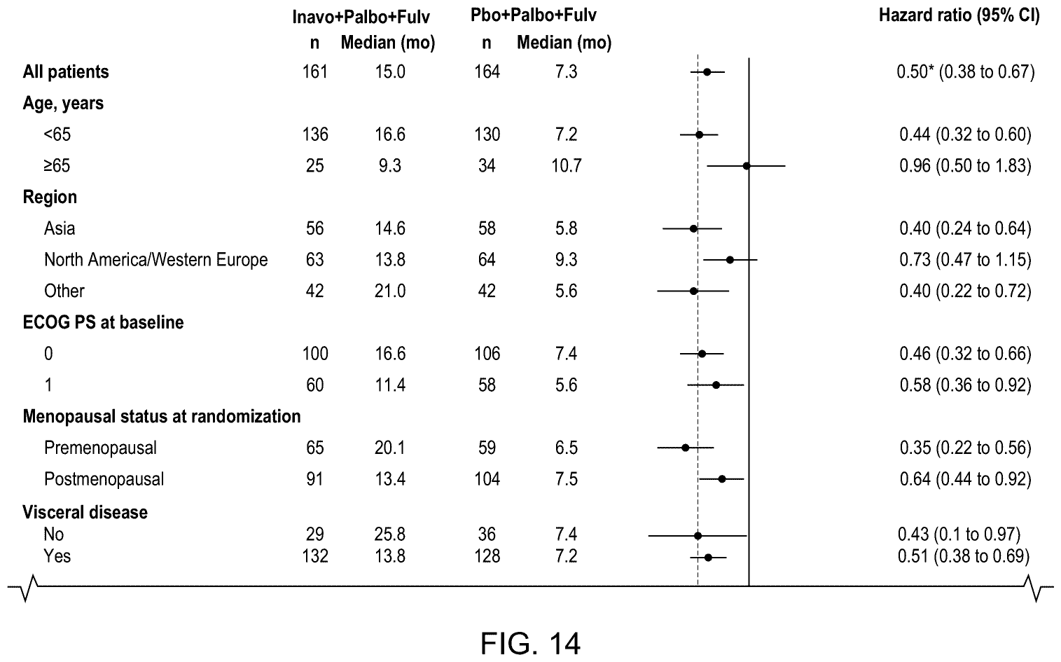

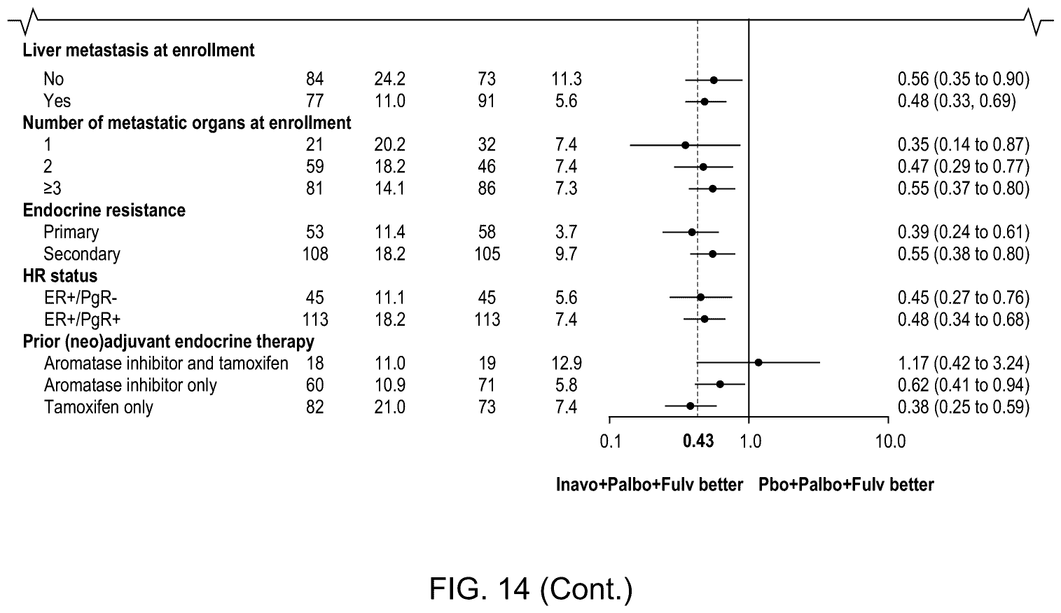

The clinical benefit to patients treated with the combination therapy comprising inavolisib, palbociclib and fulvestrant may be more or less in certain patient subgroups compared to treatment with palbociclib and fulvestrant alone to a comparable patient subgroup. See Table A1 (including 270 patients whose PIK3CA mutation-positive status were identified by a central test using the F1LCDx assay) and FIG. 14 (including all 325 patients in the INAVO120 study). For example, patients who did not have a visceral disease had a more significant increase of PFS when treated with the inavolisib combination compared to treatment with palbociclib and fulvestrant alone (ΔPFS of about 17-18 months). The patients in this study received prior adjuvant or neoadjuvant endocrine therapies (such as tamoxifen and aromatase inhibitors). Patients who receive prior tamoxifen only benefited more (ΔPFS of about 14 months) from the inavolisib combination (compared to palbociclib and fulvestrant alone) that the patients who received a prior aromatase inhibitor (ΔPFS of about 5 months). Patients who were not post-menopausal at randomization had a larger ΔPFS (about 14 months) than patients who were post-menopausal at randomization (ΔPFS about 6 months). The hormone receptor status also impacts the benefit. Patients who were ER-positive and PR-positive had ΔPFS about 11-13 months; while patients who were ER-positive and PR-negative had ΔPFS about 5 months. The majority of the patients (266 of 325) in the INAVO120 study were <65 years of age. The relatively small number of patients>=65 years of age appears to have performed poorly in the inavolisib arm; while the patients younger than 65 years of age performed better (ΔPFS about 9 months).

In some embodiments of the methods detailed herein for treating PIK3CA mutated, hormone receptor positive and HER2 negative locally advanced or metastatic breast cancer in a patient, the patient received a prior adjuvant or neoadjuvant endocrine therapy which is tamoxifen only. In some embodiments, the patient received a prior adjuvant or neoadjuvant endocrine therapy which is a aromatase inhibitor only or which includes or comprises a aromatase inhibitor. In some embodiments, the patient does not have a visceral disease (per electronic Case Report Form (eCRF)). In some embodiments, the patient has a visceral disease per eCRF). In some embodiments, the patient is not post-menopausal. In some embodiments, the patient is post-menopausal.

In some embodiments, the patient received no prior therapy for LA/mBC. In some embodiments, the patient has fasting glucose<126 mg/dL. In some embodiments, the patient has HbA_{1C}<6.0%. In some embodiments, the patient has fasting glucose<126 mg/dL and HbA_{1C}<6.0%.

In some embodiments, provided is a method of treating hormone receptor positive and HER2 negative locally advanced or metastatic breast cancer in a patient having hormone receptor positive and HER2 negative locally advanced or metastatic breast cancer with one or more PIK3CA mutations, the method comprising administering to the patient a combination therapy comprising inavolisib, palbociclib and fulvestrant, wherein said combination therapy is administered over a 28-day cycle, and wherein the combination therapy comprising inavolisib, palbociclib and fulvestrant produces a statistically significant and clinically meaningful improvement to the patient compared to administering palbociclib and fulvestrant alone to a comparable patient.

In some embodiments, provided is a method of treating hormone receptor positive and HER2 negative locally advanced or metastatic breast cancer in a patient having PIK3CA mutated, hormone receptor positive and HER2 negative locally advanced or metastatic breast cancer, the method comprising administering to the patient a combination therapy comprising a dosing regimen comprising:
a. administering inavolisib QD on days 1-28 of a first 28-day cycle;
b. administering palbociclib QD on days 1-21 of a first 28-day cycle; and
c. administering fulvestrant on days 1 and 15 of a first 28-day cycle,
   wherein administering the combination therapy comprising inavolisib, palbociclib and fulvestrant produces a statistically significant and clinically meaningful improvement to the patient compared to administering palbociclib and fulvestrant alone to a comparable patient.

In some of these embodiments, the method further comprises one or more additional 28-day cycles comprising:
a. administering inavolisib on days 1-28 of each additional 28-day cycle;
b. administering palbociclib on days 1-21 of each additional 28-day cycle; and
c. administering fulvestrant on day 1 of each additional 28-day cycle (or approximately once every 4 weeks).

In some embodiments, the patient has hormone receptor-positive, Her2-negative, locally advanced or metastatic breast cancer with a PIK3CA mutation. In some embodiments, the patient has PIK3CA mutations at one or more of positions 88, 106, 111, 118, 345, 420, 453, 542, 545, 546, 1043, 1047 and 1049. In some embodiments, the patient has PIK3CA mutations at one or more of H1047, E545, E542, Q546, N345, C420, M1043, G1049, E453, K111, G106, G118, and R88. In some embodiments, the patient has mutant PIK3CA containing one or more mutations selected from the group consisting of H1047D/I/L/N/P/Q/R/T/Y, E545A/D/G/K/L/Q/R/V, E542A/D/G/K/Q/R/V, Q546E/H/K/L/P/R, N345D/H/I/K/S/T/Y, C420R, M1043I/T/V, G1049A/C/D/R/S, E453A/D/G/K/Q/V, K111N/R/E, G106A/D/R/S/V, GI 18D, and R88Q. In some embodiments, the patient has mutant PIK3CA containing one or more mutations selected from the group consisting of E542K, E545K, Q546R, H1047L and H1047R. In some embodiments, the patient has breast cancer expressing a PIK3CA mutant selected from the group consisting of H1047D/IL/N/P/Q/R/T/Y, E545A/D/G/K/L/Q/R/V, E542A/D/G/K/Q/R/V, Q546E/H/K/L/P/R, N345D/H/I/K/S/T/Y, C420R, M1043I/T/V, G1049A/C/D/R/S, E453A/D/G/K/Q/V, K111N/R/E, G106A/D/R/S/V, G118D, and R88Q. In some embodiments, the patient has breast cancer expressing a PIK3CA mutant selected from the group consisting of E542K, E545K, Q546R, H1047L and H1047R. In some embodiments, the patient has mutant PIK3CA containing one mutation selected from the group consisting of E542K, E545K, Q546R, H1047L and H1047R, and a second mutation (e.g., a second mutation selected from E453Q/K, E726K and M1043L/I). In some embodiments, the patient has breast cancer expressing a PIK3CA mutant expressing a double mutation selected from the group consisting of E542K+E453Q/K, E542K+E726K, E542K+M1043L/I; E545K+E453Q/K, E545K+E726K, E545K+M1043L/I; H1047R+E453Q/K, and H1047R+E726K.

PIK3CA-mutated tumor status can be assessed by either central testing of blood (e.g., ctDNA) or local testing of blood (e.g., ctDNA) or tumor tissue. In some embodiments, the central test for identification of eligible PIK3CA mutations is the FoundationOne Liquid Clinical Trial Assay performed at Foundation Medicine, Inc. In some embodiments, the local tests of blood or tumor tissue is performed using a Sponsor pre-approved PCR- or NGS-based assay at a CLIA-certified or equivalent laboratory.

In one embodiment, the method includes a combination therapy comprising (i) inavolisib; (ii) fulvestrant; and (iii) palbociclib. In one embodiment, the method includes a combination therapy comprising (i) inavolisib; (ii) palbociclib; and (iii) fulvestrant administered in accordance with a dosing regimen described herein.

In a further aspect, provided is a method of inhibiting tumor growth or producing/increasing tumor regression in a patient having PIK3CA mutant/mutated, hormone receptor positive and HER2 negative locally advanced or metastatic breast cancer, the method comprising administering to the patient a combination therapy according to the methods detailed herein.

In some embodiments, estrogen receptor (ER)-positive and/or progesterone receptor (PR)-positive tumor are documented according to American Society of Clinical Oncology/College of American Pathologists (ASCO/CAP) guidelines, defined as ≥1% of tumor cells stained positive based on the most recent tumor biopsy and assessed locally.

Hormone receptor-positive (HR+) breast cancer cells have either estrogen receptor (ER) or progesterone receptor (PR) or both. In some embodiments of the methods detailed herein, the HR-positive breast cancer is ER-positive. In some embodiments, the HR-positive breast cancer is PR-positive. In some embodiments, the HR-positive breast cancer is ER-positive and PR-positive. In some embodiments, the HR-positive breast cancer is ER-positive and PR-negative.

In some embodiments HER2-negative tumor are documented according to ASCO/CAP guidelines, defined as a HER2 immunohistochemistry (IHC) score of 0 or 1+, or an IHC score of 2+ accompanied by a negative fluorescence, chromogenic, or silver in situ hybridization test indicating the absence of HER2 gene amplification, or a HER2/CEP17 ratio of <2.0 based on the most recent tumor biopsy and assessed locally.

In some embodiments, the patient is women or men≥18 years of age.

In some embodiments, the patient is an adult younger than 65 years of age.

In some embodiments, the patient is postmenopausal (e.g., a postmenopausal female). Postmenopausal female is defined by at least one of the following criteria: (1) Age≥60 years; (2) Age<60 years and 12 months of amenorrhea plus follicle-stimulating hormone and plasma estradiol levels within postmenopausal range by local laboratory assessment in the absence of oral contraceptive pills, hormone replacement therapy, or gonadotropin releasing hormone agonist or antagonist; (3) Documented bilateral oophorectomy (≥14 days prior to first treatment on Day 1 of Cycle 1 and recovery to baseline).

In some embodiments, the patient is premenopausal or perimenopausal female (i.e., not meeting the criteria for postmenopausal) and has been treated with luteinizing hormone-releasing hormone (LHRH) agonist therapy (e.g., goserelin or leuprolide) beginning at least 2 weeks prior to Day 1 of Cycle 1 and continuing for the duration of study treatment.

In some embodiments, the patient is male and has been treated with with LHRH agonist therapy (e.g., goserelin or leuprolide) beginning at least 2 weeks prior to Day 1 of Cycle 1 and continuing for the duration of study treatment.

Agents described herein can be administered in accordance with a package insert. In one embodiment of the methods described herein, agents can be administered in an effective amount as described herein. In some embodiments, palbociclib or fulvestrant, where applicable, is administered at its approved dosage by an approved route of administration.

Agents in a combination therapy detailed herein may be administered simultaneously or sequentially. Two of the triplets of a combination therapy may be administer simultaneously while the third agent may be adminstered before or after. For example, in one embodiment of the methods described herein, administration of inavolisib occurs before administration of another agent (e.g. fulvestrant). In one embodiment of the methods described herein, administration of inavolisib occurs before administration of fulvestrant and the administration of fulvestrant occurs before the administration of palbociclib. In another embodiment, inavolisib is administered prior to or concurrently with palbociclib and fulvestrant is administered thereafter.

In some embodiments, inavolisib is administered at an amount of 3, 6 or 9 mg, e.g., in one or more oral tablets. In some embodiments, inavolisib is adminstered orally at a 9 mg daily dose. In some of these embodiments, inavolisib is administered at an amount of 9 mg, e.g., in an oral tablet.

In one embodiment of the methods described herein, palbociclib is administered as an agent of the triple combination therapy described herein. In one embodiment, palbociclib is administered orally at an amount of 125 mg, 100 mg, or 75 mg. In another embodiment, palbociclib is administered orally at an amount of 125 mg. In another embodiment, palbociclib is administered orally at an amount of 100 mg. In still another embodiment, palbociclib is administered orally at an amount of 75 mg. Is such embodiments, palbociclib is administered QD on days 1-21 of each 28-day cycle. In another embodiment of the methods described herein, palbociclib is administered in accordance with a package insert. In one embodiment, palbociclib is administered orally QD on days 1-21 of each 28-day cycle at an amount described herein. In still another embodiment, the amount of palbociclib is modified (e.g. reduced) from the initial dosage. In one such embodiment, the amount of palbociclib administered is reduced from 125 mg to 100 mg and can be, in one embodiment, further reduced to 75 mg. In another embodiment, palbociclib is administered in a dosing regimen as described herein.

In some embodiments, palbociclib is administered at an amount of 125 mg, e.g., in an oral capsule or tablet.

In another embodiment of the methods described herein, fulvestrant is administered at a dose of about 500 mg. In one embodiment of the methods described herein, fulvestrant is administered in accordance with a package insert. In one embodiment, fulvestrant is administered as two separate 250 mg intramuscular injections. In another embodiment, fulvestrant is administered in a dosing regimen as described herein. In one such embodiment, fulvestrant is administered on days 1 and 15 of the first 28-day cycle and on day 1 of each subsequent 28-day cycle thereafter.

In some embodiments, fulvestrant is administered at an amount of 500 mg, e.g., by intramuscular (IM) infusion.

In one embodiment, the methods described herein include a combination therapy described herein administered according to a dosing regimen comprising one 28-day cycle. In another embodiment, the methods described herein include a combination therapy described herein administered according to a dosing regimen comprising a first 28-day cycle followed by additional 28-day cycles. In another embodiment, the methods described herein include a combination therapy described herein administered according to a dosing regimen comprising a first 28-day cycle followed by 2-10 28-day cycles. In still another embodiment, the methods described herein include a combination therapy described herein administered according to a dosing regimen comprising a first 28-day cycle followed by 2-8 28-day cycles. In one embodiment of the methods described herein, the dosing regimen comprises a first 28-day cycle followed by 2-36, 2-30, 2-24, 2-18, 2-12, 2-10, 2-8, 2-6, or 2-4 28-day cycles.

Further embodiments of the methods of treating hormone receptor positive and HER2 negative locally advanced or metastatic breast cancer are provided herein.

In one embodiment, the efficacy of the combination is measured as a function of PFS. In one such embodiment, PFS of the patient is increased by 3, 4, 5, 6, 7, 8, 9, 10, 15, 20, or more months compared to non-treatment or SOC treatment. In one embodiment, PFS is measured for at least 64 months following the first dosage of the combination therapy described herein. In another embodiment, the efficacy is measured as a function of PFS in a biomarker positive patient set (e.g. a biomarker panel as described herein including PIK3CA) comparable to a biomarker negative patient set.

In one embodiment, treatment with a combination therapy according to the methods provided herein increases a patient's OS by 3, 4, 5, 6, 7, 8, 9, 10, 15, 20 or more months comparable to non-treatment or SOC treatment. In one embodiment, treatment with a combination therapy according to the methods provided herein increases the patient's amount of ORR. In another embodiment, efficacy of response is measured as a function of DOR comparable to non-treatment or SOC treatment. In still another embodiment, efficacy of response is measured as a function of CBR comparable to non-treatment or SOC treatment.

In another embodiment, the TTP is increased in a patient following treatment with a combination therapy according to the methods provided herein. In another embodiment, the PFS is increased in a patient following treatment with a combination therapy according to the methods provided herein. In one embodiment provided herein a patient is diagnosed having a CR following treatment with a combination therapy according to the methods provided herein. In one embodiment provided herein a patient is diagnosed having a PR following treatment with a combination therapy according to the methods provided herein. In one embodiment provided herein a patient is diagnosed having SD following treatment with a combination therapy according to the methods provided herein.

In some of these embodiments, the patient has locally advanced or metastatic breast cancer (e.g., histologically or cytologically confirmed) not amenable to curative therapy (e.g., surgical or radiation therapy with curative intent).

In some embodiments, the patient has progression of disease during adjuvant endocrine treatment or within 12 months of completing adjuvant endocrine therapy (e.g., with an aromatase inhibitor or tamoxifen). In some embodiments, the patient has progression of disease during adjuvant endocrine treatment or within 12 months of completing adjuvant endocrine therapy with tamoxifen. Non-limiting examples of aromatase inhibitors include anastrozole, letrozole and exemestane. In some embodiments, wherein a CDK4/6 inhibitor was included as part of neoadjuvant or adjuvant therapy, the patient has progression event must be >12 months since completion of CDK4/6 inhibitor portion of neoadjuvant or adjuvant therapy.

In one embodiment of the methods described herein, a patient has been treated with one or more cancer therapies before administration of a combination therapy described herein. In one embodiment of the methods described herein, the prior therapy comprises fulvestrant and/or palbociclib. In another embodiment, a patient described herein has not been prior treated with fulvestrant, a PI3K inhibitor, and/or a CDK4/6 inhibitor.

In one embodiment of the methods described herein, a patient has breast cancer described herein that is resistant to one or more cancer therapies (e.g., a CDK4/6 inhibitor such as palbociclib, ribociclib, or abemaciclib). In one embodiment of the methods described herein, resistance to cancer therapy includes recurrence of cancer or refractory cancer. Recurrence may refer to the reappearance of cancer, in the original site or a new site, after treatment. In one embodiment of the methods described herein, resistance to a cancer therapy includes progression of the cancer during treatment with the anti-cancer therapy. In some embodiments of the methods described herein, resistance to a cancer therapy includes cancer that does not response to treatment. The cancer may be resistant at the beginning of treatment or it may become resistant during treatment. In some embodiments of the methods described herein, the cancer is at early stage or at late stage.

Co-administration of inavolisib with palbociclib and fulvestrant may prevent or delay development of resistance of a tumor (e.g., breast cancer) to a CDK4/6 inhibitor (e.g. palbociclib, ribociclib, or abemaciclib) or a combination of a CDK4/6 inhibitor (e.g. palbociclib, ribociclib, or abemaciclib) and fulvestrant. Thus provided is a method of preventing or delaying development of resistance of a tumor (e.g., breast cancer) to a therapy containing a CDK4/6 inhibitor (e.g. palbociclib, ribociclib, or abemaciclib), comprising administering a combination therapy detailed herein. In some embodiments, provided is a method of preventing or delaying development of resistance of a tumor (e.g., breast cancer) to a therapy containing palbociclib, comprising administering a combination therapy comprising inavolisib, palbociclib and fulvestrant. In some embodiments, the combination therapy is administered according to any methods as detailed herein.

In one embodiment, a patient described herein has been pretreated with an aromatase inhibitor (e.g., anastrozole, letrozole or exemestane) or tamoxifen prior to administration of the combination therapy described herein. In one such embodiment, the patient relapsed during prior treatment with an aromatase inhibitor or tamoxifen or otherwise demonstrated disease progression after such administration. In one such embodiment, the relapse or disease progression was observed during the first 12 months of an adjuvant endocrine therapy. In one embodiment, the prior treatment was with one or more aromatase inhibitors as described herein. In another embodiment, the prior treatment was with tamoxifen. In still another such embodiment, the prior treatment was for locally advanced or metastatic breast cancer. In one such embodiment, a patient described herein has been pretreated with letrozole, tamoxifen, anastrozole, or exemestane. In another such embodiment, a patient described herein has been treated for 3-6 years with an aromatase inhibitor or tamoxifen prior to administration of a combination therapy described herein. In another such embodiment, a patient described herein has been treated for greater than 6 years with an aromatase inhibitor or tamoxifen prior to administration of a combination therapy described herein. In still another embodiment, a patient herein is post-menopausal. In another embodiment, a patient herein has at least one measurable lesion as measured by, for example, RECIST.

In one embodiment of the methods described herein, a patient having hormone receptor positive and HER2 negative locally advanced or metastatic breast cancer as described herein may have undergone surgical treatment such as, for example, surgery that is breast-conserving (i.e., a lumpectomy, which focuses on removing the primary tumor with a margin), or more extensive (i.e., mastectomy, which aims for complete removal of all of the breast tissue) prior to administration of a combination therapy described herein. In another embodiment, a patient described herein may undergo surgical treatment following treatment with a combination therapy described herein.

Radiation therapy is typically administered post-surgery to the breast/chest wall and/or regional lymph nodes, with the goal of killing microscopic cancer cells left post-surgery. In the case of a breast conserving surgery, radiation is administered to the remaining breast tissue and sometimes to the regional lymph nodes (including axillary lymph nodes). In the case of a mastectomy, radiation may still be administered if factors that predict higher risk of local recurrence are present. In some embodiments of the methods provided herein a patient having hormone receptor positive and HER2 negative locally advanced or metastatic breast cancer as described herein may have received radiation therapy prior to administration of a combination therapy described herein. In other embodiments of the methods provided herein a patient having hormone receptor positive and HER2 negative locally advanced or metastatic breast cancer as described herein may have received radiation therapy following administration of a combination therapy described herein.

In another embodiment, the patient has not been pretreated with a PI3K inhibitor. In still another embodiment, the patient has not been pretreated with a mTOR inhibitor. In still another embodiment, the patient has not been pretreated with an AKT inhibitor. In yet another embodiment, the patient has not been previously treated with a cytotoxic chemotherapy regimen for metastatic breast cancer. In still another embodiment, a patient described herein has not been previously treated with a SERD (selective estrogen receptor degrader), including for example, fulvestrant.

Also provided herein are methods of inhibiting tumor growth or producing tumor regression in a patient described herein by administering a combination therapy described herein.

In one embodiment provided herein is a method of producing or improving tumor regression in a patient described herein by administering a combination therapy described herein.

The development of combination treatments poses challenges including, for example, the selection of agents for combination therapy that may lead to improved efficacy while maintaining acceptable toxicity. One particular challenge is the need to distinguish the incremental toxicity of the combination. In one embodiment of the methods described herein the combination therapy described herein (e.g. inavolisib, fulvestrant, and palbociclib) is administered in a dosing regimen comprising a staggered dosing schedule. In one embodiment, the combination therapy described herein (e.g. inavolisib, fulvestrant, and palbociclib) is administered simultaneously on a 28-day cycle.

In one embodiment of the methods provided herein, inavolisib is administered QD on each day of each 28-day cycle and palbociclib is administered QD on days 1-21 of each 28-day cycle. In such embodiments, fulvestrant is administered as described herein such as on day 1 and 15 of the first 28-day cycle and day 1 of each 28-day cycle thereafter.

In some embodiments, the patient has adequate hematologic and organ function within 14 days prior to initiation of study treatment.

In patients at-risk or prone to developing hyperglycemia (for example, obese or pre-diabetic patients), metformin may be administered to manage hyperglycemia in the patient. Thus in some embodiments, provided is a method of treating hormone receptor positive and HER2 negative locally advanced or metastatic breast cancer in a patient having PIK3CA mutant, hormone receptor positive and HER2 negative locally advanced or metastatic breast cancer, the method comprising administering to the patient a combination therapy comprising inavolisib, palbociclib and fulvestrant, wherein the patient has been previously treated with metformin. In some embodiments, the method comprises administering metformin, inavolisib, palbociclib and fulvestrant, wherein inavolisib, palbociclib and fulvestrant are administered according to any methods detailed herein. In some of these embodiments, the dose or regimen of metformin is adjusted to moderate, stabilize, or diminish hyperglycemia in the patient prior to administration of inavolisib. In some of these embodiments, the patient is administered from 500 mg to 2000 mg (e.g., 500 mg) metformin daily for about 15 days before administration of inavolisib. In some of these embodiments, the patient is administered from 500 mg to 2000 mg (e.g., 500 mg) metformin daily for about 15 days before administration of palbociclib and fulvestrant, followed by administration of inavolisib. In some of these embodiments, inavolisib, palbociclib and fulvestrant are administered according to a dosing regimen as detailed herein.

### Biomarkers

Breast cancer is a heterogeneous disease with many distinct subtypes as defined by molecular signatures and a diverse array of mutational profiles. In one embodiment, a patient can be tested for PIK3CA/AKT1/PTEN-alteration status. In one embodiment, a patient described herein can be tested for one or more of a phosphatase and tensin homolog (PTEN) mutation, loss of PTEN expression, a phosphatidylinositol-4,5-bisphosphate 3-kinase catalytic subunit alpha (PIK3CA) mutation, a protein kinase B alpha (AKT1) mutation, or a combination thereof. In one embodiment, the loss of PTEN expression is hemizygous or homozygous. In another embodiment, samples of patients described herein can be assessed for additional biomarkers in an effort to identify factors that may correlate with the safety and efficacy of the study treatments.

In one embodiment of the methods described herein, NGS, whole genome sequencing (WGS), other methods, or a combination thereof can be used for DNA obtained from blood samples and tumor tissue from patients described herein. Such samples may be analyzed to identify germline (e.g., BRCA1/2) and somatic alterations that are predictive of response to study drug, are associated with progression to a more severe disease state, are associated with acquired resistance to study drug, or can increase the knowledge and understanding of disease biology. In another embodiment of the methods described herein, patients described herein can have cancer characterized by activation of PI3K/Akt signaling such as activating mutations in PIK3CA or AKT1 as well as through alterations in PTEN, such as those provided herein. In another embodiment, PIK3CA/AKT1/PTEN-altered tumor status will be determined using an NGS assay (e.g., Foundation Medicine, Inc. [FMI]). Review of PIK3CA/AKT1/PTEN-altered status in archival tissue and response measures can be performed on an ongoing basis. Expression of biomarkers (e.g. PTEN) as provided herein can be measured using techniques known in the art such as, for example, immunohistochemistry (IHC).

Circulating tumor DNA (ctDNA) can be detected in the blood of cancer patients with epithelial cancers and may have diagnostic and therapeutic significance (Schwarzenbach et al. 2011). For example, the mutational status of tumor cells may be obtained through the isolation of ctDNA (Maheswaran S, et al. N Engl J Med 2008; 359:366-77), and ctDNA has been used to monitor treatment effectiveness in melanoma (Shinozaki M, et al. Clin Cancer Res 2007; 13:2068-74). Blood samples from patients described herein can be collected at screening, at time of first tumor assessment, and/or at the study completion/early termination visit. In one embodiment, the samples are used to evaluate oncogenic genetic alterations at baseline and to assess for the possible emergence of new alteration after treatment with inavolisib, palbociclib and fulvestrant.

### EXAMPLES

### Abbreviations

AEs, adverse events;
AUC₀₋₂₄, area under the concentration-time curve at 0-24 hours;
BMI: body mass index;
CDK4/6i, cyclin-dependent kinase 4/6 inhibitor;
CI, confidence interval;
Cₘₐₓ, maximum serum concentration;
CR, complete response;
ctDNA: circulating tumor DNA;
D, day;
ECOG, Eastern Cooperative Oncology Group;
GMR, geometric mean ratio;
HbA1c, glycated haemoglobin;
HER2, human epidermal growth factor receptor 2;
MAF, mutant allele frequency;
MBC, metastatic breast cancer;
MTD, maximum tolerated dose;
NCI-CTCAE, National Cancer Institute Common Terminology Criteria for Adverse Events;
NE, not evaluable;
PD, pharmacodynamics;
PrD, progressive disease;
PIK3CA, phosphatidylinositol-4,5-bisphosphate 3-kinase, catalytic subunit alpha;
PK, pharmacokinetics;
PR, partial response;
pts: patients
RECIST, Response Evaluation Criteria in Solid Tumors;
SD, stable disease;
SLD, sum of longest diameters;
TRAEs, treatment-related adverse events.

### Example 1 Phase 3 Clinical Study with Inavolisib Palbociclib Fulvestrant Combination

A multicenter, international, Phase III randomized, double-blind, placebo-controlled study (INAVO120) was conducted to evaluate the efficacy and safety of inavolisib in combination with palbociclib and fulvestrant versus placebo plus palbociclib and fulvestrant in people with PIK3CA-mutated, hormone receptor (HR)-positive, HER2-negative, locally advanced or metastatic breast cancer whose disease progressed during treatment or within 12 months of completing adjuvant endocrine therapy and who have not received prior systemic therapy for metastatic disease.

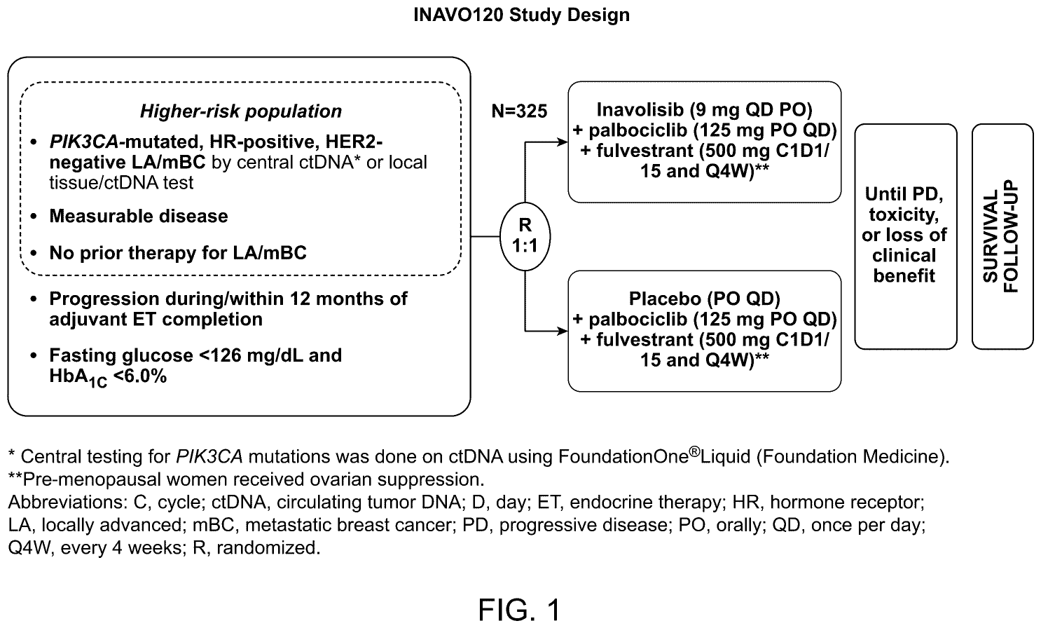

The study design is shown in FIG. 1. The multi-centered study included 325 patients, who were randomly assigned to either the investigational or control treatment arm. The PIK3CA mutation-positive status of 270 patients were identified by a central test using the F1LCDx assay performed at Foundation Medicine, Inc. (FMI). The primary endpoint is progression-free survival, as assessed by investigators, defined as the time from randomisation in the clinical trial to the time when the disease progresses, or a patient dies from any cause; 85% power, 2-sided α=0.05; primary analysis planned at ˜194 events. Secondary endpoints include overall survival (OS testing only if PFS is positive; interim OS analysis at primary PFS analysis), PFS by BICR, objective response rate (ORR), clinical benefit rate (CBR), best overall response (BOR), duration of response (DOR), patient-reported outcome (PRO), and safety.

Stratification factors include (a) Visceral Disease (Yes vs. No); (b) Endocrine Resistance (Primary vs. Secondary), defined per 4th European School of Oncology (ESO)—European Society for Medical Oncology (ESMO) International Consensus Guidelines for Advanced Breast Cancer; Primary: relapse while on the first 2 years of adjuvant endocrine therapy (ET); Secondary: relapse while on adjuvant ET after at least 2 years or relapse within 12 months of completing adjuvant ET; and (c) Region (North America/Western Europe; Asia; Other).

The study met its primary endpoint of progression-free survival (PFS), demonstrating a statistically significant and clinically meaningful improvement compared to palbociclib and fulvestrant alone. The inavolisib combination reduced the risk of disease worsening or death (progression-free survival; PFS) by over 50% compared to palbociclib and fulvestrant alone. A clear positive trend has been observed in the overall survival (OS) data available to date. Data available for other secondary endpoints showed clinically meaningful increases in ORR, DOR, and CBR at a primary analysis. The inavolisib combination was well tolerated. The most common Grade 3-4 side effects (≥5 percent) with the inavolisib combination compared to palbociclib and fulvestrant alone were neutropenia, thrombocytopenia, anemia, stomatitis and hyperglycemia.

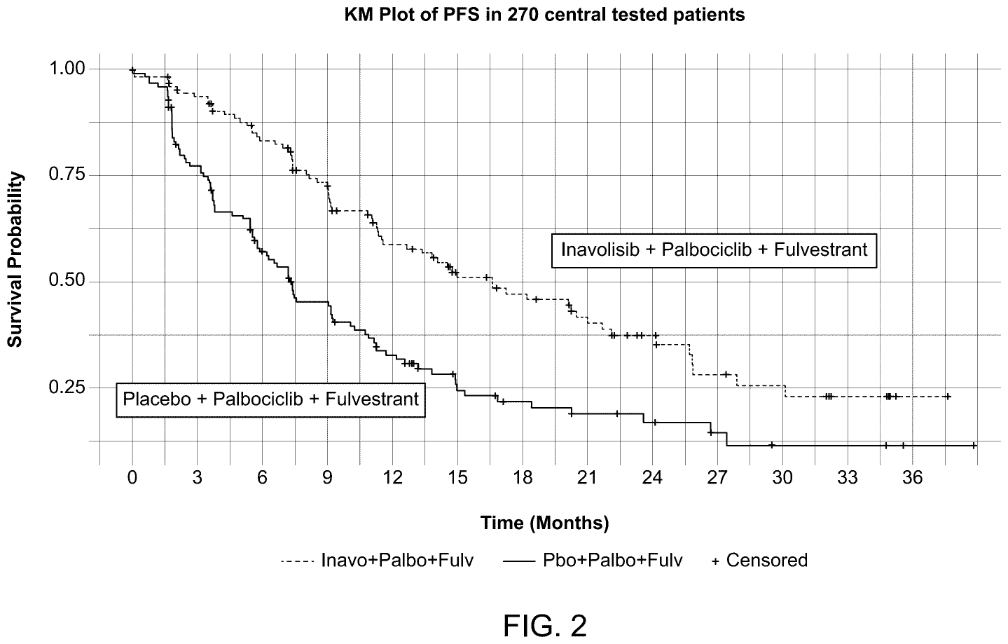

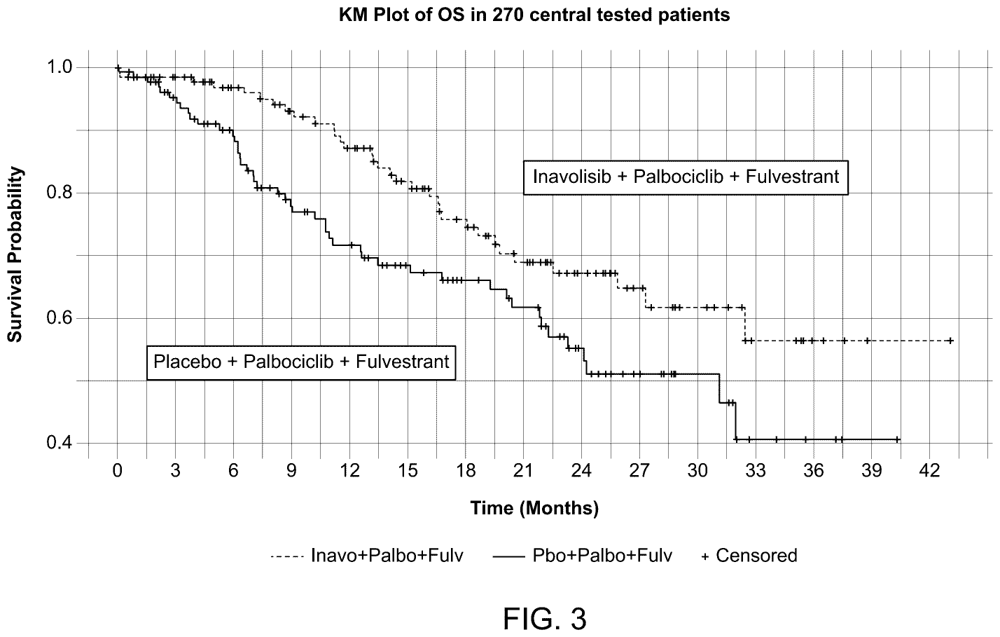

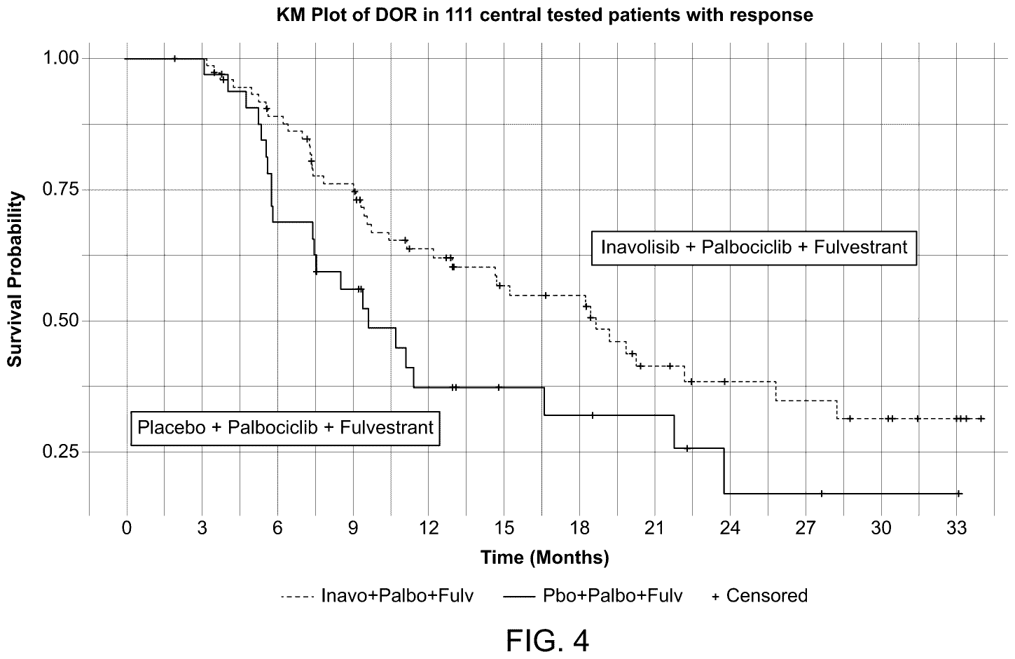

The investigator assessed PFS in the 270 patients whose PIK3CA mutation-positive status was identified by the central FMI testing and several key subgroups are listed in Table A1. FIG. 2 shows the Kaplan-Meier (KM) plot of the PFS. FIG. 3 shows the KM plot the OS trend at an interim analysis. The pre-specified boundary for significant benefit in OS (p<0.0098) was not crossed at this interim analysis. Investigator assessed ORR and CBR increased about 30% in the inavolisib arm relative to the control arm. FIG. 4 shows the KM plot of the DOR in 111 patients with response whose PIK3CA mutation-positive status was identified by the central FMI testing, showing a median DOR of about 18˜19 months for the inavolisib arm compared to a median DOR of about 9˜10 months in the control arm.

TABLE A1

| | | | Pbo + Palbo + Fulv | | | Inavo + Palbo + Fulv | | | | |
|---|---|---|---|---|---|---|---|---|---|---|
| | Total | Total | | | Median | | | Median | Hazard | |
| Baseline Risk Factors | n | Events | n | Events | (months) | n | Events | (months) | Ratio | 95% Wald CI |
| All Patients | 270 | 161 | 132 | 92 | 7.3 | 138 | 69 | 16.6 | 0.49 | (0.36, 0.67) |

Visceral disease (per eCRF)

| | | | | | | | | | | |
|---|---|---|---|---|---|---|---|---|---|---|
| No | 53 | 21 | 29 | 13 | 7.4 | 24 | 8 | 24.2 | 0.54 | (0.22, 1.31) |
| Yes | 217 | 140 | 103 | 79 | 7.2 | 114 | 61 | 14.8 | 0.46 | (0.33, 0.65) |

Liver Metastasis at Enrollment

| | | | | | | | | | | |
|---|---|---|---|---|---|---|---|---|---|---|
| Yes | 143 | 107 | 75 | 63 | 5.8 | 68 | 44 | 11.3 | 0.48 | (0.33, 0.71) |
| NO | 127 | 54 | 57 | 29 | 11.3 | 70 | 25 | 24.2 | 0.52 | (0.30, 0.88) |

Number Metastatic Organs at Enrollment

| | | | | | | | | | | |
|---|---|---|---|---|---|---|---|---|---|---|
| 1 | 41 | 21 | 24 | 15 | 7.4 | 17 | 6 | 27.9 | 0.22 | (0.07, 0.69) |
| 2 | 88 | 54 | 38 | 29 | 7.2 | 50 | 25 | 17.2 | 0.47 | (0.28, 0.81) |
| >=3 | 141 | 86 | 70 | 48 | 7.6 | 71 | 38 | 15.0 | 0.55 | (0.36, 0.84) |

Endocrine Resistance (per eCRF)

| | | | | | | | | | | |
|---|---|---|---|---|---|---|---|---|---|---|
| Primary | 88 | 62 | 45 | 36 | 3.8 | 43 | 26 | 11.6 | 0.38 | (0.22, 0.63) |
| Secondary | 181 | 99 | 86 | 56 | 9.3 | 95 | 43 | 17.2 | 0.53 | (0.36, 0.80) |
| Missing | 1 | 0 | 1 | 0 | NA | 0 | NA | NA | NA | NA |

Hormone receptor status

| | | | | | | | | | | |
|---|---|---|---|---|---|---|---|---|---|---|
| ER+/PR− | 73 | 50 | 34 | 26 | 5.9 | 39 | 24 | 11.1 | 0.53 | (0.30, 0.93) |
| ER+/PR+ | 190 | 109 | 94 | 65 | 7.4 | 96 | 44 | 20.1 | 0.44 | (0.30, 0.65) |
| Other | 7 | 2 | 4 | 1 | NA | 3 | 1 | 5.7 | 1.22 | (0.08, 19.86) |

Prior (neo)-adjuvant Endocrine Therapy

| | | | | | | | | | | |
|---|---|---|---|---|---|---|---|---|---|---|
| Aromatase inhibitor | 31 | 13 | 15 | 5 | NA | 16 | 8 | 11.0 | 1.13 | (0.36, 3.60) |
| and tamoxifen | | | | | | | | | | |
| Aromatase Inhibitor | 109 | 76 | 60 | 45 | 6.5 | 49 | 31 | 11.3 | 0.67 | (0.42, 1.07) |
| only | | | | | | | | | | |
| Tamoxifen only | 128 | 71 | 56 | 41 | 7.4 | 72 | 30 | 21.7 | 0.36 | (0.23, 0.58) |
| Missing | 2 | 1 | 1 | 1 | 1.8 | 1 | 0 | NA | 1.00 | (1.00, 1.00) |

Prior adjuvant CDK 4/6 Inhibitor

| | | | | | | | | | | |
|---|---|---|---|---|---|---|---|---|---|---|
| Yes | 3 | 2 | 1 | 0 | NA | 2 | 2 | 10.2 | NA | NA |
| No | 267 | 159 | 131 | 92 | 7.3 | 136 | 67 | 16.6 | 0.48 | (0.35, 0.66) |

Prior (neo)-adjuvant chemotherapy

| | | | | | | | | | | |
|---|---|---|---|---|---|---|---|---|---|---|
| Yes | 222 | 130 | 110 | 78 | 7.2 | 112 | 52 | 17.2 | 0.42 | (0.30, 0.60) |
| No | 48 | 31 | 22 | 14 | 11.3 | 26 | 17 | 11.3 | 0.83 | (0.40, 1.69) |

Post menopausal status at randomization

| | | | | | | | | | | |
|---|---|---|---|---|---|---|---|---|---|---|
| Not post-menopausal | 98 | 58 | 43 | 33 | 6.7 | 55 | 25 | 20.1 | 0.33 | (0.20, 0.56) |
| Post-menopausal | 168 | 101 | 89 | 59 | 7.5 | 79 | 42 | 14.1 | 0.62 | (0.42, 0.92) |
| Missing | 4 | 2 | 0 | NA | NA | 4 | 2 | 24.2 | NA | NA |

### Arms and Interventions

Experimental Arm: inavolisib+Palbociclib+Fulvestrant

Participants receive: (a) oral inavolisib on Days 1-28 of each 28-day cycle; (b) oral palbociclib on Days 1-21 of each 28-day cycle; and (c) intramuscular (IM) fulvestrant approximately every 4 weeks.

Placebo Comparator Arm: Placebo+Palbociclib+Fulvestrant

Participants receive: (a) oral placebo on Days 1-28 of each 28-day cycle; (b) oral palbociclib on Days 1-21 of each 28-day cycle; and (c) intramuscular (IM) fulvestrant approximately every 4 weeks.

### Outcome Measures

### Primary Outcome Measure:

### 1. Progression-Free Survival (PFS)

[Time Frame: From randomization to the first occurrence of disease progression or death from any cause, whichever occurs first (up to 6 years)]

The results show that inavolisib in combination with palbociclib and fulvestrant improved progression-free survival in the first-line setting.

### Secondary Outcome Measure:

### 2. Objective Response Rate (ORR)

[Time Frame: Up to 6 years]

### 3. Best Overall Response Rate (BOR)

[Time Frame: Up to 6 years]

### 4. Duration of Response (DOR)

[Time Frame: From the first occurrence of a CR or PR to the first occurrence of disease progression or death from any cause, whichever occurs first (up to 6 years)]

### 5. Clinical Benefit Rate (CBR)

[Time Frame: Up to 6 years]

### 6. Overall Survival (OS)

[Time Frame: From randomization to death from any cause (up to 6 years)]

### 7. Time to Deterioration (TTD) in Pain

[Time Frame: Treatment: Day 1 of Cycles 1-3, then Day 1 of every other cycle until treatment discontinuation. Posttreatment: Every 8 weeks for 2 years, then every 12 weeks thereafter, to end of study (up to 6 years)]

### 8. TTD in Physical Function

[Time Frame: Treatment: Day 1 of Cycles 1-3, then Day 1 of every other cycle until treatment discontinuation. Posttreatment: Every 8 weeks for 2 years, then every 12 weeks thereafter, to end of study (up to 6 years)]

### 9. TTD in Role Function

[Time Frame: Treatment: Day 1 of Cycles 1-3, then Day 1 of every other cycle until treatment discontinuation. Posttreatment: Every 8 weeks for 2 years, then every 12 weeks thereafter, to end of study (up to 6 years)]

### 10. TTD in Global Health Status

[Time Frame: Treatment: Day 1 of Cycles 1-3, then Day 1 of every other cycle until treatment discontinuation. Posttreatment: Every 8 weeks for 2 years, then every 12 weeks thereafter, to end of study (up to 6 years)]
   11. Percentage of Participants with Adverse Events
[Time Frame: From randomization through the end of study (up to 6 years)]

### 12. Plasma Concentration of Inavolisib

[Time Frame: At pre-defined intervals from baseline to the end of study (up to 6 years)]

### 13. Plasma Concentration of Palbociclib

[Time Frame: At pre-defined intervals from baseline to the end of study (up to 6 years)]

### 14. Plasma Concentration of Fulvestrant

[Time Frame: At pre-defined intervals from baseline to the end of study (up to 6 years)] Eligibility

In one embodiment of the study, patients of age 18 years or older; all sex (not gender based) are accepted. Healthy volunteers are not accepted. The target population inclusion and exclusion criteria are as follows.

### Inclusion Criteria

Confirmed diagnosis of HR+/HER2− breast cancer
Metastatic or locally advanced disease not amenable to curative therapy
Progression of disease during adjuvant endocrine treatment or within 12 months of completing adjuvant endocrine therapy with an aromatase inhibitor or tamoxifen
Receiving LHRH agonist therapy for at least 2 weeks prior to Day 1 of Cycle 1 if pre/peri-menopausal
Confirmation of biomarker eligibility (detection of specified mutation(s) of PIK3CA via specified test)
Consent to provide fresh or archival tumor tissue specimen
Measurable disease per Response Evaluation Criteria in Solid Tumors, Version 1.1; “bone-only” disease, even if considered measurable, is not eligible
Eastern Cooperative Oncology Group Performance Status of 0 or 1
Life expectancy of >6 months
Adequate hematologic and organ function within 14 days prior to initiation of study treatment

### Exclusion Criteria

Metaplastic breast cancer
Any history of leptomeningeal disease or carcinomatous meningitis
Any prior systemic therapy for metastatic breast cancer
Prior treatment with fulvestrant or any selective estrogen-receptor degrader
Prior treatment with any PI3K, AKT, or mTOR inhibitor, or any agent whose mechanism of action is to inhibit the PI3K-AKT-mTOR pathway
Type 2 diabetes requiring ongoing systemic treatment at the time of study entry; or any history of Type 1 diabetes
Known and untreated, or active CNS metastases. Patients with a history of treated CNS metastases are eligible
Active inflammatory or infectious conditions in either eye, or any eye conditions expected to require surgery during the study treatment period
Symptomatic active lung disease, or requiring daily supplemental oxygen
History of inflammatory bowel disease or active bowel inflammation
Anti-cancer therapy within 2 weeks before study entry
Investigational drug(s) within 4 weeks before randomization
Prior radiotherapy to >=25% of bone marrow, or hematopoietic stem cell or bone marrow transplantation
Chronic corticosteroid therapy or immunosuppressants
Pregnant, lactating, or breastfeeding, or intending to become pregnant during the study or within 60 days after the final dose of study treatment
Major surgical procedure, or significant traumatic injury, within 28 days prior to Day 1 of Cycle 1

### Example 2 INAVO120 Study Additional Analysis

Additional analysis were performed on the results from the INAVO120 phase 3 study. In addition to the efficacy endpoints including progression-free survival (PFS), overall survival (OS), objective response rate (ORR), best overall response (BOR), clinical benefit rate (CBR) and duration of response (DOR), time from randomization to end or discontinuation of next-line treatment, or death from any cause (proxy for PFS2), time from randomization to first subsequent chemotherapy after treatment discontinuation, and time to confirmed clinical meaningful deterioration (TTCD) were also assessed. Safety endpoints and patient-reported outcomes (PRO) endpoints were also evaluated.

Statistical methods: For efficacy endpoints and TTCD, hazard ratios were estimated using a Cox proportional hazard model with 95% CI and Kaplan-Meier methodology was used to estimate the medians with the Brookmeyer-Crowley method used for the 95% CI. The clinical cutoff date (CCOD) was 29 Sep. 2023; the median follow-up was 21.3 months.

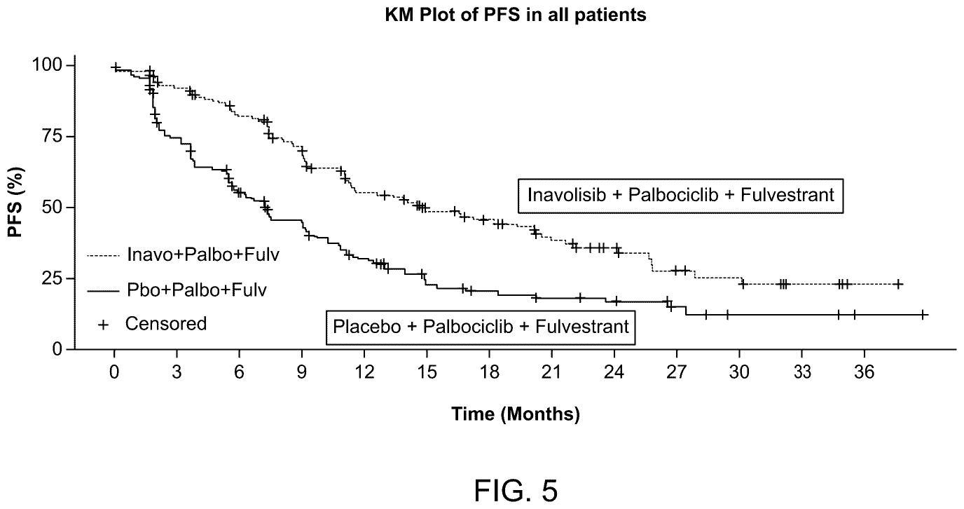

INAVO120 met its primary endpoint, with a statistically significant and clinically meaningful improvement in PFS (15.0 months versus 7.3 months; hazard ratio, 0.43 [95% CI 0.32-0.59]; p<0.0001). See FIG. 5.

Inavolisib with palbociclib and fulvestrant was associated with sustained benefit beyond disease progression, demonstrating a delayed need for subsequent therapy (Δ 8.9 months), including chemotherapy (NE versus 15.0 months), and supporting the clinical benefit of the inavolisib-based therapy.

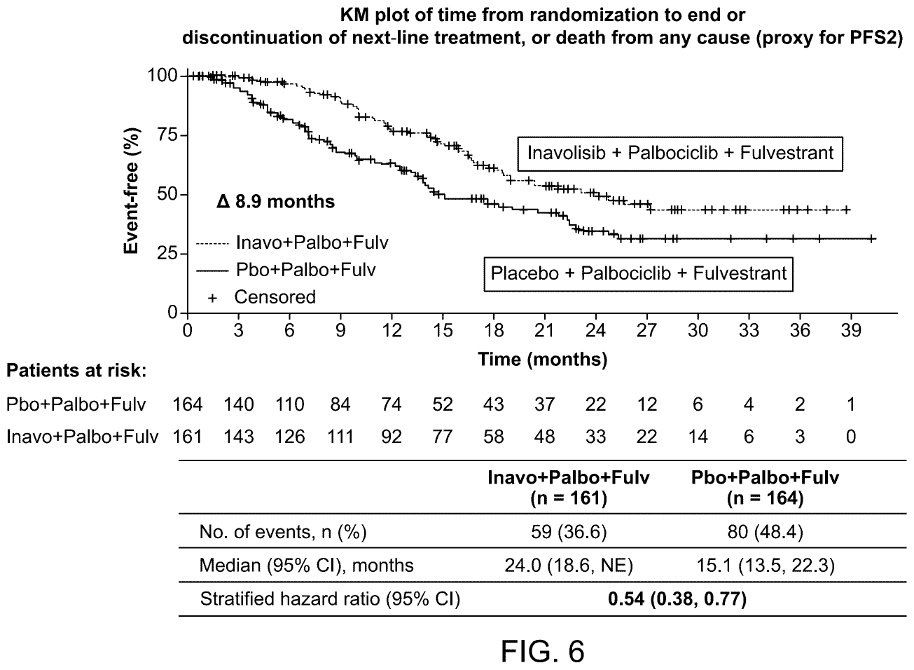

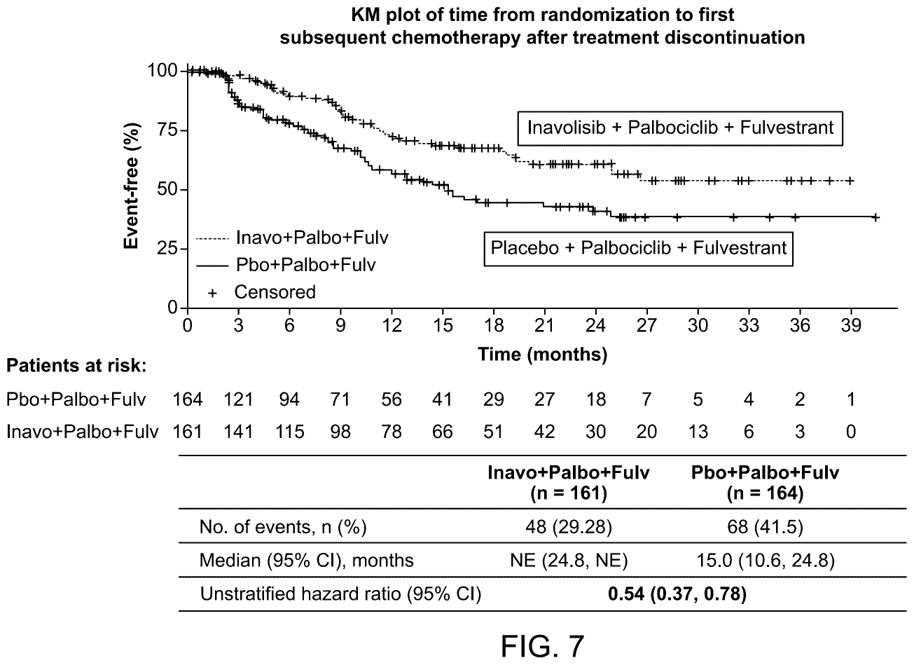

The median time from randomization to end or discontinuation of next-line treatment, or death from any cause, which is a proxy for PFS2 (time from randomization to next progression after discontinuing study treatment for progressive disease, or death from any cause) was 24.0 (95% CI 18.6—NE) months for the inavolisib arm and 15.1 (95% CI 13.5-22.3) months for the placebo arm (Δ 8.9 months), with a stratified hazard ratio of 0.54 (95% CI 0.38-0.77). See FIG. 6. The median time from randomization to first subsequent chemotherapy after treatment discontinuation was not evaluable (95% CI 24.8—NE) months for the inavolisib arm and 15.0 (95% CI 10.6-24.8) months for the placebo arm, with a stratified hazard ratio of 0.54 (95% CI 0.37-0.78). See FIG. 7.

The post-progression therapies are listed in the table below.

| | Inavo + | | Pbo + | |
|---|---|---|---|---|
| | Palbo + Fulv | | Palbo + Fulv | |
| Patients, n/N (%) | (n = 161) | | (n = 164) | |
| Discontinued treatment | 93/161 | (57.8) | 115/164 | (70.1) |
| No subsequent therapy - death | 12/161 | (7.5) | 19/164 | (11.6) |
| Received subsequent therapy* | 65/161 | (40.4) | 82/164 | (50.0) |
| Chemotherapy (any) | 40/65 | (61.5) | 60/82 | (73.2) |
| Capecitabine | 21/65 | (32.3) | 29/82 | (35.4) |
| ADC (any) | 0 | | 1/82 | (1.2) |
| PI3K inhibitor (any) | 2/65 | (3.1) | 21/82 | (25.6) |
| Alpelisib | 2/65 | (3.1) | 14/82 | (17.1) |
| mTOR kinase inhibitor (any) | 8/65 | (12.3) | 6/82 | (7.3) |
| Everolimus | 8/65 | (12.3) | 6/82 | (7.3) |
| CDK4/6 inhibitor (any) | 8/65 | (12.3) | 5/82 | (6.1) |
| Ribociclib | 1/65 | (1.5) | 5/82 | (6.1) |
| Abemaciclib | 3/65 | (4.6) | 0 | |
| Other (any)^{†} | 13/65 | (20.0) | 10/82 | (12.2) |

*One patient had a treatment unrelated to breast cancer (cytarabine) in the placebo arm;
^{†}Includes less common HR+ therapies, such as PARP inhibitors and HER2-targeted therapies.

### Safety Endpoints

Key selected adverse events including hyperglycemia, diarrhea, rash, and stomatitis/mucosal inflammation were assessed using grouped terms; severity reported per National Cancer Institute Common Terminology Criteria for Adverse Events (CTCAE) version 5.0. The number of patients with at least one AE in the inavolisib and the placebo arms, and the time to onset of first occurrence of the AE (if an AE was resolved and recurred in the same patient it is not included a second time in this dataset) in the inavolisib arm are as follows:

| | | | Time to onset of first |
|---|---|---|---|
| | Inavolisib | Placebo | occurrence of the AE |
| AEs | n | n | Median (range), days |
| Hyperglycemia | 95 | 14 | 7.0 (2.0-955.0) |
| Diarrhea | 78 | 26 | 15.0 (2.0-602.0) |
| Rash | 41 | 28 | 29.0 (1.0-952.0) |
| Stomatitis/mucosal | 83 | 43 | 13.0 (1.0-610.0) |
| inflammation | | | |

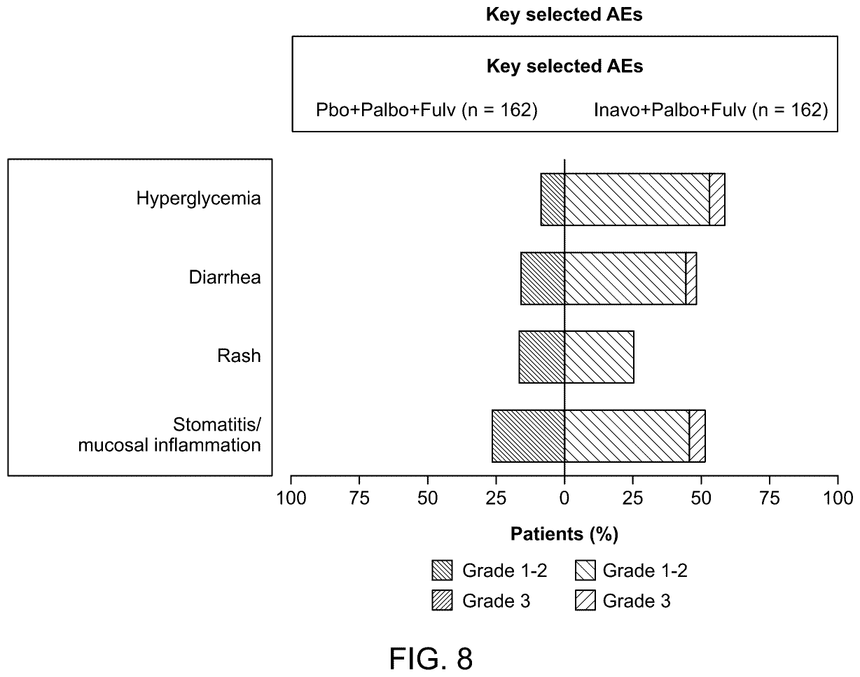

A graphical representation is shown in FIG. 8. The majority of key selected AEs had resolved (‘resolution’ was per investigator decision) by the CCOD; some patients were enrolled close to the CCOD and AE follow-up is ongoing for these patients.

The number of patients in the inavolisib arm who experienced inavolisib treatment interruption, reduction, and discontinuation due to key selected AEs are shown in the table below.

| Patients, n (%) | Interruption | | Reduction | | Discontinuation |
|---|---|---|---|---|---|
| Hyperglycemia | 44 | (27.2) | 4 | (2.5) | 1 (0.6)* |
| Diarrhea | 11 | (6.8) | 2 | (1.2) | 0 |
| Rash | 2 | (1.2) | 1 | (0.6) | 0 |
| Stomatitis/mucosal | 16 | (9.9) | 6 | (3.7) | 1 (0.6) |
| inflammation | | | | | |

*One patient discontinued due to an AE of Type 2 diabetes in the inavolisib arm, which was not captured under hyperglycemia.

The discontinuation rate of inavolisib due to any AE was 6.2%. Inavolisib discontinuations for hyperglycemia, diarrhea, rash, and stomatitis/mucosal inflammation were low, confirming the manageable safety and tolerability profile of inavolisib.

The patients received concomitant medications for adverse events. The number of patients receiving concomitant medications for key selected AEs as well as the most common concomitant medications per AE are listed below.

| | | |
|---|---|---|
| | Inavolisib Arm | Placebo Arm |
| Patients, n/N (%) | (n = 162) | (n = 162) |

Received ≥1 concomitant
medication for:

| Hyperglycemia | 66/162 | (40.7) | 1/162 | (0.6) |
|---|---|---|---|---|
| Diarrhea | 46/162 | (28.4) | 6/162 | (3.7) |
| Rash | 26/162 | (16.0) | 19/162 | (11.7) |
| Stomatitis/mucosal inflammation | 69/162 | (42.6) | 26/162 | (16.0) |
| Most common concomitant | | | | |
| medications per AE: | | | | |
| Metformin: hyperglycemia | 62/66 | (93.9) | 1/1 | (100) |
| Loperamide: diarrhea | 38/46 | (82.6) | 6/6 | (100) |
| Hydrocortisone (topical): rash | 5/26 | (19.2) | 3/19 | (15.8) |
| Steroid (mouthwash): | 42/69 | (60.9) | 12/26 | (46.1) |
| stomatitis/mucosal inflammation | | | | |

| | | |
|---|---|---|
| Prophylactic use | (20) | (14.2) |

### Patient-Reported Outcomes Endpoints

Patient-reported outcomes endpoints were assessed at Day 1 of Cycles 1-3 then Day 1 of every other Cycle thereafter (5, 7, 9, etc.), at treatment discontinuation, and every 3 months during survival follow-up. Baseline completion rates in both arms were >90% for all assessments; post-baseline rates in both arms remain >80% through Cycle 15; <50% intent-to-treat sample remaining at Cycle 9 in the inavolisib arm versus Cycle 5 in the placebo arm.

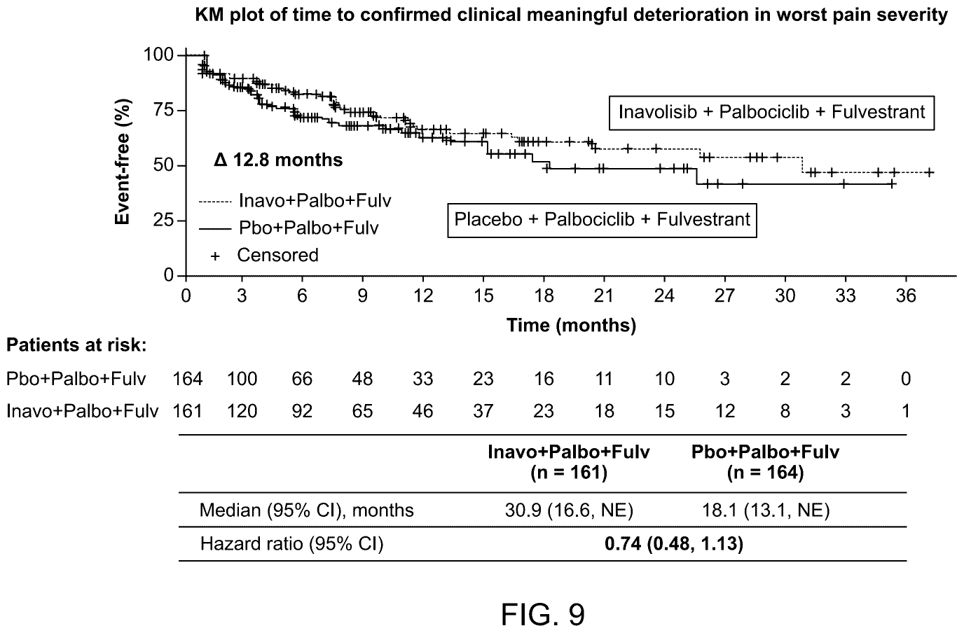

Patients in the inavolisib arm experienced a longer duration of time without confirmed, clinically meaningful worsening pain severity than patients in the placebo arm. The time to confirmed clinical meaningful deterioration in worst pain severity was assessed using the brief pain inventory-short form (BPI-SF). The median time was 30.9 (95% CI 16.6—NE) months for the inavolisib arm and 18.1 (95% CI 13.1—NE) months for the placebo arm (Δ 12.8 months), with a hazard ration of 0.74 (95% CI 0.48-1.13). See FIG. 9.

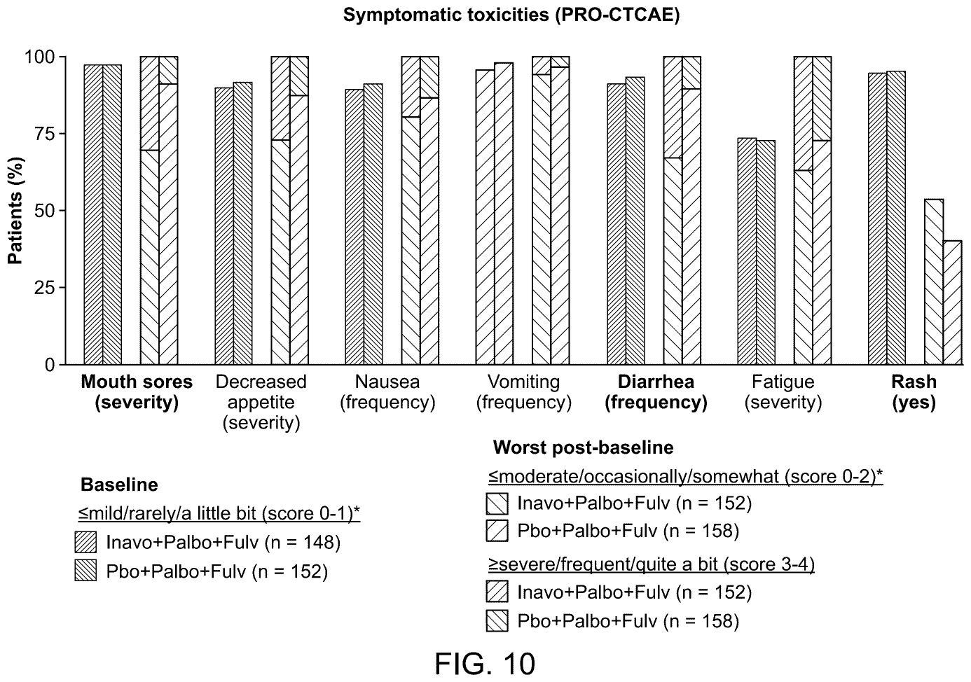

Symptomatic toxicities were assessed by patient-reported outcomes using the Common Terminology Criteria for Adverse Events (CTCAE). Most patients reported worst post-baseline symptomatic toxicities at moderate levels or less. Diarrhea, mouth sores, and rash were experienced at higher (worse) levels by patients in the inavolisib arm than the placebo arm. The percent of patients based on the baseline and worst post-baseline PRO-CTCAE scores are shown in FIG. 10.

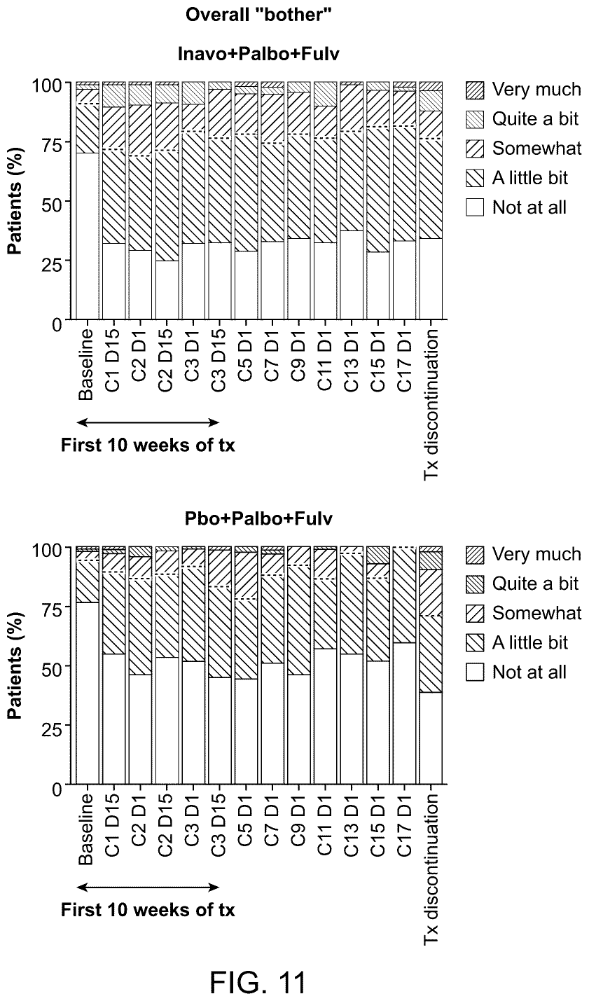

When asked “In the last 7 days, how bothered were you by the side effect(s) of your treatment?” most patients reported overall “bother” from treatment as “not at all” or “a little bit”. The percent of patients in each “bother” category during the course of the treatment are shown in FIG. 11.

Patient-reported outcomes data suggest patients receiving inavolisib in addition to fulvestrant and palbociclib experienced a longer median time to deterioration in pain severity (A 12.8 months), and maintained day-to-day functioning and health-related quality of life (HRQoL) while on treatment with little increased treatment burden. Patients in both arms maintained baseline levels of HRQoL, physical functioning, and role functioning. Adding inavolisib had no detrimental impact on patients' physical functioning, role functioning, and HRQoL.

### Example 3 ITOVEBI™ (Inavolisib) Prescribing Information

A New Drug Application was filed at the US FDA based on the results from the INAVO120 study. The prescribing information in a version of the draft label prior to approval is included below.

### Full Prescribing Information

### 1 Indications and Usage

ITOVEBI, in combination with palbociclib and fulvestrant, is indicated for the treatment of adults with endocrine-resistant, PIK3CA-mutated, hormone receptor (HR)-positive, human epidermal growth factor receptor 2 (HER2)-negative, locally advanced or metastatic breast cancer, as detected by an FDA-approved test, following recurrence on or after completing adjuvant endocrine therapy [see Clinical Studies (14.1)].

### 2 Dosage and Administration

### 2.1 Patient Selection

Select patients for the treatment of HR-positive, HER2-negative, locally advanced or metastatic breast cancer with ITOVEBI based on the presence of one or more PIK3CA mutations in plasma specimens [see Clinical Studies (14.1)].

Information on FDA-approved tests for the detection of PIK3CA mutations in breast cancer is available at: www.fda.gov/companiondiagnostics.

### 2.2 Recommended Evaluation Before Initiating ITOVEBI

Evaluate fasting plasma glucose (FPG)/blood glucose (FBG) and hemoglobin Aic (HbA_{1C}) and optimize blood glucose prior to starting ITOVEBI and at regular intervals during treatment [see Warnings and Precautions (5.1)].

### 2.3 Recommended Dosage

The recommended dosage of ITOVEBI is 9 mg taken orally once daily, with or without food, until disease progression or unacceptable toxicity.

Advise patients to take ITOVEBI at approximately the same time each day.
Swallow ITOVEBI tablet(s) whole. Do not chew, crush, or split prior to swallowing.
If a patient misses a dose, instruct the patient to take the missed dose as soon as possible within 9 hours. After more than 9 hours, instruct the patient to skip the dose and take the next dose at the scheduled time.

If a patient vomits a dose, instruct patients not to take an additional dose on that day and resume the usual dosing schedule the next day.

Administer ITOVEBI in combination with palbociclib and fulvestrant. The recommended dosage of palbociclib is 125 mg taken orally once daily for 21 consecutive days followed by 7 days off treatment to comprise a cycle of 28 days. Refer to the Full Prescribing Information for palbociclib and fulvestrant for dosing information.

For premenopausal and perimenopausal women, administer a luteinizing hormone-releasing hormone (LHRH) agonist in accordance with local clinical practice.

For men, consider administering an LHRH agonist in accordance with local clinical practice.

### 2.4 Dosage Modifications for Adverse Reactions

The recommended dose reduction levels of ITOVEBI for adverse reactions are listed in Table 1. Permanently discontinue ITOVEBI if patients are unable to tolerate the second dose reduction.

TABLE 1
Dose Reduction for Adverse Reactions

| Dose Level | Dose and Schedule |
|---|---|
| Recommended starting dose | 9 mg daily |
| First dose reduction | 6 mg daily |
| Second dose reduction | 3 mg daily |

The recommended dosage modifications of ITOVEBI for adverse reactions are summarized in Table 2.

TABLE 2
Recommended Dosage Modifications for Adverse Reactions

| Adverse Reaction | Severity | Dosage Modification |
|---|---|---|
| Hyperglycemia^{a} | Fasting glucose levels | No adjustment of ITOVEBI required. |
| [see Warnings and | (FPG or FBG) | Consider dietary modifications and ensure adequate |
| Precautions (5.1)] | >ULN to 160 mg/dL | hydration. |
| | (>ULN-8.9 mmol/L) | Initiate or intensify oral anti-hyperglycemic |
| | | medications for patients with risk factors for |
| | | hyperglycemia. |
| | Fasting glucose levels | Withhold ITOVEBI until FPG or FBG ≤160 mg/dL |
| | >160 to 250 mg/dL | (≤8.9 mmol/L). |
| | (>8.9-13.9 mmol/L) | Initiate or intensify anti-hyperglycemic medications. |
| | | Resume ITOVEBI at the same dose level. |
| | | If FPG or FBG persists >200-250 mg/dL (>11.1-13.9 |
| | | mmol/L) for 7 days under appropriate anti- |
| | | hyperglycemic treatment, consider consultation |
| | | with a healthcare professional experienced in |
| | | the treatment of hyperglycemia. |
| | Fasting glucose levels | Withhold ITOVEBI. |
| | >250 to 500 mg/dL | Initiate or intensify anti-hyperglycemic medications. |
| | (>13.9-27.8 mmol/L) | Administer appropriate hydration if required. |
| | | If FPG or FBG decreases to ≤160 mg/dL (≤8.9 mmol/L) |
| | | within 7 days, resume ITOVEBI at the same dose level. |
| | | If FPG or FBG decreases to ≤160 mg/dL (≤8.9 mmol/L) |
| | | in ≥8 days, resume ITOVEBI at one lower dose level. |
| | | If FPG or FBG >250 to 500 mg/dL (>13.9-27.8 |
| | | mmol/L) recurs within 30 days, withhold |
| | | ITOVEBI until FPG or FBG decreases to ≤160 |
| | | mg/dL (≤8.9 mmol/L). Resume ITOVEBI at one |
| | | lower dose level. |
| | Fasting glucose levels | Withhold ITOVEBI. |
| | >500 mg/dL | Initiate or intensify anti-hyperglycemic medications. |
| | (>27.8 mmol/L) | Assess for volume depletion and ketosis and |
| | | administer appropriate hydration. |
| | | If FPG or FBG decreases to ≤160 mg/dL (≤8.9 mmol/L), |
| | | resume ITOVEBI at one lower dose level. |
| | | If FPG or FBG >500 mg/dL (>27.8 mmol/L) recurs |
| | | within 30 days, permanently discontinue ITOVEBI. |
| Stomatitis | Grade 1^{b} | No adjustment of ITOVEBI required. |
| [see Warnings and | | Initiate or intensify appropriate medical therapy |
| Precautions (5.2)] | | (e.g., corticosteroid-containing mouthwash) as |
| | | clinically indicated. |
| | Grade 2^{b} | Withhold ITOVEBI until recovery to Grade ≤1. |
| | | Initiate or intensify appropriate medical therapy. |
| | | Resume ITOVEBI at the same dose level. |
| | | For recurrent Grade 2 stomatitis, withhold ITOVEBI |
| | | until recovery to Grade ≤1, then resume ITOVEBI at |
| | | one lower dose level. |
| | Grade 3^{b} | Withhold ITOVEBI until recovery to Grade ≤1. |
| | | Initiate or intensify appropriate medical therapy. |
| | | Resume ITOVEBI at one lower dose level. |
| | Grade 4^{b} | Permanently discontinue ITOVEBI. |
| Diarrhea | Grade 1^{b} | No adjustment of ITOVEBI required. |
| [see Warnings and | | Initiate appropriate medical therapy and monitor |
| Precautions (5.3)] | | as clinically indicated. |
| | Grade 2^{b} | Withhold ITOVEBI until recovery to Grade ≤1, then |
| | | resume ITOVEBI at the same dose level. |
| | | Initiate or intensify appropriate medical therapy |
| | | and monitor as clinically indicated. |
| | | For recurrent Grade 2 diarrhea, withhold ITOVEBI |
| | | until recovery to Grade ≤1, then resume ITOVEBI |
| | | at one lower dose level. |
| | Grade 3^{b} | Withhold ITOVEBI until recovery to Grade ≤1, then |
| | | resume ITOVEBI at one lower dose level. |
| | | Initiate or intensify appropriate medical therapy |
| | | and monitor as clinically indicated. |
| | Grade 4^{b} | Permanently discontinue ITOVEBI. |
| Hematologic | Grade 1, 2, or 3^{b} | No adjustment of ITOVEBI required. |
| Toxicities | | Monitor complete blood count and for signs or symptoms |
| [see Adverse | | of hematologic toxicities as clinically indicated. |
| Reactions (6.1)] | Grade 4^{b} | Withhold ITOVEBI until recovery to Grade ≤2. |
| | | Resume ITOVEBI at the same dose level or reduce to |
| | | one lower dose level as clinically indicated. |
| Other Adverse | Grade 1^{b} | No adjustment of ITOVEBI required. |
| Reactions | Grade 2^{b} | Consider withholding ITOVEBI, if clinically |
| [see Adverse | | indicated, until recovery to Grade ≤1. |
| Reactions (6.1)] | | Resume ITOVEBI at the same dose level. |
| | Grade 3 (first event)^{b} | Withhold ITOVEBI until recovery to Grade ≤1. |
| | | Resume ITOVEBI at the same dose level or one |
| | | lower dose level based on clinical evaluation. |
| | Grade 3 (recurrent)^{b} | Withhold ITOVEBI until recovery to Grade ≤1. |
| | | Resume ITOVEBI at one lower dose level. |
| | Grade 4^{b} | Permanently discontinue ITOVEBI. |

^{a}Before initiating treatment with ITOVEBI, test FPG or FBG, and HbA_{1C} levels, and optimize plasma/blood glucose levels in all patients. After initiating treatment with ITOVEBI, monitor FPG or FBG levels based on the recommended schedule, and as clinically indicated [see Warnings and Precautions (5.1)].
^{b}Based on CTCAE version 5.0.

### 2.5 Dosage Modification for Moderate Renal Impairment

The recommended starting dosage of ITOVEBI for patients with moderate renal impairment (eGFR 30 to <60 mL/min based on CKD-EPI) is 6 mg orally once daily [see Use in Specific Populations (8.6) and Clinical Pharmacology (12.3)].

### 3 Dosage Forms and Strengths

### Tablets:

3 mg: red and round convex-shaped with an “INA 3” debossing on one side.
9 mg: pink and oval-shaped with an “INA 9” debossing on one side.

### 4 Contraindications

None.

### 5 Warnings and Precautions

### 5.1 Hyperglycemia

Severe hyperglycemia can occur in patients treated with ITOVEBI.

Increased fasting glucose occurred in 85% of patients treated with ITOVEBI, including 22% of patients with Grade 2 (FPG>160 to 250 mg/dL), 12% with Grade 3 (FPG>250 to 500 mg/dL), and 0.6% with Grade 4 (FPG>500 mg/dL) events.

In INAVO120, 46% (74/162) of patients who received ITOVEBI were treated with oral anti-hyperglycemic medications and 7% (11/162) were treated with insulin to manage increased fasting glucose. In patients who experienced increased fasting glucose of >160 mg/dL, 96% (52/54) had an improvement in fasting glucose of at least one grade level with a median time to improvement from the first event of 8 days (range: 2 to 43 days).

Among patients with hyperglycemia, the median time to first onset was 7 days (range: 2 to 955 days). Hyperglycemia led to dose interruption in 28%, to dose reduction in 2.5%, and to discontinuation of ITOVEBI in 1.2% of patients.

The safety of ITOVEBI in patients with Type 1 diabetes mellitus, or Type 2 diabetes mellitus requiring ongoing anti-hyperglycemic treatment have not been studied.

Before initiating treatment with ITOVEBI, test fasting glucose levels (FPG or FBG), HbA_{1C} levels, and optimize fasting glucose.

After initiating treatment with ITOVEBI, or in patients who experience hyperglycemia after initiating treatment with ITOVEBI, monitor or self-monitor fasting glucose levels once every 3 days for the first week (Day 1 to 7), then once every week for the next 3 weeks (Day 8 to 28), then once every 2 weeks for the next 8 weeks, then once every 4 weeks thereafter, and as clinically indicated. Monitor HbA_{1C} every 3 months and as clinically indicated.

Manage hyperglycemia with anti-hyperglycemic medications as clinically indicated. During treatment with anti-hyperglycemic medication, continue monitoring fasting glucose levels. Patients with a history of well-controlled Type 2 diabetes mellitus may require intensified anti-hyperglycemic treatment and close monitoring of fasting glucose levels.

Consider consultation with a healthcare professional experienced in the treatment of hyperglycemia, and initiation of fasting glucose monitoring at home for patients who have risk factors for hyperglycemia or who experience hyperglycemia. Advise patients of the signs and symptoms of hyperglycemia and counsel patients on lifestyle changes.

Based on the severity of the hyperglycemia, ITOVEBI may require dose interruption, reduction, or discontinuation [see Dosage and Administration (2.4)].

### 5.2 Stomatitis

Severe stomatitis can occur in patients treated with ITOVEBI.

Stomatitis occurred in 51% of patients treated with ITOVEBI in combination with palbociclib and fulvestrant, including Grade 3 events in 6% of patients. The median time to first onset was 13 days (range: 1 to 610 days).

Stomatitis led to interruption of ITOVEBI in 10%, to dose reduction in 3.7%, and to discontinuation of ITOVEBI in 0.6% of patients.

In patients who received ITOVEBI in combination with palbociclib and fulvestrant, 38% used a mouthwash containing corticosteroid for management or prophylaxis of stomatitis.

Monitor patients for signs and symptoms of stomatitis. Withhold, reduce dose, or permanently discontinue ITOVEBI based on severity [see Dosage and Administration (2.4)].

### 5.3 Diarrhea

Severe diarrhea including dehydration and acute kidney injury can occur in patients treated with ITOVEBI.

Diarrhea occurred in 48% of patients treated with ITOVEBI in combination with palbociclib and fulvestrant, including Grade 3 events in 3.7% of patients. The median time to first onset was 15 days (range: 2 to 602 days). Anti-diarrheal medicines were used in 28% (46/162) of patients who received ITOVEBI in combination with palbociclib and fulvestrant to manage symptoms. Dose interruptions were required in 7% of patients, and dose reductions occurred in 1.2% of patients.

Monitor patients for signs and symptoms of diarrhea. Advise patients to increase oral fluids and start anti-diarrheal treatment at the first sign of diarrhea while taking ITOVEBI. Withhold, reduce dose, or permanently discontinue ITOVEBI based on severity [see Dosage and Administration (2.4)].

### 5.4 Embryo-Fetal Toxicity

Based on findings in animals and its mechanism of action, ITOVEBI can cause fetal harm when administered to a pregnant woman [see Clinical Pharmacology (12.1)]. In an animal reproduction study, oral administration of inavolisib to pregnant rats during the period of organogenesis caused adverse developmental outcomes, including embryo-fetal mortality, structural abnormalities, and alterations to growth at maternal exposures approximately equivalent to the human exposure at the recommended dose of 9 mg/day based on area under the curve (AUC).

Advise pregnant women and females of reproductive potential of the potential risk to a fetus. Advise females of reproductive potential to use effective non-hormonal contraception during treatment with ITOVEBI and for 1 week after the last dose. Advise male patients with female partners of reproductive potential to use effective contraception during treatment with ITOVEBI and for 1 week after the last dose [see Use in Specific Populations (8.1 and 8.3)].

ITOVEBI is used in combination with palbociclib and fulvestrant. Refer to the Full Prescribing Information of palbociclib and fulvestrant for pregnancy and contraception information.

### 6 Adverse Reactions

The following adverse reactions are discussed in greater detail in other sections of the label:
Hyperglycemia [see Warnings and Precautions (5.1)]
Stomatitis [see Warnings and Precautions (5.2)]
Diarrhea [see Warnings and Precautions (5.3)]

### 6.1 Clinical Trial Experience

Because clinical trials are conducted under widely varying conditions, adverse reaction rates observed in the clinical trials of a drug cannot be directly compared to rates in the clinical trials of another drug and may not reflect the rates observed in practice.

### Locally Advanced or Metastatic Breast Cancer

### INAVO120

The safety of ITOVEBI was evaluated in a randomized, double-blind, placebo-controlled study (INAVO120) in 324 patients with PIK3CA-mutated, HR-positive, HER2− negative, locally advanced or metastatic breast cancer [see Clinical Studies (14.1)].

Patients received either ITOVEBI 9 mg (n=162) or placebo (n=162) with palbociclib and fulvestrant. The median duration of treatment with ITOVEBI was 9 months (range: 0 to 39 months) in the ITOVEBI with palbociclib and fulvestrant arm.

Serious adverse reactions occurred in 24% of patients who received ITOVEBI with palbociclib and fulvestrant. Serious adverse reactions in ≥1% of patients included anemia (1.9%), diarrhea (1.2%), and urinary tract infection (1.2%).

Fatal adverse reactions occurred in 3.7% of patients who received ITOVEBI with palbociclib and fulvestrant, including (0.6% each) acute coronary syndrome, cerebral hemorrhage, cerebrovascular accident, COVID-19 infection, and gastrointestinal hemorrhage.

Permanent discontinuation of ITOVEBI due to an adverse reaction occurred in 6% of patients. Adverse reactions which resulted in permanent discontinuation of ITOVEBI included hyperglycemia (1.2%), and (0.6% each) stomatitis, gastric ulcer, intestinal perforation, anal abscess, increased ALT, decreased weight, bone pain, musculoskeletal pain, transitional cell carcinoma, and acute kidney injury.

Dosage interruptions of ITOVEBI due to an adverse reaction occurred in 69% of patients. Adverse reactions which required dosage interruption in ≥2% of patients included hyperglycemia (28%), neutropenia (23%), COVID-19 infection (16%), stomatitis (10%), diarrhea (7%), thrombocytopenia (4.9%), anemia (4.3%), upper respiratory tract infection (4.3%), decreased white blood cell count (3.7%), pyrexia (3.1%), nausea (2.5%), and fatigue (2.5%).

Dose reductions of ITOVEBI due to adverse reactions occurred in 14% of patients. Adverse reactions which required dose reduction of ITOVEBI in ≥2% of patients were stomatitis (3.7%) and hyperglycemia (2.5%).

The most common (≥20%) adverse reactions, including laboratory abnormalities, were decreased neutrophils, decreased hemoglobin, increased fasting glucose, decreased platelets, decreased lymphocytes, stomatitis, diarrhea, decreased calcium, fatigue, decreased potassium, increased creatinine, increased ALT, nausea, decreased sodium, decreased magnesium, rash, decreased appetite, COVID-19 infection, and headache.

Adverse reactions and laboratory abnormalities in INAVO120 are summarized in Table 3 and Table 4, respectively. Patient-reported symptoms are summarized in Table 5.

TABLE 3
Adverse Reactions (≥10% with ≥5% [All Grades] or ≥2% [Grade
3-4] Higher Incidence in the ITOVEBI Arm) in INAVO120

| | ITOVEBI + | | Placebo + | |
|---|---|---|---|---|
| | Palbociclib + | | Palbociclib + | |
| | Fulvestrant | | Fulvestrant | |
| | N = 162 | | N = 162 | |
| | All Grades | Grade 3-4 | All Grades | Grade 3-4 |
| Adverse Reaction | (%) | (%) | (%) | (%) |

Gastrointestinal Disorders

| | | | | |
|---|---|---|---|---|
| Stomatitis^{a} | 51 | 6* | 27 | 0 |
| Diarrhea | 48 | 3.7* | 16 | 0 |
| Nausea | 28 | 0.6* | 17 | 0 |
| Vomiting | 15 | 0.6* | 5 | 1.2* |

General Disorders and Administration Site Conditions

| | | | | |
|---|---|---|---|---|
| Fatigue | 38 | 1.9* | 25 | 1.2* |

Skin and Subcutaneous Tissue Disorders

| | | | | |
|---|---|---|---|---|
| Rash^{b} | 26 | 0 | 19 | 0 |
| Alopecia | 19 | 0 | 6 | 0 |
| Dry skin^{c} | 13 | 0 | 4.3 | 0 |

Metabolism and Nutrition Disorders

| | | | | |
|---|---|---|---|---|
| Decreased appetite | 24 | 0 | 9 | 0 |

Infections and Infestations

| | | | | |
|---|---|---|---|---|
| COVID-19 infection | 23 | 1.9 | 10 | 0.6 |
| Urinary tract infection^{b} | 15 | 1.2* | 9 | 0 |

Nervous System Disorders

| | | | | |
|---|---|---|---|---|
| Headache^{b} | 22 | 0 | 14 | 0 |

Investigations

| | | | | |
|---|---|---|---|---|
| Decreased weight | 17 | 3.7* | 0.6 | 0 |

*No Grade 4 adverse reactions were observed.
^{a}Includes aphthous ulcer, glossitis, glossodynia, lip ulceration, mouth ulceration, mucosal inflammation, and stomatitis.
^{b}Includes other related terms.
^{c}Includes dry skin, skin fissures, xerosis, and xeroderma.

Clinically relevant adverse reactions occurring in <10% of patients who received ITOVEBI in combination with palbociclib and fulvestrant included abdominal pain, dry eye, dysgeusia, and dyspepsia.

TABLE 4
Select Laboratory Abnormalities (≥10% with a ≥2% [All Grades or
Grade 3-4] Higher Incidence in the ITOVEBI Arm) in INAVO120

| | ITOVEBI + | | Placebo + | |
|---|---|---|---|---|
| | Palbociclib + | | Palbociclib + | |
| | Fulvestrant^{a} | | Fulvestrant^{b} | |
| Laboratory | All Grades | Grade 3-4 | All Grades | Grade 3-4 |
| Abnormality | (%) | (%) | (%) | (%) |

Hematology

| | | | | |
|---|---|---|---|---|
| Neutrophils (total, | 95 | 82 | 97 | 79 |
| absolute) decreased | | | | |
| Hemoglobin decreased | 88 | 8* | 85 | 2.5* |
| Platelets decreased | 84 | 16 | 71 | 3.7 |
| Lymphocytes (absolute) | 72 | 9 | 68 | 14 |
| decreased | | | | |

Chemistry

| | | | | |
|---|---|---|---|---|
| Glucose (fasting) | 85 | 12 | 43 | 0 |
| increased^{c} | | | | |
| Calcium decreased | 42 | 3.1 | 32 | 3.7 |
| Potassium decreased | 38 | 6 | 21 | 0.6* |
| Creatinine increased | 38 | 1.9* | 30 | 1.2* |
| ALT increased | 34 | 3.1* | 29 | 1.2* |
| Sodium decreased | 28 | 2.5* | 19 | 2.5 |
| Magnesium decreased | 27 | 0.6 | 21 | 0 |
| Lipase (fasting) | 16 | 1.4* | 7 | 0 |
| increased | | | | |

ALT = alanine aminotransferase
*No Grade 4 laboratory abnormalities were observed.
^{a}The denominator used to calculate the rate varied from 122 to 160 based on the number of patients with a baseline value and at least one post-treatment value.
^{b}The denominator used to calculate the rate varied from 131 to 161 based on the number of patients with a baseline value and at least one post-treatment value.
^{c}Grading according to CTCAE version 4.03.

In INAVO120, patient-reported symptomatic toxicities (i.e., diarrhea, nausea, vomiting, fatigue, mouth sores, decreased appetite, and rash) were assessed via the Patient-Reported Outcomes—Common Terminology Criteria for Adverse Events (PRO-CTCAE) at baseline, every two weeks through Cycle 3 Day 15, and then Day 1 of every other 28-day cycle until treatment discontinuation.

Completion rates in both arms were >90% at baseline and >80% at subsequent time points where >50% of randomized patients were on treatment.

TABLE 5
Patient-Reported Symptoms Assessed by PRO-CTCAE in INAVO120

| | Any Symptom Before | | Any Worsening on | | Worsening to | |
|---|---|---|---|---|---|---|
| | Treatment (%)^{b} | | Treatment (%)^{c} | | Score 3 or 4 (%)^{d} | |
| | ITOVEBI + | Placebo + | ITOVEBI + | Placebo + | ITOVEBI + | Placebo + |
| Symptom | P + F | P + F | P + F | P + F | P + F | P + F |
| (Attribute)^{a} | (N = 148)^{e} | (N = 152)^{e} | (N = 148)^{e} | (N = 152)^{e} | (N = 148)^{e} | (N = 152)^{e} |
| Diarrhea | 23 | 15 | 78 | 49 | 32 | 8 |
| (frequency), % | | | | | | |
| Nausea | 21 | 21 | 59 | 50 | 20 | 11 |
| (frequency), % | | | | | | |
| Vomiting | 9 | 6 | 35 | 26 | 6 | 3.3 |
| (frequency), % | | | | | | |
| Fatigue | 72 | 69 | 72 | 58 | 32 | 22 |
| (severity), % | | | | | | |
| Mouth sores | 11 | 14 | 74 | 52 | 30 | 9 |
| (severity), % | | | | | | |
| Decreased appetite | 38 | 27 | 78 | 55 | 26 | 12 |
| (severity), % | | | | | | |

| | |
|---|---|
| Baseline Presence | Post-baseline Presence |

| Symptom | ITOVEBI + P + F | Placebo + P + F | ITOVEBI + P + F | Placebo + P + F |
|---|---|---|---|---|
| (Attribute) | (N = 148) | (N = 152) | (N = 152) | (N = 158) |
| Rash (yes), % | 5 | 5 | 50 | 38 |

ITOVEBI + P + F = ITOVEBI with palbociclib and fulvestrant arm; Placebo + P + F = placebo with palbociclib and fulvestrant arm.
^{a}The symptom attribute scoring is defined by amount/frequency/severity with a score of 0 = ‘not at all’/‘never’/‘none’; 1 = ‘a little bit’/‘rarely’/‘mild’; 2 = ‘somewhat’/‘occasionally’/‘moderate’; 3 = ‘quite a bit’/‘frequently’/‘severe’; 4 = ‘very much’/‘almost constantly’/‘very severe’.
^{b}The percentage of patients whose symptom score before treatment was 1-4.
^{c}The percentage of patients whose symptom score increased during treatment, with respect to their score before treatment.
^{d}The percentage of patients whose symptom score increased to 3 or 4 during treatment, with respect to their score before treatment.
^{e}The number of patients who provided a score before treatment and at least one on-treatment score.

Patient-reported overall side-effect impact was assessed using the Modified Bother Item (MBI). Patients provided a response to “I am bothered by side effects of treatment,” and at baseline the proportion of patients with MBI responses of “not at all” were 70% in the ITOVEBI with palbociclib and fulvestrant arm and 76% in the placebo with palbociclib and fulvestrant arm. At Cycle 2 Day 15, the proportion of patients with MBI responses of “not at all” were 25% in the ITOVEBI with palbociclib and fulvestrant arm and 53% in the placebo with palbociclib and fulvestrant arm. Through 31 cycles of treatment, patients in the ITOVEBI with palbociclib and fulvestrant arm reported more side effect bother compared to the placebo with palbociclib and fulvestrant arm.

### 8 Use in Specific Populations

### 8.1 Pregnancy

### Risk Summary

ITOVEBI is used in combination with palbociclib and fulvestrant. Refer to the Full Prescribing Information of palbociclib and fulvestrant for pregnancy information.

Based on animal data and its mechanism of action, ITOVEBI can cause fetal harm when administered to a pregnant woman [see Clinical Pharmacology (12.1)]. There are no available data on the use of ITOVEBI in pregnant women to inform a drug-associated risk. In an animal reproduction study, oral administration of inavolisib to pregnant rats during the period of organogenesis caused adverse developmental outcomes, including embryo-fetal mortality, structural abnormalities, and alterations to growth at maternal exposures approximately equivalent to the human exposure at the recommended dose of 9 mg/day based on AUC (see Data). Advise pregnant women and females of reproductive potential of the potential risk to a fetus.

The background risk of major birth defects and miscarriage for the indicated population is unknown. In the U.S. general population, the estimated background risk of major birth defects is 2% to 4% and of miscarriage is 15% to 20% of clinically recognized pregnancies in the U.S. general population.

### Data

### Animal Data

In an embryo-fetal development study, pregnant rats received oral doses of inavolisib up to 6 mg/kg/day during the period of organogenesis. Administration of doses≥2 mg/kg/day resulted in decreases in fetal body weight and placental weight, post-implantation loss, lower fetal viability, fetal malformations (including kyphosis of vertebral column, fused thoracic arch, microphthalmia) and variations (including dilated renal pelvis, short supernumerary rib, wavy rib). At a dose of 2 mg/kg/day, maternal exposures were 0.9 times the human exposure at the recommended dose of 9 mg/day based on AUC.

### 8.2 Lactation

### Risk Summary

ITOVEBI is used in combination with palbociclib and fulvestrant. Refer to the Full Prescribing Information of palbociclib and fulvestrant for lactation information.

There are no data on the presence of inavolisib or its metabolites in human milk, its effects on milk production or a breastfed child. Because of the potential for serious adverse reactions in a breastfed child, advise lactating women to not breastfeed during treatment with ITOVEBI and for 1 week after the last dose.

### 8.3 Females and Males of Reproductive Potential

ITOVEBI is used in combination with palbociclib and fulvestrant. Refer to the Full Prescribing Information of palbociclib and fulvestrant for contraception and infertility information.

ITOVEBI can cause fetal harm when administered to a pregnant woman [see Use in Specific Populations (8.1)].

### Pregnancy Testing

Verify pregnancy status in females of reproductive potential prior to initiating treatment with ITOVEBI.

### Contraception

### Females

Advise females of reproductive potential to use effective non-hormonal contraception during treatment with ITOVEBI and for 1 week after the last dose.

### Males

Advise male patients with female partners of reproductive potential to use effective contraception during treatment with ITOVEBI and for 1 week after the last dose.

### Infertility

Based on animal studies, ITOVEBI may impair fertility in females and males of reproductive potential [see Nonclinical Toxicology (13.1)].

### 8.4 Pediatric Use

The safety and efficacy of ITOVEBI in pediatric patients have not been established.

### 8.5 Geriatric Use

Of the 162 patients who received ITOVEBI in INAVO120, 15% were ≥65 years of age, and 3% were ≥75 years of age.

Dosage modifications or interruptions of ITOVEBI due to adverse reactions occurred at a higher incidence for patients≥65 years of age compared to younger patients (79% versus 68%, respectively).

Clinical studies of ITOVEBI did not include sufficient numbers of patients≥65 years of age to determine whether they respond differently from younger patients.

### 8.6 Renal Impairment

Reduce the dosage in patients with moderate renal impairment (eGFR 30 to <60 mL/min based on CKD-EPI) [see Dosage and Administration (2.5)]. No dosage modification is recommended in patients with mild renal impairment (eGFR 60 to <90 mL/min). ITOVEBI has not been evaluated in patients with severe renal impairment (eGFR<30 mL/min).

### 11 Description

ITOVEBI contains inavolisib, a kinase inhibitor. The chemical name of inavolisib is (2S)-2-[[2-[(4S)-4-(difluoromethyl)-2-oxo-oxazolidin-3-yl]-5,6-dihydroimidazo[1,2-d][1,4]benzoxazepin-9-yl]amino]propanamide. Inavolisib is a white to off-white, greyish pink, greyish orange, or greyish yellow powder or powder with lumps. Inavolisib demonstrates pH-dependent aqueous solubility; the greatest solubility is at low pH, and solubility decreases with increasing pH. The molecular formula for inavolisib is C₁₈H₁₉F₂N₅O₄ and the molecular weight is 407.37 g/mol. The chemical structure of inavolisib is shown below:

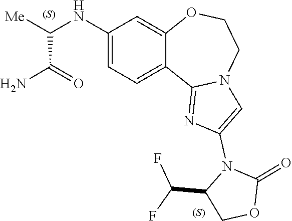

ITOVEBI film-coated tablets are supplied for oral administration with two strengths that contain 3 mg and 9 mg of inavolisib. The tablets also contain lactose, magnesium stearate, microcrystalline cellulose, and sodium starch glycolate. The film-coating contains the following inactive ingredients: polyvinyl alcohol (partially hydrolyzed), titanium dioxide, macrogol/polyethylene glycol, talc, iron oxide red, and iron oxide yellow (in the 9 mg tablet only).

### 12 Clinical Pharmacology

### 12.1 Mechanism of Action

Inavolisib is an inhibitor of phosphatidylinositol 3-kinase (PI3K) with inhibitory activity predominantly against PI3Kα. In vitro, inavolisib induced the degradation of mutated PI3K catalytic alpha subunit p110α (encoded by the PIK3CA gene), inhibited phosphorylation of the downstream target AKT, reduced cellular proliferation, and induced apoptosis in PIK3CA-mutated breast cancer cell lines. In vivo, inavolisib reduced tumor growth in PIK3CA-mutated, estrogen receptor-positive, breast cancer xenograft models. The combination of inavolisib with palbociclib and fulvestrant increased tumor growth inhibition compared to each treatment alone or the doublet combinations.

### 12.2 Pharmacodynamics

### Exposure-Response Relationships

The exposure-response relationship for the efficacy of inavolisib has not been fully characterized. Inavolisib time course of pharmacodynamic response is unknown. Higher systemic exposure of inavolisib was associated with higher incidence of Grade≥2 anemia, Grade≥2 hyperglycemia, and inavolisib dosage modifications due to adverse reactions.

### Cardiac Electrophysiology

At the recommended approved dosage, a mean increase in the QTc interval of ≥20 ms is unlikely.

### 12.3 Pharmacokinetics

Inavolisib pharmacokinetics are presented as geometric mean (geometric coefficient of variation [geo CV]%) following administration of the approved recommended dosage unless otherwise specified. The inavolisib steady-state AUC is 1010 h*ng/mL (25%) and Cₘₐₓ is 69 ng/mL (27%). Steady-state concentrations are predicted to be attained by day 5.

Inavolisib accumulation is approximately 2-fold.

Inavolisib steady-state AUC is proportional with dose from 6 to 12 mg (0.7 to 1.3 times the approved recommended dosage).

### Absorption

Inavolisib absolute oral bioavailability is 76%. Inavolisib steady-state median (min, max) time to maximum plasma concentration (Tₘₐₓ) is 3 hours (0.5, 4 hours).

### Effect of Food

No clinically significant differences in inavolisib pharmacokinetics were observed following administration of inavolisib with a high-fat meal (approximately 1,000 calories, 50% fat).

### Distribution

Inavolisib apparent (oral) volume of distribution is 155 L (26%). Inavolisib plasma protein binding is 37% and is not concentration-dependent in vitro. Inavolisib blood-to-plasma ratio is 0.8.

### Elimination

Inavolisib elimination half-life is 15 hours (24%) with a total clearance of 8.8 L/hr (29%).

### Metabolism

Inavolisib is primarily metabolized by hydrolysis. In vitro, inavolisib was minimally metabolized by CYP3A.

### Excretion

Following oral administration of a single radiolabeled dose, 49% of the administered dose was recovered in urine (40% unchanged) and 48% in feces (11% unchanged).

### Specific Populations

No clinically significant differences in the pharmacokinetics of inavolisib were observed based on age (27 to 85 years), sex, race (Asian or White), body weight (39 to 159 kg), or mild hepatic impairment (total bilirubin>ULN to ≤1.5′ ULN or AST>ULN and total bilirubin≤ULN). The effect of moderate to severe hepatic impairment on inavolisib pharmacokinetics is unknown.

Patients with Renal Impairment

Inavolisib AUC was 73% higher in patients with moderate renal impairment compared to patients with normal renal function (eGFR≥90 mL/min).

No clinically significant differences in the pharmacokinetics of inavolisib were observed in patients with mild renal impairment compared to patients with normal renal function. The effect of severe renal impairment (eGFR 30 mL/min) on the pharmacokinetics of inavolisib is unknown.

### Drug Interaction Studies

### Clinical Studies and Model-Informed Approaches

Proton Pump Inhibitors: No clinically significant difference in steady-state inavolisib pharmacokinetics were observed based upon concomitant use of a proton pump inhibitor (lansoprazole, omeprazole, esomeprazole, pantoprazole, or rabeprazole).

### In Vitro Studies

CYP450 Enzymes: Inavolisib induces CYP3A and CYP2B6. Inavolisib is a time-dependent inhibitor of CYP3A. Inavolisib does not inhibit CYP1A2, CYP2B6, CYP2C8, CYP2C9, CYP2C19, or CYP2D6.

Transporter Systems: Inavolisib is a substrate of P-glycoprotein (P-gp) and breast cancer resistance protein (BCRP), but is not a substrate of OATP1B1, OATP1B3, OCT1, OCT2, MATE1, MATE2K, OAT1, OAT2. Inavolisib does not inhibit P-gp, BCRP, OATP1B1, OATP1B3, OCT1, OCT2, OAT1, OAT3, MATE1, or MATE2K.

### 13 Nonclinical Toxicology

### 13.1 Carcinogenesis, Mutagenesis, Impairment of Fertility

Carcinogenicity studies with inavolisib have not been conducted.

Inavolisib was not mutagenic in the bacterial reverse mutation (Ames) assay. Inavolisib was clastogenic in an in vitro human lymphocyte micronucleus assay. Inavolisib was not genotoxic in an in vivo rat bone marrow micronucleus test and did not induce DNA break in a liver comet assay.

Fertility studies with inavolisib have not been conducted. In repeat-dose toxicity studies, inavolisib was administered orally once daily for up to 3 months duration in rats and dogs.

In male rats, dose-dependent atrophy of the prostate and seminal vesicle and decreased organ weights of the prostate, seminal vesicle, epididymis and testis were observed at doses≥1.5 mg/kg/day (≥0.4 times the human exposure at the recommended dose of 9 mg/day based on AUC). In male dogs, focal inspissation of seminiferous tubule contents and multinucleated spermatids in the testis and epithelial degeneration/necrosis in the epididymis were observed following 4 weeks of dosing at ≥1.5 mg/kg/day (≥2 times the human exposure at the recommended dose of 9 mg/day based on AUC) and decreased sperm count was observed following 3 months of dosing at ≥1 mg/kg/day (≥1.2 times the human exposure at the recommended dose of 9 mg/day based on AUC). Findings in dogs were not observed following a recovery period.

In female rats, atrophy in the uterus and vagina, decreased ovarian follicles, and findings suggestive of an interruption/alteration of the estrous cycle were observed following up to 3 months of dosing at doses≥3 mg/kg/day (≥1.2 times the human exposure at the recommended dose of 9 mg/day based on AUC). Findings in the uterus, vagina, and estrous cycle observed in the 4-week toxicity study were not observed following recovery. Recovery was not assessed in the 3-month study in rats.

13.2 Animal Toxicology and/or Pharmacology

Lens degeneration, characterized by lens fiber swelling, separation of lens fibers, and/or accumulation of subcapsular proteinaceous material, was observed in rats at an oral inavolisib dose of 10 mg/kg/day (6.3 times the human exposure at the recommended dose of 9 mg/day based on AUC). In dogs, lens fiber swelling and lens cortex vacuolation were observed at oral inavolisib doses≥0.3 mg/kg/day (≥0.5 times the human exposure at the recommended dose based on AUC) and ≥1 mg/kg/day (≥1.2 times the human exposure at the recommended dose based on AUC), respectively. Lens degeneration was present in rats following a 4-week recovery period but was not present in dogs following a 12-week recovery period.

### 14 Clinical Studies

### 14.1 Locally Advanced or Metastatic Breast Cancer

### INAVO120

INAVO120 (NCT04191499) was a randomized (1:1), double-blind, placebo-controlled trial evaluating the efficacy of ITOVEBI in combination with palbociclib and fulvestrant in adult patients with endocrine-resistant PIK3CA-mutated, HR-positive, HER2-negative (defined as IHC 0 or 1+, or IHC 2+/ISH−), locally advanced or metastatic breast cancer whose disease progressed during or within 12 months of completing adjuvant endocrine therapy and who have not received prior systemic therapy for locally advanced or metastatic disease. Randomization was stratified by presence of visceral disease (yes or no), endocrine resistance (primary or secondary), and geographic region (North America/Western Europe, Asia, other).

Primary endocrine resistance was defined as relapse while on the first 2 years of adjuvant endocrine therapy (ET) and secondary endocrine resistance was defined as relapse while on adjuvant ET after at least 2 years or relapse within 12 months of completing adjuvant ET.

Patients were required to have HbA_{1C}<6% and fasting blood glucose<126 mg/dL. The study excluded patients with Type 1 diabetes mellitus or Type 2 diabetes mellitus requiring ongoing anti-hyperglycemic treatment at the start of study treatment.

PIK3CA mutation status was prospectively determined in a central laboratory using the FoundationOne® Liquid CDx assay on plasma-derived circulating tumor DNA (ctDNA) or in local laboratories using various validated polymerase chain reaction (PCR) or next-generation sequencing (NGS) assays on tumor tissue or plasma. All patients were required to provide both a freshly collected pre-treatment blood sample and a tumor tissue sample for central evaluation and determination of PIK3CA mutation(s) status.

Patients received either ITOVEBI 9 mg (n=161) or placebo (n=164) orally once daily, in combination with palbociclib 125 mg orally once daily for 21 consecutive days followed by 7 days off treatment to comprise a cycle of 28 days, and fulvestrant 500 mg administered intramuscularly on Cycle 1, Days 1 and 15, and then on Day 1 of every 28-day cycle. Patients received treatment until disease progression or unacceptable toxicity. In addition, all pre/perimenopausal women and men received an LHRH agonist throughout therapy.

The baseline demographic and disease characteristics were: median age 54 years (range: 27 to 79 years); 98% female, of which 39% were pre/perimenopausal; 59% White, 38% Asian, 2.5% unknown, 0.6% Black or African American; 6% Hispanic or Latino; and Eastern Cooperative Oncology Group (ECOG) performance status of 0 (63%) or 1 (36%). Tamoxifen (57%) and aromatase inhibitors (50%) were the most commonly used adjuvant endocrine therapies. Sixty-four percent of patients were considered to have secondary endocrine resistance. Eighty-three percent of patients had received prior chemotherapy (in the neo/adjuvant setting) and 1.2% of patients had been treated with a CDK4/6 inhibitor.

The major efficacy outcome measure was investigator (INV)-assessed progression-free survival (PFS) per Response Evaluation Criteria in Solid Tumors (RECIST) version 1.1. Additional efficacy outcome measures included overall survival (OS), INV-assessed objective response rate (ORR), and INV-assessed duration of response (DOR).

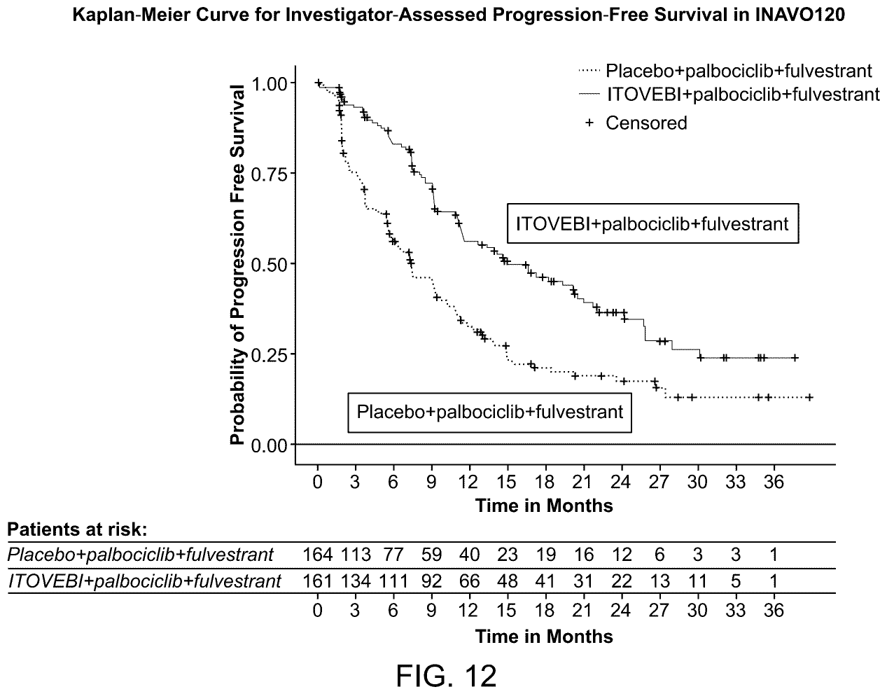

Efficacy results are summarized in Table 6 and FIG. 12. INV-assessed PFS results were supported by consistent results from a blinded independent central review (BICR) assessment. At the time of the PFS analysis, OS data were not mature with 30% deaths in the overall population.

TABLE 6
Efficacy Results in Patients with Locally Advanced
or Metastatic Breast Cancer in INAVO120

| | | |
|---|---|---|
| | ITOVEBI + | Placebo + |
| | Palbociclib + | Palbociclib + |
| | Fulvestrant | Fulvestrant |
| Efficacy Endpoint | N = 161 | N = 164 |

Progression-Free Survival^{a, b}

| | | | | |
|---|---|---|---|---|
| Patients with | 82 | (51) | 113 | (69) |
| event, n (%) | | | | |
| Median, months | 15.0 | (11.3, 20.5) | 7.3 | (5.6, 9.3) |
| (95% CI) | | | | |

| | |
|---|---|
| Hazard ratio | 0.43 (0.32, 0.59) |
| (95% CI) | |
| p-value | <0.0001 |

Objective Response Rate^{a, b, c}

| | | | | |
|---|---|---|---|---|
| Patients with | 94 | (58) | 41 | (25) |
| CR or PR, n (%) | | | | |

| | | |
|---|---|---|
| 95% CI | (50, 66) | (19, 32) |

Duration of Response^{b}

| | | | | |
|---|---|---|---|---|
| Median DOR, | 18.4 | (10.4, 22.2) | 9.6 | (7.4, 16.6) |
| months (95% CI) | | | | |

CI = confidence interval;
CR = complete response;
DOR = duration of response;
PR = partial response
^{a}Per RECIST version 1.1.
^{b}Based on investigator assessment.
^{c}Based on confirmed ORR.

### 16 how Supplied/Storage and Handling

ITOVEBI is supplied in the following strengths and package configurations:

| Package | Tablet | | |
|---|---|---|---|
| Configuration | Strength | NDC | Tablet Description |
| Bottle of | 3 mg | 50242-084-01 | Red and round convex- |
| 28 tablets | | | shaped with an “INA 3” |
| | | | debossing on one side |
| Bottle of | 9 mg | 50242-079-01 | Pink and oval-shaped with |
| 28 tablets | | | an “INA 9” debossing on |
| | | | one side |

Store at 20° C. to 25° C. (68° F. to 77° F.), excursions permitted between 15° C. and 30° C. (between 59° F. and 86° F.) [see USP Controlled Room Temperature].

### 17 Patient Counseling Information

Advise the patient to read the FDA-approved patient labeling (Patient Information).

### Hyperglycemia

Advise patients that ITOVEBI may cause hyperglycemia and the need to monitor fasting glucose levels periodically during therapy. Advise patients to contact their healthcare provider immediately for signs and symptoms of hyperglycemia (e.g., excessive thirst, urinating more often, blurred vision, mental confusion, difficulty breathing, or increased appetite with weight loss) [see Warnings and Precautions (5.1)].

### Stomatitis

Advise patients that ITOVEBI may cause stomatitis, which may be severe, and to notify their healthcare provider if they develop symptoms of stomatitis (e.g., painful redness, swelling, or sores in the mouth) [see Warnings and Precautions (5.2)].

### Diarrhea

Advise patients that ITOVEBI may cause diarrhea, which may be severe, and to start antidiarrheal treatment, increase oral fluids, and notify their healthcare provider if diarrhea occurs while taking ITOVEBI [see Warnings and Precautions (5.3)].

### Embryo-Fetal Toxicity

Inform pregnant women and females of reproductive potential of the potential risk to a fetus. Advise females to inform their healthcare provider of a known or suspected pregnancy [see Warnings and Precautions (5.4) and Use in Specific Populations (8.1)].

Advise females of reproductive potential to use effective non-hormonal contraception during treatment with ITOVEBI and for 1 week after the last dose [see Use in Specific Populations (8.3)].

Advise male patients with female partners of reproductive potential to use effective contraception during treatment with ITOVEBI and for 1 week after the last dose [see Use in Specific Populations (8.3)].

Refer to the Full Prescribing Information of palbociclib and fulvestrant for pregnancy and contraception information.

### Lactation

Advise women not to breastfeed during treatment with ITOVEBI and for 1 week after the last dose [see Use in Specific Populations (8.2)]. Refer to the Full Prescribing Information of palbociclib and fulvestrant for lactation information.

### Infertility

Advise females and males of reproductive potential that ITOVEBI may impair fertility [see Use in Specific Populations (8.3)]. Refer to the Full Prescribing Information of palbociclib and fulvestrant for infertility information.

### Dosing

Instruct patients to take ITOVEBI at approximately the same time each day [see Dosage and Administration (2.3)].

Instruct patients that if a dose of ITOVEBI is missed, to take the missed dose as soon as possible within 9 hours. After more than 9 hours, skip the dose and take the next dose at the scheduled time [see Dosage and Administration (2.3)].

Instruct patients that if vomiting occurs after taking the ITOVEBI dose, do not take an additional dose on that day. Resume the usual dosing schedule the next day [see Dosage and Administration (2.3)].

### Example 4 Inavolisib-Based Therapy in PIK3CA-Mutated Advanced Breast Cancer

Inavolisib is a highly potent and selective PI3Kα inhibitor that facilitates degradation of mutated p110α. Inavolisib with palbociclib and fulvestrant has shown synergistic activity in preclinical models and promising antitumor activity in early-phase trials.

In a phase 3, randomized, double-blind trial, we compared first-line inavolisib (9 mg once daily) plus palbociclib plus fulvestrant with placebo plus palbociclib plus fulvestrant in 325 patients with PIK3CA-mutated, hormone receptor-positive, HER2-negative locally advanced/metastatic breast cancer (HR+, HER2− LA/mBC), who relapsed during or within 12 months of adjuvant endocrine therapy completion. The primary end point was investigator-assessed progression-free survival.

At a median follow-up of 21.3 months, median progression-free survival was 15.0 months (95% confidence interval [CI], 11.3 to 20.5) in the inavolisib group and 7.3 months (5.6 to 9.3) in the placebo group (hazard ratio 0.43; 95% CI, 0.32 to 0.59; P<0.001). Objective response rates were 58.4% in the inavolisib group and 25.0% in the placebo group. Rates of selected Grade 3-4 adverse events in the inavolisib versus placebo groups were: neutropenia, 80.2% versus 78.4%; hyperglycemia, 5.6% versus 0%; stomatitis and mucosal inflammation, 5.6% versus 0%; diarrhea, 3.7% versus 0%. No grade 3-4 rash was observed. Rates of discontinuation of any study treatment due to adverse events were 6.8% and 0.6% in the inavolisib and placebo groups, respectively.

Inavolisib plus palbociclib and fulvestrant significantly improved progression-free survival in patients with PIK3CA-mutated, HR+, HER2− LA/mBC, with low rates of discontinuation related to adverse events.

### Introduction

Activating PIK3CA mutations are frequent (approximately 35-40%) in hormone receptor-positive breast cancer (HR+BC),¹⁻⁴ are a poor prognostic factor in advanced breast cancer, and a predictive biomarker of response to phosphatidylinositol-4,5-bisphosphate 3-kinase (PI3K) inhibitors.^{1,35-7} Clinical trials have established regimens that target the key oncogenic drivers of HR+BC, including combinations of endocrine therapy with cyclin-dependent kinase 4/6 (CDK4/6) inhibitors,⁸⁻¹⁰ or with PI3K/protein kinase B (AKT)/mammalian target of rapamycin (mTOR) inhibitors; however, treatment resistance remains a major challenge in clinical practice.¹¹⁻¹³

The three key oncogenic pathways (estrogen receptor [ER], CDK4/6, PI3K) that drive HR+, locally advanced/metastatic BC (LA/mBC) are highly interconnected, with complex feedback mechanisms that may drive adaptation/resistance.¹⁴ An effective treatment regimen with an acceptable level of safety that enables co-targeting of all three signaling pathways is needed. Preclinical research showed that substantial synergy can be achieved with simultaneous blockade of the ER, CDK4/6, and PI3K pathways in PIK3CA-mutated xenograft models by deepening responses and preventing/delaying resistance.¹⁵⁻¹⁷ However, previous attempts to exploit this approach in the clinic have been unsuccessful, largely in part due to treatment side effects. 1-20

Inavolisib is a highly potent and selective inhibitor of the p110 catalytic subunit alpha isoform (p110α; encoded by PIK3CA) of the PI3K complex that also promotes the degradation of mutated p110α. Previous PI3K inhibitors have been toxic and poorly tolerated when combined with standard-of-care agents;^{11,21} the enhanced selectivity for p 110a and degradation of mutated p110α with inavolisib may lead to a wider therapeutic window, enabling drug combinations and sustained pathway inhibition.^{17,22,23} A first-in-human phase 1 study (NCT03006172) demonstrated the ability of inavolisib to be combined with palbociclib plus fulvestrant at the maximum single-agent dose for each drug, with no drug-drug interactions, acceptable tolerability, and promising preliminary antitumor activity in PIK3CA-mutated, HR+, HER2− negative (HER2−) LA/mBC.²⁴ Preclinical and clinical data have demonstrated that tumors harboring a broad range of PIK3CA hotspot mutations occurring at major functional domains (i.e., helical, kinase, C2, etc.) are sensitive to inavolisib alone or in combination with standard therapies for patients with PIK3CA-mutated, HR+, HER2− breast cancer.^{17,25,26}

INAVO120 (NCT04191499) is a phase 3, randomized, double-blind, placebo-controlled study that assessed first-line inavolisib or placebo with palbociclib plus fulvestrant in patients with PIK3CA-mutated, HR+, HER2− LA/mBC with disease recurrence on or within 12 months of completing adjuvant endocrine therapy; the trial was enriched for patients with clinicopathologic characteristics associated with poor prognosis.

### Methods

### Patients

The study enrolled pre-, peri-, and postmenopausal women, and men, who had PIK3CA-mutated, HR+, HER2− LA/mBC, disease recurrence/progression during or within 12 months of adjuvant endocrine therapy completion (patients with de novo metastatic breast cancer were excluded), fasting glucose<126 mg/dL, HbA_{1C}<6.0%, and measurable disease by Response Evaluation Criteria in Solid Tumors (RECIST) v1.1.²⁷ PIK3CA-mutation positivity was determined by local testing of tumor tissue or circulating tumor DNA (ctDNA) with an appropriately validated polymerase chain reaction or next-generation sequencing (NGS) test performed at a Clinical Laboratory Improvement Amendments- or equivalently-certified laboratory, or by testing of ctDNA at a Sponsor-designated central laboratory. Central ctDNA testing was primarily done using the FoundationOne® Liquid CDx (F1LCDx) NGS assay (Foundation Medicine, Inc., Cambridge, MA, USA); in China, the central ctDNA test was the PredicineCARE NGS assay (Huidu, Shanghai, China).

### Trial Design and Treatment

Patients were randomized 1:1 using a permuted-block method to inavolisib (9 mg orally once daily [PO QD] on Days 1-28 of each 28-day cycle) or placebo (PO QD) given with palbociclib (125 mg PO QD on Days 1-21 of each 28-day cycle) plus fulvestrant (500 mg intramuscularly on Cycle 1 Days 1 and 15, and approximately every 28 days thereafter). Pre- and perimenopausal women, and men, received hormonal suppression via luteinizing hormone-releasing hormone agonists for the duration of the trial treatment period. Treatment continued until disease progression/unacceptable toxicity/withdrawal of consent/death. Dose modifications for inavolisib and palbociclib are described in the protocol; dose reductions for fulvestrant were not allowed. Patients who discontinued any study drug due to intolerance were able to continue receiving other study drugs.

Randomization stratification factors were visceral disease (yes vs. no), endocrine resistance (primary vs. secondary), and region (North America/Western Europe vs. Asia vs. Other). Primary endocrine resistance was defined as relapse while on the first 2 years of adjuvant endocrine therapy; secondary, as relapse while on adjuvant endocrine therapy after at least 2 years/relapse within 12 months of completing adjuvant endocrine therapy.²⁸

### End Points

The primary end point was investigator-assessed progression-free survival (time from randomization to the first occurrence of disease progression per RECIST v1.1 or death from any cause, whichever occurs first). Patients without disease progression or death were censored at the time of the last tumor assessment (or at the time of randomization if no tumor assessment was performed after the baseline visit). Secondary end points included overall survival; investigator-assessed confirmed objective response rate, best overall response, clinical benefit rate, and response duration; and patient-reported outcomes. Safety, tolerability, and pharmacokinetics were also assessed.

Severity of adverse events was determined per National Cancer Institute Common Terminology Criteria for Adverse Events version 5.0.²⁹ Selected adverse events were defined based on the known safety profile of inavolisib and protocol-defined adverse events of special interest and were assessed using grouped terms.

### Statistical Analysis

INAVO120 was designed to detect a hazard ratio of 0.65 for a progression-free survival comparison of inavolisib versus placebo using a two-sided log-rank test at the 0.05 level of significance, corresponding to an increase in median progression-free survival of 5.9 months. The primary analysis was planned after ˜194 events (85% power). Hazard ratios and corresponding 95% confidence intervals (CIs) were estimated using a stratified Cox proportional hazards model. The Kaplan-Meier approach was used to estimate median progression-free survival for each group, and the Brookmeyer-Crowley method was used to construct the corresponding 95% CIs. The subgroup analysis for progression-free survival based on baseline characteristics was prespecified. Overall survival was to be hierarchically tested if the progression-free survival difference was statistically significant. An interim analysis was planned at the primary progression-free survival analysis (prespecified boundary, P<0.0098), and the final analysis is planned after ˜153 deaths.

Efficacy end point analyses were performed in the Full Analysis Set (all randomized patients, whether or not the assigned study treatment was received). Response duration was assessed in all randomized patients who had an objective response. Safety analyses were conducted in all patients who received ≥1 dose of any study drug (Safety Analysis Set); participants were analyzed according to treatment received.

Secondary efficacy end point analyses were performed with Type I error control.

### Results

### Patients

Between 29 Jan. 2020-14 Sep. 2023, 325 patients were enrolled. Most patients' tumor PIK3CA mutation status was determined through ctDNA-based testing. Median follow-up was 21.3 months and 21.5 months in the inavolisib and placebo groups, respectively (data cutoff: 29 Sep. 2023).

Baseline characteristics were well balanced between groups (Table B1); median age was 54.0 years; 60.0% of patients were postmenopausal. Disease burden was high overall: 51.4% had metastases in ≥3 organs; 80.0%, visceral disease; 51.7%, liver metastases. Most patients had prior (neo)adjuvant chemotherapy (82.8%), had not received a prior CDK4/6 inhibitor (98.8%), and 47.7% had prior (neo)adjuvant tamoxifen only. The distribution of relevant risk factors was well balanced but Black or African American patients were underrepresented.

TABLE B1
Patient Demographics and Baseline Characteristics (Full Analysis Set)

| | Inavolisib + | | Placebo + | | | |
|---|---|---|---|---|---|---|
| | palbociclib + | | palbociclib + | | All | |
| | fulvestrant | | fulvestrant | | patients | |
| Characteristic | (n = 161) | | (n = 164) | | (N = 325) | |
| Median age (range) - years | 53.0 | (27-77) | 54.5 | (29-79) | 54.0 | (27-79) |
| Female sex - no. (%) | 156 | (96.9) | 163 | (99.4) | 319 | (98.2) |
| Race - no. (%) | | | | | | |
| Asian | 61 | (37.9) | 63 | (38.4) | 124 | (38.2) |
| Black or African American | 1 | (0.6) | 1 | (0.6) | 2 | (0.6) |
| White | 94 | (58.4) | 97 | (59.1) | 191 | (58.8) |
| ECOG PS - no. (%) | | | | | | |
| 0 | 100 | (62.1) | 106 | (64.6) | 206 | (63.4) |
| 1 | 60 | (37.3) | 58 | (35.4) | 118 | (36.3) |
| Menopausal status at | | | | | | |
| randomization - no. (%) | | | | | | |
| Premenopausal | 65 | (40.4) | 59 | (36.0) | 124 | (38.2) |
| Postmenopausal | 91 | (56.5) | 104 | (63.4) | 195 | (60.0) |
| Median weight (range), kg | 62.5 | (39-124) | 64.0 | (38-111) | 63.0 | (38-124) |
| Body mass index - no. (%) | | | | | | |
| <18.5 kg/m² | 8 | (5.0) | 10 | (6.1) | 18 | (5.5) |
| ≥18.5 kg/m² to <25.0 kg/m² | 78 | (48.4) | 75 | (45.7) | 153 | (47.1) |
| ≥25.0 kg/m² to <0.35 kg/m² | 44 | (27.3) | 50 | (30.5) | 94 | (28.9) |
| ≥30 kg/m² | 29 | (18.0) | 28 | (17.1) | 57 | (17.5) |
| Missing | 2 | (1.2) | 1 | (0.6) | 3 | (0.9) |
| Number of organ sites - | | | | | | |
| no. (%) | | | | | | |
| 1 | 21 | (13.0) | 32 | (19.5) | 53 | (16.3) |
| 2 | 59 | (36.6) | 46 | (28.0) | 105 | (32.3) |
| ≥3 | 81 | (50.3) | 86 | (52.4) | 167 | (51.4) |
| Visceral disease - no. (%)* | 132 | (82.0) | 128 | (78.0) | 260 | (80.0) |
| Liver | 77 | (47.8) | 91 | (55.5) | 168 | (51.7) |
| Lung | 66 | (41.0) | 66 | (40.2) | 132 | (40.6) |
| Bone only^{†} | 5 | (3.1) | 6 | (3.7) | 11 | (3.4) |
| ER^{‡} and PgR status - no. (%) | | | | | | |
| ER-positive/PgR-positive | 113 | (70.2) | 113 | (68.9) | 226 | (69.5) |
| ER-positive/PgR-negative | 45 | (28.0) | 45 | (27.4) | 90 | (27.7) |
| Endocrine resistance - | | | | | | |
| no. (%) | | | | | | |
| Primary | 53 | (32.9) | 58 | (35.4) | 111 | (34.2) |
| Secondary | 108 | (67.1) | 105 | (64.0) | 213 | (65.5) |
| Prior (neo)adjuvant | 132 | (82.0) | 137 | (83.5) | 269 | (82.8) |
| chemotherapy - no. (%) | | | | | | |
| Prior (neo)adjuvant | 160 | (99.4) | 163 | (99.4) | 323 | (99.4) |
| endocrine therapy - no. (%) | | | | | | |
| Aromatase inhibitor only | 60 | (37.3) | 71 | (43.3) | 131 | (40.3) |
| Tamoxifen only | 82 | (50.9) | 73 | (44.5) | 155 | (47.7) |
| Aromatase inhibitor and | 18 | (11.2) | 19 | (11.6) | 37 | (11.4) |
| tamoxifen | | | | | | |
| Prior (neo)adjuvant CDK4/6 | 3 | (1.9) | 1 | (0.6) | 4 | (1.2) |
| inhibitor - no. (%) | | | | | | |

*“Visceral” (yes/no) refers to lung, liver, brain, pleural, and peritoneal involvement.
^{†}Patients with evaluable bone-only disease were not eligible; patients with bone-only and at least one measurable soft tissue component per RECIST 1.1, were eligible.
^{‡}Defined as ≥1% per ASCO CAP guidelines.
^{§} Other: three patients (1.9%) in the inavolisib arm and six patients (3.7%) in the placebo arm.
^{∥} Endocrine resistance was defined per the 4^{th} ESO-ESMO International Consensus Guidelines for Advanced Breast Cancer. Primary resistance: relapse while on the first 2 years of adjuvant endocrine therapy. Secondary resistance: relapse while on adjuvant endocrine therapy after at least 2 years or relapse within 12 months of completing adjuvant endocrine therapy.
ASCO denotes American Society of Clinical Oncology, CAP College of American Pathologists, ECOG PS Eastern Cooperative Oncology Group Performance Status, ER estrogen receptor, ESMO, European Society of Medical Oncology, ESO European School of Oncology, PgR progesterone receptor, RECIST Response Evaluation Criteria in Solid Tumors.

### Treatment

In the Safety Analysis Set, patients in the inavolisib group received a median of 9.2 months of inavolisib, 9.1 months of palbociclib, and 8.6 months of fulvestrant; median relative dose intensities were 95.8%, 87.3%, and 100.0%, respectively. Patients in the placebo group received a median of 5.6 months of placebo, 5.6 months of palbociclib, and 5.6 months of fulvestrant; the median relative dose intensities were 88.4% for palbociclib and 100.0% for fulvestrant.

### Efficacy

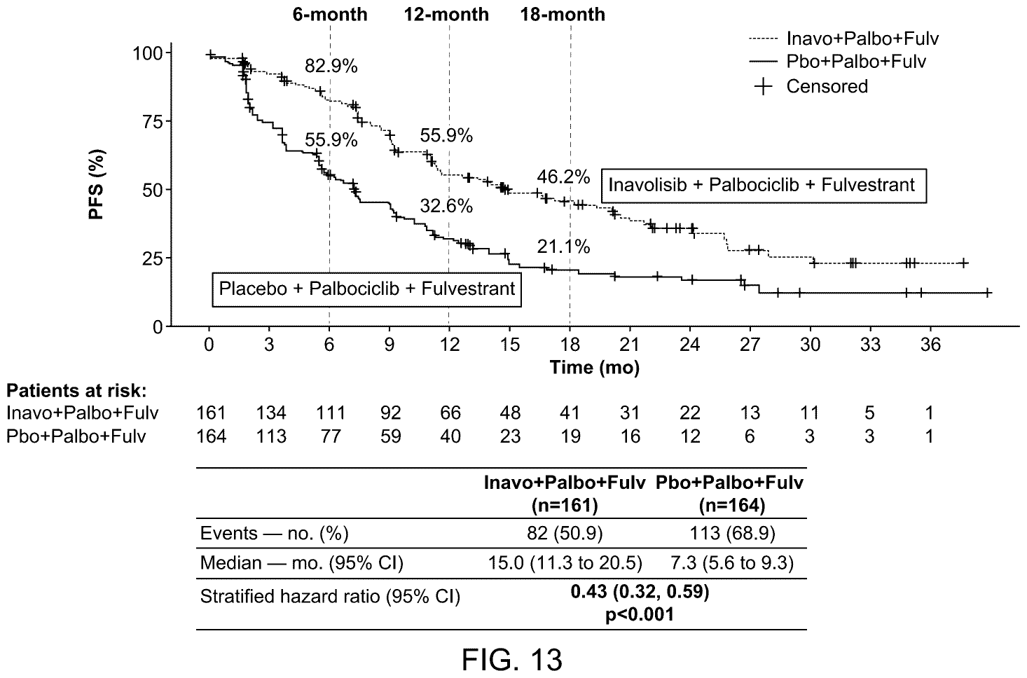

In the Full Analysis Set (n=161 and n=164 in the inavolisib and placebo groups, respectively), the median progression-free survival was 15.0 months versus 7.3 months in the inavolisib and placebo groups (stratified hazard ratio 0.43; 95% CI, 0.32 to 0.59; P<0.001), respectively (FIG. 13). Landmark progression-free survival event-free rates at 6, 12, and 18 months were 82.9%, 55.9%, and 46.2% in the inavolisib group, and 55.9%, 32.6%, and 21.1% in the placebo group. Subgroup analysis of progression-free survival revealed a generally consistent effect across key subgroups, including patients with and without visceral disease and liver metastases, though numbers of participants in certain subsets are small (FIG. 14). Progression-free survival was also assessed by blinded independent central review as a sensitivity analysis; results were consistent with investigator-assessed progression-free survival (stratified hazard ratio, 0.50; 95% CI, 0.36 to 0.68; P<0.001).

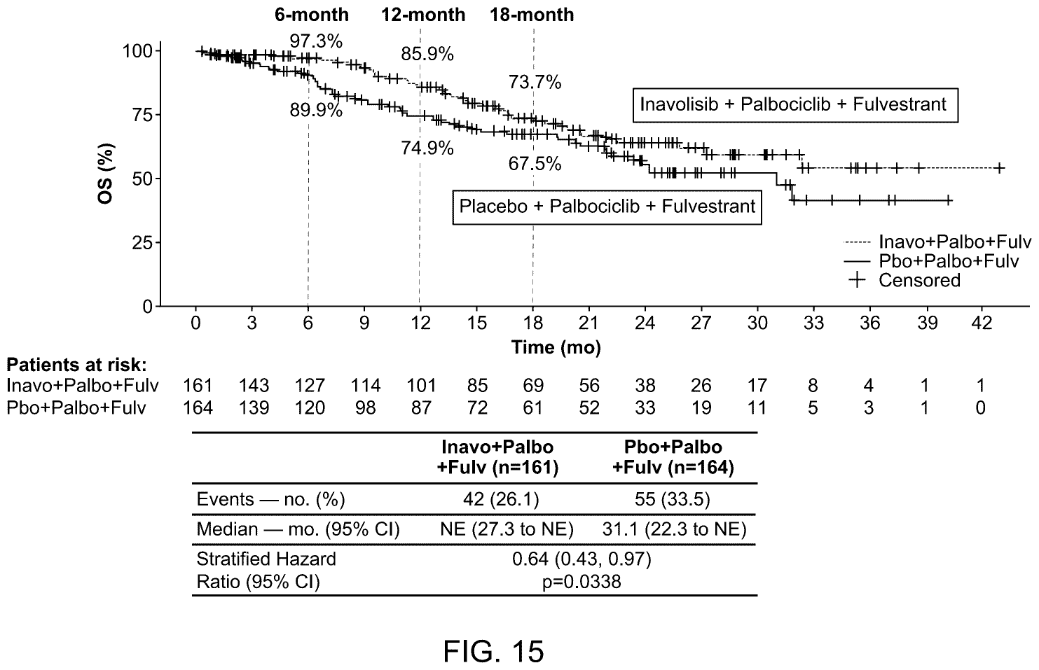

At the interim overall survival analysis, landmark overall survival event-free rates at 6, 12, and 18 months were 97.3%, 85.9%, and 73.7% in the inavolisib group, and 89.9%, 74.9%, and 67.5% in the placebo group. The stratified hazard ratio was 0.64 (95% CI, 0.43 to 0.97; P=0.0338, which did not cross the predefined significance boundary [P<0.0098]) (FIG. 15).

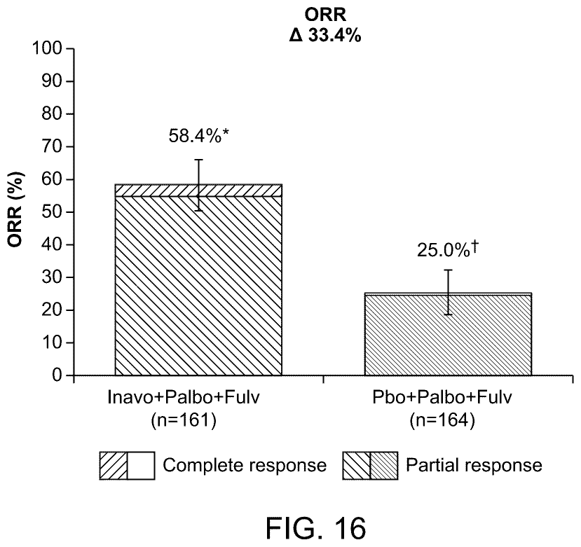

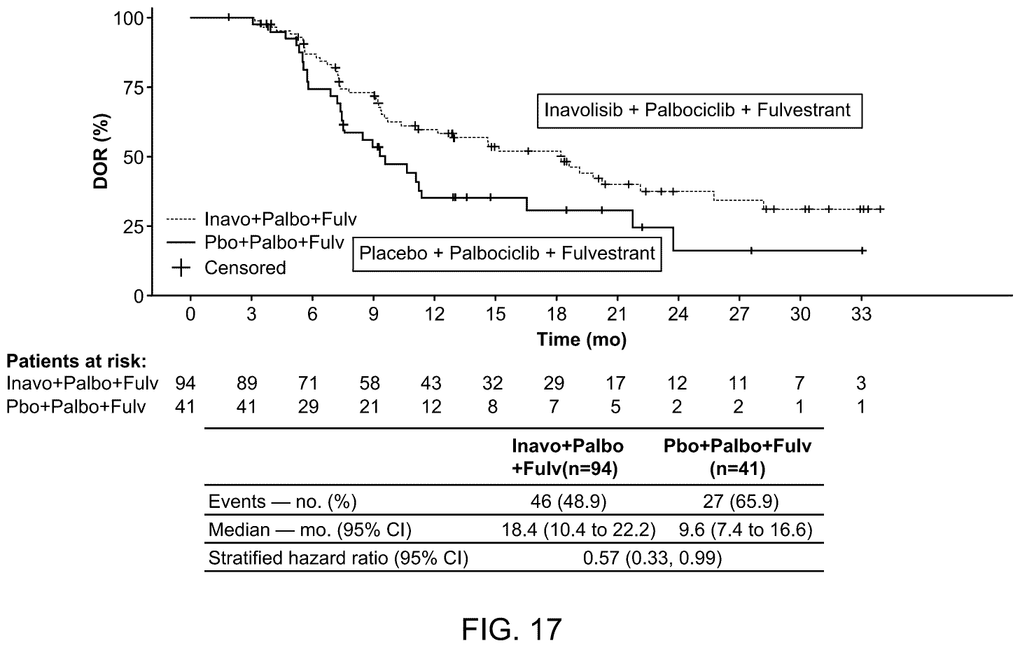

Response rate were 58.4% and 25.0% in the inavolisib and placebo groups (A33.4%; 95% CI, 23.3 to 43.5), respectively; median response duration was 18.4 months and 9.6 months, respectively (hazard ratio 0.57; 95% CI, 0.33 to 0.99) (FIG. 16 and FIG. 17).

### Safety

In the Safety Analysis Set (n=162 per group), the number of patients with ≥1 adverse event was 98.8% and 100% in the inavolisib and placebo groups, respectively. Selected adverse events occurring at any grade in ≥20% of patients in either group were neutropenia (88.9% and 90.7% in the inavolisib and placebo groups, respectively), stomatitis/mucosal inflammation (51.2% and 26.5%), hyperglycemia (58.6% and 8.6%), diarrhea (48.1% and 16.0%), and rash (25.3% and 17.3%) (Table B2). Febrile neutropenia was noted in 2.5% and 0.6%, respectively. The incidence of hyperglycemia by body mass index (BMI) was 65.5% for BMI≥30 kg/m² and 56.8% for BMI<30 kg/m².

TABLE B2
Most Common Adverse Events at Any Grade in Either
Treatment Group (≥20%) (Safety Analysis Set).

| | Inavolisib + | | | | Placebo + | | | |
|---|---|---|---|---|---|---|---|---|
| | palbociclib + | | | | palbociclib + | | | |
| | fulvestrant | | | | fulvestrant | | | |
| | (n = 162) | | | | (n = 162) | | | |
| Adverse Event | All grades | | Grades 3-4 | | All grades | | Grades 3-4 | |
| Neutropenia | 144 | (88.9) | 130 | (80.2) | 147 | (90.7) | 127 | (78.4) |
| Thrombocytopenia | 78 | (48.1) | 23 | (14.2) | 73 | (45.1) | 7 | (4.3) |
| Stomatitis/mucosal | 83 | (51.2) | 9 | (5.6) | 43 | (26.5) | 0 | |
| inflammation | | | | | | | | |
| Anemia | 60 | (37.0) | 10 | (6.2) | 59 | (36.4) | 3 | (1.9) |
| Hyperglycemia | 95 | (58.6) | 9 | (5.6) | 14 | (8.6) | 0 | |
| Diarrhea | 78 | (48.1) | 6 | (3.7) | 26 | (16.0) | 0 | |
| Nausea | 45 | (27.8) | 1 | (0.6) | 27 | (16.7) | 0 | |
| Rash | 41 | (25.3) | 0 | | 28 | (17.3) | 0 | |
| Decreased appetite | 38 | (23.5) | 0 | | 14 | (8.6) | 0 | |
| Fatigue | 38 | (23.5) | 0 | | 21 | (13.0) | 2 | (1.2) |
| COVID-19 | 37 | (22.8) | 3 | (1.9) | 17 | (10.5) | 1 | (0.6) |
| Headache | 34 | (21.0) | 0 | | 22 | (13.6) | 0 | |
| Leukopenia | 28 | (17.3) | 11 | (6.8) | 40 | (24.7) | 17 | (10.5) |

| | | | | | | |
|---|---|---|---|---|---|---|
| Ocular toxicities | 36 | (22.2) | 0 | 21 | (13.0) | 0 |

Grade 3-4 adverse events were reported in 88.3% and 82.1% in the inavolisib and placebo groups respectively. Neutropenia was reported at grades 3-4 in 80.2% and 78.4% of patients, respectively; stomatitis/mucosal inflammation, 5.6% and 0%; hyperglycemia, 5.6% and 0%; and diarrhea, 3.7% and 0%. No grade 3-4 rash was reported.

Proportions of patients experiencing serious adverse events were 24.1% versus 10.5% in the inavolisib and placebo groups, respectively.

Grade 5 (fatal) adverse events were reported in 3.7% of patients and 1.2% in the inavolisib and placebo groups, respectively. In the inavolisib group, these were acute coronary syndrome, COVID-19, cerebral hemorrhage, cerebrovascular accident, gastrointestinal hemorrhage, and death. In the placebo group, these were cardiac arrest and COVID-19 pneumonia. In both groups, these events were single occurrences, and none were considered related to study treatments by the investigator.

Adverse events led to discontinuation of any treatment in 6.8% of patients in the inavolisib group (6.2% for inavolisib; 4.9% for palbociclib; 3.1% for fulvestrant) and 0.6% in the placebo group (placebo only; no patients discontinued palbociclib/fulvestrant due to adverse events). Adverse events leading to inavolisib or placebo dose reductions occurred in 14.2% and 3.1% of patients, respectively. Hyperglycemia (2.5%) was the only adverse event occurring in ≥2% of patients leading to dose reduction of inavolisib.

### DISCUSSION

INAVO120 met its primary end point, demonstrating that the addition of inavolisib to palbociclib and fulvestrant resulted in substantially longer progression-free survival versus placebo, palbociclib, and fulvestrant in patients with PIK3CA-mutated, HR+, HER2− LA/mBC whose disease recurred on or within 12 months of adjuvant endocrine therapy completion. Inavolisib benefit was generally observed across all key prespecified clinical subgroups and sensitivity analyses, and supported by secondary end points, with meaningful improvements in response rate and duration. Overall survival analysis showed a numerical trend in favor of the inavolisib group at the interim analysis, with further follow-up ongoing. Importantly, inavolisib plus palbociclib and fulvestrant had a safety profile consistent with those of the individual drugs,^{11,24-26,30-34} and a low treatment discontinuation rate due to adverse events.

The patient population enrolled in INAVO120 was enriched with poor prognostic factors. INAVO120 enrolled patients with endocrine-resistant and measurable disease, leading to a high proportion of patients with greater disease burden, including involvement of ≥3 organs, visceral disease (80.0%), and liver metastases (51.7%). Additionally, a unique aspect of the INAVO120 study is that >90% of patients' tumor PIK3CA mutation statuses were determined through ctDNA-based testing. These poorer prognostic features of the INAVO120 population are reflected in the performance of the control arm and underscore the improvement due to the addition of inavolisib. The criteria used to define endocrine resistance are internationally established based on consensus.28 Endocrine resistance is a continuum of presentations, which can be attributed to and described by multiple, complex contributing mechanisms. Robust preclinical data and early-phase clinical data suggest that this inavolisib-based combination may be effective in a broader population beyond just endocrine therapy resistant disease.^{17,22-24}

The substantial clinical benefit observed in INAVO120 can be attributed to the simultaneous blockade of the three critical signaling pathways that drive PIK3CA-mutated, HR+, HER2− LA/mBC—the PI3K, ER, and CDK4/6 pathways—thus, delaying and preventing the emergence of treatment resistance driven mainly by cross-talk among these pathways. Moreover, the clinical benefit in the inavolisib group was early and durable, as evidenced by the early separation of Kaplan-Meier curves for progression-free survival at the time of the first tumor assessment which was maintained throughout the follow-up period. In addition, the landmark death rate at 6 months was 10.1% and 2.7% in the placebo and inavolisib groups, respectively, which emphasizes the importance of treating this patient population with this inavolisib-based combination as a first-line therapy.

INAVO120 establishes that inavolisib can be combined with a CDK4/6 inhibitor and endocrine therapy at the full doses of all individual drugs with an acceptable level of safety and tolerability. Hyperglycemia was more frequent in the inavolisib group and with a slightly higher incidence in patients with a BMI≥30 kg/m²; this can be attributed to the fact that hyperglycemia is an on-target toxicity associated with PI3K pathway inhibitors. Other adverse events commonly associated with PI3K inhibitors, namely diarrhea, stomatitis, and rash, were also more frequent in the inavolisib group. However, these key selected adverse events were controlled with supportive care and dose modifications, which is reflected in the high dose intensity for all individual drugs. Moreover, the INAVO120 protocol allowed prophylactic use of metformin in patients at high risk of hyperglycemia and recommended early use of dexamethasone mouthwash as treatment and then prophylaxis for stomatitis, based on the SWISH study.³⁵ Dexamethasone mouthwash prophylaxis from the start of treatment may be appropriate in routine practice, and this will be investigated in future studies. Furthermore, no grade 3-4 rash, no grade 3-4 pneumonitis, and no colitis were reported, whilst neutropenia, including high-grade events, was similar between the inavolisib and placebo groups, with febrile neutropenia being low in both groups.

Although the treatment algorithm of HR+LA/mBC has evolved with approved combinations that target the PI3K/AKT/mTOR pathway, none of these are currently preferred options in the first-line setting for patients with PIK3CA-mutated tumors. Alpelisib, a PI3Kα inhibitor, and everolimus, an mTOR inhibitor, are recommended treatment options in second and later lines, in part because of their challenging safety and tolerability. For example, treatment discontinuation rates due to adverse events were 25.0% and 19% for alpelisib and everolimus combinations, respectively,^{11,13} whereas the treatment discontinuation rate due to adverse events was 6.8% in INAVO120 in the inavolisib group. Cross-trial comparisons should be made with caution due to differences in trial design, patient populations, and analysis and reporting. However, it is notable that attempts to combine alpelisib or everolimus with a CDK4/6 inhibitor and endocrine therapy have been unsuccessful, mainly because of toxicity.^{19,36} Recently, capivasertib, an AKT inhibitor, was approved in combination with fulvestrant for the treatment of patients with HR+, HER2− LA/mBC harboring≥1 PIK3CA/AKT1/PTEN alteration, following progression or recurrence on an endocrine-based regimen.¹² In CAPItello-291, the treatment discontinuation rate due to adverse events with capivasertib plus fulvestrant was 13.0%.¹²

This phase 3 study showed a substantially improved benefit in median progression-free survival and a low rate of treatment discontinuation due to toxicity with a combination of a PI3Kα inhibitor (inavolisib), a CDK4/6 inhibitor, and endocrine therapy (all at full doses) in patients with PIK3CA-mutated, HR+, HER2− LA/mBC. This combination may represent a new treatment option for these patients.

### REFERENCES

1. Anderson E J, Mollon L E, Dean J L, et al. A systematic review of the prevalence and diagnostic workup of PIK3CA mutations in HR+/HER2− metastatic breast cancer. Int J Breast Cancer 2020; 2020:3759179.
2. Miller T W, Balko J M, Arteaga C L. Phosphatidylinositol 3-kinase and antiestrogen resistance in breast cancer. J Clin Oncol 2011; 29:4452-61.
3. LoRusso P M. Inhibition of the PI3K/AKT/mTOR pathway in solid tumors. J Clin Oncol 2016; 34:3803-15.
4. Martinez-Sáez O, Chic N, Pascual T, et al. Frequency and spectrum of PIK3CA somatic mutations in breast cancer. Breast Cancer Res 2020; 22:45.
5. Fillbrunn M, Signorovitch J, André F, et al. PIK3CA mutation status, progression and survival in advanced HR+/HER2− breast cancer: a meta-analysis of published clinical trials. BMC cancer 2022; 22:1002.
6. Hanker A B, Sudhan D R, Arteaga C L. Overcoming endocrine resistance in breast cancer. Cancer Cell 2020; 37:496-513.
7. Zardavas D, Te Marvelde L, Milne R L, et al. Tumor PIK3CA Genotype and Prognosis in Early-Stage Breast Cancer: A Pooled Analysis of Individual Patient Data. J Clin Oncol 2018; 36:981-90.
8. Cristofanilli M, Turner N C, Bondarenko I, et al. Fulvestrant plus palbociclib versus fulvestrant plus placebo for treatment of hormone-receptor-positive, HER2-negative metastatic breast cancer that progressed on previous endocrine therapy (PALOMA-3): Final analysis of the multicentre, double-blind, phase 3 randomised controlled trial. Lancet Oncol 2016; 17:425-39.
9. Slamon D J, Neven P, Chia S K L, et al. Updated overall survival (OS) results from the phase III MONALEESA-3 trial of postmenopausal patients (pts) with HR+/HER2− advanced breast cancer (ABC) treated with fulvestrant (FUL)±ribociclib (RIB). J Clin Oncol 2021; 39:Abstract 1001.
10. Sledge G W, Jr., Toi M, Neven P, et al. MONARCH 2: Abemaciclib in combination with fulvestrant in women with HR+/HER2− advanced breast cancer who had progressed while receiving endocrine therapy. J Clin Oncol 2017; 35:2875-84.
11. André F, Ciruelos E, Rubovszky G, et al. Alpelisib for PIK3CA-mutated, hormone receptor-positive advanced breast cancer. N Engl J Med 2019; 380:1929-40.
12. Turner N C, Oliveira M, Howell S J, et al. Capivasertib in hormone receptor-positive advanced breast cancer. N Engl J Med 2023; 388:2058-70.
13. Baselga J, Campone M, Piccart M, et al. Everolimus in postmenopausal hormone-receptor-positive advanced breast cancer. N Eng J Med 2011; 366:520-9.
14. Presti D, Quaquarini E. The PI3K/AKT/mTOR and CDK4/6 pathways in endocrine resistant HR+/HER2− metastatic breast cancer: biological mechanisms and new treatments. Cancers (Basel) 2019; 11:1242.
15. Herrera-Abreu M T, Palafox M, Asghar U, et al. Early adaptation and acquired resistance to CDK4/6 inhibition in estrogen receptor-positive breast cancer. Cancer Res 2016; 76:2301-13.
16. Vora S R, Juric D, Kim N, et al. CDK 4/6 inhibitors sensitize PIK3CA mutant breast cancer to PI3K inhibitors. Cancer cell 2014; 26:136-49.
17. Song K W, Edgar K A, Hanan E J, et al. RTK-dependent inducible degradation of mutant PI3Kα drives GDC-0077 (inavolisib) efficacy. Cancer Discov 2022; 12:204-19.
18. Juric D, Ismail-Khan R, Campone M, et al. Phase Ib/II study of ribociclib and alpelisib and letrozole in ER+, HER2− breast cancer: safety, preliminary efficacy and molecular analysis. Cancer Res 2016; 76:Abstract P3-14-01.
19. Tolaney S M, Im Y H, Calvo E, et al. Phase Ib study of ribociclib plus fulvestrant and ribociclib plus fulvestrant plus PI3K inhibitor (alpelisib or buparlisib) for HR(+) advanced breast cancer. Clin Cancer Res 2021; 27:418-28.
20. Pascual J, Lim J S J, Macpherson I R, et al. Triplet therapy with palbociclib, taselisib, and fulvestrant in PIK3CA-mutant breast cancer and doublet palbociclib and taselisib in pathway-mutant solid cancers. Cancer Discov 2021; 11:92-107.
21. Dent S, Cortes J, Im Y H, et al. Phase III randomized study of taselisib or placebo with fulvestrant in estrogen receptor-positive, PIK3CA-mutant, HER2-negative, advanced breast cancer: the SANDPIPER trial. Ann Oncol 2021; 32:197-207.
22. Edgar K, Hong R, Song K, et al. GDC-0077 is a selective PI3K alpha inhibitor with robust efficacy in PIK3CA mutant hormone-positive breast cancer models. Cancer Res 2020; 80:Abstract P3-11-23 and associated poster presentation.
23. Hanan E J, Braun M G, Heald R A, et al. Discovery of GDC-0077 (inavolisib), a highly selective inhibitor and degrader of mutant PI3Kα. J Med Chem 2022; 65:16589-621.
24. Bedard P L, Jhaveri K, Cervantes A, et al. A phase I/Ib study evaluating GDC-0077+palbociclib (palbo)+fulvestrant in patients (pts) with PIK3CA-mutant (mut), hormone receptor-positive/HER2-negative metastatic breast cancer (HR+/HER2− mBC). Cancer Res 2021; 81:Abstract PD1-02 and associated poster presentation.
25. Juric D, Kalinsky K, Oliveira M, et al. A first-in-human phase Ia dose escalation study of GDC-0077, a p110a-selective and mutant-degrading PI3K inhibitor, in patients with PIK3CA-mutant solid tumors. Cancer Res 2020; 80:Abstract OT1-08-4 and associated poster presentation.
26. Jhaveri K, Kalinsky K, Bedard P, et al. A phase Ib dose escalation study evaluating the mutant selective PI3K-alpha inhibitor GDC-0077 (G) in combination with letrozole (L) with and without palbociclib (P) in patients with PIK3CA-mutant HR+/HER2− breast cancer. Cancer Res 2020; 80:Abstract P1-19-46.
27. Eisenhauer E A, Therasse P, Bogaerts J, et al. New response evaluation criteria in solid tumours: revised RECIST guideline (version 1.1). Eur J Cancer 2009; 45:228-47.
28. Cardoso F, Senkus E, Costa A, et al. 4th ESO-ESMO International Consensus Guidelines for Advanced Breast Cancer (ABC 4)†. Ann Oncol 2018; 29:1634-57.
29. Common Terminology Criteria for Adverse Events (CTCAE) Version 5.0. National Cancer Institute (NCI): Division of Cancer Treatment & Diagnosis (DCTD), (https://ctep.cancer.gov/protocoldevelopment/electronic_applications/docs/CTCAE_v5_Quick_Reference_5x7.pdf.)
30. Oliveira M, Jhaveri K, Juric D, et al. Targeted safety events from a phase I/Ib study evaluating GDC-0077 alone and in combination with endocrine therapy (ET)±palbociclib (palbo) in patients (pts) with PIK3CA-mutant (mut), hormone receptor-positive/HER2− negative metastatic breast cancer (HR+/HER2− mBC). Cancer Res 2021; 81:Abstract PSi11- and associated poster presentation.
31. Bedard P L, Accordino M K, Cervantes A, et al. Long-term safety of inavolisib (GDC-0077) in an ongoing phase 1/1b study evaluating monotherapy and in combination (combo) with palbociclib (palbo) and/or endocrine therapy in patients (pts) with PIK3CA-mutated, hormone receptor-positive/HER2-negative (HR+/HER2−) metastatic breast cancer (BC). J Clin Oncol 3-7 Jun. 2022. Abstract 1052; 40:Abstract 1052.
32. Kalinsky K, Juric D, Bedard P L, et al. A phase I/Ib study evaluating GDC-0077 plus fulvestrant in patients with PIK3C A-mutant, hormone receptor-positive/HER2-negative breast cancer. Cancer Res 2020; 80:Abstract CT109 and associated oral presentation.
33. Juric D, Bedard P L, Cervantes A, et al. A phase I/Ib study of inavolisib (GDC-0077) in combination with fulvestrant in patients (pts) with PIK3CA-mutated hormone receptor-positive/HER2-negative (HR+/HER2−) metastatic breast cancer. Cancer Res 2022; 82:Abstract P5-17-05.
34. Di Leo A, Jerusalem G, Petruzelka L, et al. Final overall survival: fulvestrant 500 mg vs 250 mg in the randomized CONFIRM trial. J Natl Cancer Inst 2014; 106:djt337.
35. Rugo H S, Seneviratne L, Beck J T, et al. Prevention of everolimus-related stomatitis in women with hormone receptor-positive, HER2-negative metastatic breast cancer using dexamethasone mouthwash (SWISH): a single-arm, phase 2 trial. Lancet Oncol 2017; 18:654-62.
36. Bardia A, Hurvitz S A, DeMichele A, et al. Phase I/II trial of exemestane, ribociclib, and everolimus in women with HR⁺/HER2⁻ advanced breast cancer after progression on CDK4/6 inhibitors (TRINITI-1). Clin Cancer Res 2021; 27:4177-85.

### Additional Embodiments

In general, any aspects or embodiments that are described herein in terms of methods of treatment may also be defined in terms of the compounds or combinations for use in the treatment of the specified condition and in relation to any specified patient characteristics, e.g. a specified patient population. Such compounds or combinations for use in the defined treatment in relation to any specified patient characteristics also form part of the present invention.

Specific aspects of the present invention are defined in the following numbered statements.

1A. A method of treating hormone receptor positive and HER2 negative locally advanced or metastatic breast cancer in a patient having PIK3CA mutated, hormone receptor positive and HER2 negative locally advanced or metastatic breast cancer, the method comprising administering to the patient a combination therapy comprising:
(i) inavolisib;
(ii) palbociclib; and
(iii) fulvestrant,
   wherein the combination therapy produces a statistically significant and clinically meaningful improvement to the patient compared to administering palbociclib and fulvestrant alone to a comparable patient.

2A. A method of treating hormone receptor positive and HER2 negative locally advanced or metastatic breast cancer in a patient having PIK3CA mutant, hormone receptor positive and HER2 negative locally advanced or metastatic breast cancer, the method comprising administering to the patient a combination therapy comprising a dosing regimen comprising:
a. administering inavolisib QD on days 1-28 of a first 28-day cycle;
b. administering palbociclib QD on days 1-21 of a first 28-day cycle; and
c. administering fulvestrant on days 1 and 15 of a first 28-day cycle,
   wherein the combination therapy produces a statistically significant and clinically meaningful improvement to the patient compared to administering palbociclib and fulvestrant alone to a comparable patient.

3A. The method of 1A or 2A, further comprising one or more additional 28-day cycles comprising:
a. administering inavolisib on days 1-28 of each additional 28-day cycle;
b. administering palbociclib on days 1-21 of each additional 28-day cycle; and
c. administering fulvestrant on day 1 of each additional 28-day cycle.

4A. The method of any one of 1A-3A, wherein inavolisib is administered at an amount of 9 mg.

5A. The method of 4A, wherein inavolisib is administered at an amount of 9 mg in an oral tablet.

6A. The method of any one of 1A-5A, wherein palbociclib is administered at an amount of 125 mg in an oral capsule or tablet.

7A. The method of any one of 1A-6A, wherein fulvestrant is administered at an amount of 500 mg by intramuscular (IM) infusion.

8A. A method of inhibiting tumor growth or producing/increasing tumor regression in a patient having PIK3CA mutant, hormone receptor positive and HER2 negative locally advanced or metastatic breast cancer, the method comprising administering to the patient a combination therapy according to the methods of any one of 1A-7A.

9A. The method of any one of 1A-8A, wherein the patient has locally advanced or metastatic breast cancer not amenable to curative therapy.

10A. The method of any one of 1A-9A, wherein the breast cancer is endocrine resistant.

11A. The method of any one of 1A-10A, wherein the patient has progression of disease during adjuvant endocrine treatment or within 12 months of completing adjuvant endocrine therapy.

12A. The method of 11A, wherein the patient has progression of disease during the first two years of adjuvant endocrine treatment.

13A. The method of 11A or 12A, wherein the adjuvant endocrine therapy is with an aromatase inhibitor or tamoxifen.

14A. The method of 13A, wherein the adjuvant endocrine therapy is tamoxifen only.

15A. The method of any one of 1A-14A, wherein the patient has adequate hematologic and organ function within 14 days prior to initiation of treatment.

16A. The method of any one of 1A-15A, wherein the patient is not post-menopausal.

17A. The method of any one of 1A-16A, wherein the patient has PIK3CA mutated, hormone receptor positive and HER2 negative metastatic breast cancer.

18A. The method of any one of 1A-17A, wherein the patient has PIK3CA mutated, estrogen receptor positive, progesterone receptor positive, and HER2 negative locally advanced or metastatic breast cancer.

19A. The method of any one of 1A-18A, wherein the patient is an adult.

20A. The method of any one of 1A-19A, wherein the patient is younger than 65 years of age.

21A. The method of any one of 1A-20A, wherein the breast cancer is as detected by an FDA-approved test.

22A. A method of preventing or delaying development of resistance of a breast cancer to a therapy containing palbociclib, said method comprising administering a combination therapy comprising inavolisib, palbociclib and fulvestrant, according to the method of any one of 1A-21A.

23A. A method of treating an endocrine-resistant, PIK3CA mutated, hormone receptor (HR)-positive, human epidermal growth factor receptor 2 (HER2)-negative, locally advanced or metastatic breast cancer in an adult patient, the method comprising administering to the patient a combination therapy comprising:
(i) inavolisib;
(ii) palbociclib; and
(iii) fulvestrant,
   following recurrence on or after completing an adjuvant endocrine therapy.

24A. The method of 23A, wherein the breast cancer is as detected by an FDA-approved test.

Many modifications and other embodiments of the inventions set forth herein will come to mind to one skilled in the art to which these inventions pertain having the benefit of the teachings presented in the foregoing descriptions and the associated drawings. Therefore, it is to be understood that the inventions are not to be limited to the specific embodiments disclosed and that modifications and other embodiments are intended to be included within the scope of the appended claims. Although specific terms are employed herein, they are used in a generic and descriptive sense only and not for purposes of limitation.

We claim:

1. A method of treating hormone receptor positive and HER2 negative locally advanced or metastatic breast cancer in a patient having PIK3CA mutated, hormone receptor positive and HER2 negative locally advanced or metastatic breast cancer, the method comprising administering to the patient a combination therapy comprising:
(i) inavolisib;
(ii) palbociclib; and
(iii) fulvestrant,
wherein the combination therapy produces a statistically significant and clinically meaningful improvement to the patient compared to administering palbociclib and fulvestrant alone to a comparable patient.

2. The method of claim 1, comprising administering to the patient a combination therapy comprising a dosing regimen comprising:
a. administering inavolisib QD on days 1-28 of a first 28-day cycle;
b. administering palbociclib QD on days 1-21 of a first 28-day cycle; and
c. administering fulvestrant on days 1 and 15 of a first 28-day cycle.

3. The method of claim 2, further comprising one or more additional 28-day cycles comprising:
a. administering inavolisib on days 1-28 of each additional 28-day cycle;
b. administering palbociclib on days 1-21 of each additional 28-day cycle; and
c. administering fulvestrant on day 1 of each additional 28-day cycle.

4. The method of claim 1, wherein inavolisib is administered at an amount of 9 mg in an oral tablet, wherein palbociclib is administered at an amount of 125 mg in an oral capsule or tablet, or wherein palbociclib is administered at an amount of 125 mg in an oral capsule or tablet.

5. A method of inhibiting tumor growth or producing/increasing tumor regression in a patient having PIK3CA mutant, hormone receptor positive and HER2 negative locally advanced or metastatic breast cancer, the method comprising administering to the patient a combination therapy according to the method of claim 1.

6. The method of claim 1, wherein the patient has locally advanced or metastatic breast cancer not amenable to curative therapy.

7. The method of claim 1, wherein the breast cancer is endocrine resistant.

8. The method of claim 1, wherein the patient has progression of disease during adjuvant endocrine treatment or within 12 months of completing adjuvant endocrine therapy.

9. The method of claim 8, wherein the patient has progression of disease during the first two years of adjuvant endocrine treatment.

10. The method of claim 8, wherein the adjuvant endocrine therapy is with an aromatase inhibitor or tamoxifen.

11. The method of claim 10, wherein the adjuvant endocrine therapy is tamoxifen only.

12. The method of claim 1, wherein the patient has adequate hematologic and organ function within 14 days prior to initiation of treatment.

13. The method of claim 1, wherein the patient is not post-menopausal.

14. The method of claim 1, wherein the patient is an adult.

15. The method of claim 1, wherein the breast cancer is as detected by an FDA-approved test.

16. A method of preventing or delaying development of resistance of a breast cancer to a therapy containing palbociclib, said method comprising administering a combination therapy comprising inavolisib, palbociclib and fulvestrant, according to the method of claim 1.

17. A method of treating an endocrine-resistant, PIK3CA mutated, hormone receptor (HR)-positive, human epidermal growth factor receptor 2 (HER2)-negative, locally advanced or metastatic breast cancer in an adult patient, the method comprising administering to the patient a combination therapy comprising inavolisib, palbociclib and fulvestrant, following recurrence on or after completing an adjuvant endocrine therapy.

18. The method of claim 17, wherein the breast cancer is as detected by an FDA-approved test.

19. The method of claim 17, comprising administering to the patient a combination therapy comprising a dosing regimen comprising:
a. administering inavolisib QD on days 1-28 of a 28-day cycle;
b. administering palbociclib QD on days 1-21 of a 28-day cycle; and
c. administering fulvestrant on days 1 and 15 of a 28-day cycle.

20. The method of claim 17, wherein the adjuvant endocrine therapy is with an aromatase inhibitor or tamoxifen, wherein the patient is not post-menopausal, or wherein the patient is younger than 65 years of age.
